(19) United States
(12) Patent Application Publication
(10) Pub. No.: US 2025/0127722 A1
(43) Pub. Date: Apr. 24, 2025

(54) SOLID ORAL DOSAGE FORM COMPRISING ANTIBODIES FOR SUSTAINED RELEASE IN THE LOWER GASTROINTESTINAL TRACT

(71) Applicant: Tillotts Pharma AG, Rheinfelden (CH)

(72) Inventors: Roberto Carlos BRAVO GONZALEZ, Rheinfelden (CH); Ramon VOGLER, Rheinfelden (CH)

(73) Assignee: Tillotts Pharma AG, Rheinfelden (CH)

(21) Appl. No.: 18/904,399

(22) Filed: Oct. 2, 2024

Related U.S. Application Data
(63) Continuation-in-part of application No. PCT/EP2024/058606, filed on Mar. 28, 2024.
(60) Provisional application No. 63/492,513, filed on Mar. 28, 2023.

(51) Int. Cl. A61K 9/16; A61K 47/26; C07K 16/24

(52) U.S. Cl. CPC A61K 9/1676; A61K 9/1611; A61K 9/1623; A61K 9/1635; A61K 9/1652; A61K 47/26; C07K 16/241

(57) ABSTRACT

The present invention relates to a solid oral dosage form for sustained release in the lower gastrointestinal tract, comprising antibodies or functional fragments thereof in a depot layer (2) covering an inert core unit (1), a sustained release layer (3) covering the depot layer, and a delayed release layer (4) covering the sustained release layer, preferably prepared by drug layering; an oral multiparticulate drug delivery system comprising a plurality of the solid oral dosage forms; and the use of the solid oral dosage form in the targeted local treatment in the lower gastrointestinal tract of a patient.

### PRIORITY

The instant application is continuation-in-part of PCT/EP2024/058606 filed Mar. 28, 2024, which, in turn, claims the benefit of U.S. Provisional Application No. 63/492,513 filed Mar. 28, 2023. The contents of both prior applications are incorporated by reference herein in their entirety.

### SEQUENCE LISTING

The instant application contains a Sequence Listing compliant with WIPO Standard ST.26. The Sequence Listing, entitled “LNK_245CIP_SL.xml”, was created Oct. 2, 2024, is 52025 bytes in size and is hereby incorporated by reference in its entirety.

### FIELD OF THE INVENTION

The present invention relates to a solid oral dosage form for sustained release in the lower gastrointestinal tract, comprising antibodies or functional fragments thereof specific to tumor necrosis factor alpha (TNFα) in a depot layer (2) covering an inert core unit (1), a sustained release layer (3) covering the depot layer, and a delayed release layer (4) covering the sustained release layer, preferably prepared by drug layering; an oral multiparticulate drug delivery system comprising a plurality of the solid oral dosage forms; and the use of the solid oral dosage form in the targeted local treatment in the lower gastrointestinal tract of a patient.

### BACKGROUND OF THE INVENTION

Solid dosage forms such as pellets, mini-tablets, tablets, granules, capsules or tablets and the like are widely used in pharmaceutical industry. They typically comprise at least one active ingredient, and one or more carriers and other excipients. Advantages of such solid dosage forms include less storage space, ease of handling, and improved stability. Moreover, tablets or capsules provide the most widely used dosage unit for applying drugs to a patient in a non-invasive manner. For small molecule drugs there exists a long-established practice of preparing solid dosage forms. For antibodies and functional fragments thereof on the other hand, although they are finding an ever-increasing use as active ingredients in therapeutic or diagnostic applications, the formulation into solid dosage forms is more challenging.

As an alternative to systemic treatment, the use of solid dosage forms intended for oral administration and targeting parts of the gastrointestinal (GI) tract has many potential advantages and therefore has become an interesting alternative in recent years, since it allows for the targeted local treatment of symptoms of diseases in parts of the GI tract, as for example immune checkpoint inhibitor (ICPI) induced colitis, ICPI induced enterocolitis, ICPI induced diarrhoea, inflammatory bowel disease (IBD), colorectal cancer, diarrhea or microbial infections. However, the use of antibodies and functional fragments of antibodies in solid dosage forms for such targeted local treatment in the GI tract faces various challenges.

Colitis is as a disorder characterized by inflammation of the large intestine/colon. Enteritis is defined as a disorder characterized by inflammation of the small intestine. Enterocolitis is as a disorder characterized by inflammation of both the small intestine and the large intestine/colon, i.e. a combination of enteritis and colitis. Symptoms characterizing ICP inhibitor-induced colitis include diarrhoea, abdominal pain, nausea, cramping, blood or mucus in stool or changes in bowel habits, fever, abdominal distention, obstipation and constipation (Brahmer et al., Management of Immune-Related Adverse Events in Patients Treated With Immune Checkpoint Inhibitor Therapy: American Society of Clinical Oncology (ASCO) Practice Guideline, J Clin Oncol. (2018) 10; 36(17):1714-1768). Symptoms characterizing ICP inhibitor-induced enterocolitis include the symptoms listed above for ICP inhibitor-induced colitis.

With respect to ICPI induced colitis, ICPI induced enterocolitis and ICPI induced diarrhoea, it is important that patients can be treated for ICPI induced colitis, ICPI induced enterocolitis or ICPI induced diarrhoea with an antibody or functional antibody fragment while still undergoing cancer treatment with ICPI via intravenous route. This requires an alternative route of administration, both to allow parallel intravenous cancer treatment (for example, the standard therapy for treatment of severe ICPI induced colitis or ICPI induced enterocolitis using anti-TNFα specific antibodies at present involves regular systemic administration of an anti-TNFα antibody by intravenous infusion, which requires interruption of ICPI treatment), and to ensure targeted local delivery to keep systemic exposure to the antibody and potential side effects resulting therefrom to an absolute minimum. While this is beneficial for all patients, it is particularly important for patients with ICPI-induced colitis, ICPI induced enterocolitis or ICPI induced diarrhoea who are already heavily burdened by cancer therapy.

A problem associated with solid oral dosage forms comprising antibodies and functional fragments of antibodies is the size of the antibodies and antibody fragments compared to small drug molecules, which, when combined with functional coatings like sustained release coatings and/or delayed release coatings, can greatly affect the release behaviour.

The targeted release of therapeutic antibodies or functional fragments thereof from a solid dosage form in a specific part of the GI tract over a defined window of time is particularly desirable for diseases of the lower GI tract and in particular ICPI induced colitis, ICPI induced enterocolitis, ICPI induced diarrhoea and IBD. However, a targeted release of antibodies or functional fragments thereof from a solid dosage form in a specific part of the GI tract over a defined window of time can also be very challenging.

Thus, there is a need for a solid oral dosage form comprising therapeutic antibodies, preferably specific to tumor necrosis factor alpha (TNFα), or functional fragments thereof, for release in a defined part of the GI tract and in particular the lower GI tract, that ensures targeted release over a defined window of time.

### SUMMARY OF THE INVENTION

After testing various solid dosage form designs, excipients, manufacturing methods and processing conditions the present inventors found an advantageous solid oral dosage form comprising an antibody or a functional fragment thereof, preferably specific to tumor necrosis factor alpha (TNFα), as an active ingredient. The solid oral dosage form of the present invention comprises an inert core unit (1), a depot layer (2) comprising the antibody or functional fragment thereof, a sustained release layer (3), and a delayed release layer (4). The ingredients and composition of the depot layer, sustained release layer and delayed release layer are carefully selected to ensure the desired release profile of the antibody or functional fragment thereof after oral administration.

The present inventors unexpectedly found that a favorable release profile can be obtained when the delayed release layer comprises an anionic polymer and the sustained release layer comprises a cationic polymer. A higher amount of cationic groups in the polymer of the sustained release layer was found to lead to a higher amount of antibody released within 24 hours.

Thus, the final solid oral dosage form found by the present inventors works due to the combinatorial effect of the layers and of the carefully selected and calibrated components in the different layers, enabling the targeted release of the antibody or functional fragment thereof in a desired manner.

In addition, the formulation of the present invention was shown to be stable for 12 months. Unexpectedly, the antibody activity was stable even after storage at 25° C./60% RH for one year.

The solid oral dosage form of the present invention prevents release of the antibody or functional fragment thereof before a target site in the gastrointestinal (GI) tract of a patient. Thereby, the solid oral dosage form advantageously ensures a controlled and sustained release from the solid oral dosage form in the lower part of the GI tract, particularly starting preferably in the terminal ileum or the ileocolonic region, and over a clearly defined period of time, e.g. over the course of a day, ensuring the release of the majority of the antibody or functional fragment thereof from the solid oral dosage form in the target region of the GI tract and before it is expelled via the anus. Thus, the solid oral dosage form ensures that an optimal amount of the antibody or functional fragment thereof in active form can be released from the solid oral dosage form in a controlled manner. The solid oral dosage form is preferably prepared by drug layering, using carefully chosen combinations of excipients that ensure a fast and straight forward preparation of the solid oral dosage form, while preserving stability and activity of the antibodies or functional fragments thereof.

Thus, the present invention provides a novel solid oral dosage form, comprising as an active agent an antibody or functional fragment thereof, preferably specific to TNFα, in a depot layer (2) covering an inert core unit (1), a sustained release layer (3) covering the depot layer and a delayed release layer (4) covering the sustained release layer, thereby ensuring carefully calibrated delayed and sustained release from the solid oral dosage form starting at a defined location in the lower GI tract. The present invention relates to the subject matter defined in the following items 1 to 131:

The present invention relates to the subject matter defined in the following items 1 to 131:

[1] A solid oral dosage form comprising
i) an inert core unit (1);
ii) a depot layer (2) covering the inert core unit (1) and comprising an antibody or a functional fragment thereof, as an active agent; and optionally a stabilizer, a buffer and/or a polymeric binder;
iii) a sustained release layer (3), covering the depot layer (2) and comprising at least one cationic polymer; and optionally a plasticizer and/or an anti-tacking agent; and
iv) a delayed release layer (4) covering the sustained release layer (3) and comprising at least one anionic polymer and optionally a plasticizer.

[2] A solid oral dosage form comprising
i) an inert core unit (1);
ii) a depot layer (2) covering the inert core unit (1) and comprising an antibody or a functional fragment thereof as an active agent; a stabilizer; a buffer; and a polymeric binder;
iii) a sustained release layer (3), covering the depot layer (2) and comprising at least one cationic polymer; a plasticizer; and an anti-tacking agent; and
iv) a delayed release layer (4) covering the sustained release layer (3) and comprising at least one anionic polymer and a plasticizer.

[3] The solid dosage form of item 1 or 2, wherein the at least one anionic polymer is selected from the group consisting of polymers comprising carboxylic acid groups; poly(methacrylic acid, methyl methacrylate) 1:1; poly(methacrylic acid, ethyl acrylate) 1:1; poly(methacrylic acid, methyl methacrylate) 1:2; poly(methyl acrylate, methyl methacrylate, methacrylic acid) 7:3:1; carboxymethyl cellulose; and combinations thereof.

[4] The solid dosage form of any one of items 1 to 3, wherein the at least one cationic polymer is selected from the group consisting of chitosan; cellulose; ammonio methacrylate copolymers; poly(ethyl acrylate, methyl methacrylate, trimethylammonioethyl methacrylate chloride) 1:2:0.1; poly(2-N,N-dimethylaminoethylmethacrylate); poly-L-lysine; polyethylenimine; Poly(amidoamine).

[5] A solid oral dosage form comprising
i) an inert core unit (1);
ii) a depot layer (2) covering the inert core unit (1) and comprising an antibody or a functional fragment thereof, preferably specific to tumor necrosis factor alpha (TNFα), as an active agent; a stabilizer, preferably sucrose; a buffer; and a polymeric binder, preferably hypromellose (HPMC);
iii) a sustained release layer (3), covering the depot layer (2) and comprising an ammonio methacrylate copolymer, preferably poly(ethyl acrylate, methyl methacrylate, trimethylammonioethyl methacrylate chloride) 1:2:0.1; a plasticizer; and an anti-tacking agent; and
iv) a delayed release layer (4) covering the sustained release layer (3) and comprising poly(methacrylic acid, methyl methacrylate) 1:2, poly(methyl acrylate, methyl methacrylate, methacrylic acid) 7:3:1, poly(methacrylic acid, methyl methacrylate) 1:1, or a combination thereof, preferably poly(methacrylic acid, methyl methacrylate) 1:2; an anti-tacking agent, preferably glycerol monostearate (GMS); and a plasticizer, preferably triethyl citrate (TEC).

[6] The solid oral dosage form according to any one of the preceding items, wherein the solid oral dosage form is a pellet, granule, bead, sphere, mini-sphere, tablet or mini-tablet, preferably a pellet.

[7] The solid oral dosage form of any one of the preceding items, which is gastric resistant.

[8] The solid oral dosage form according to any one of the preceding items, for targeted release of the antibody or functional fragment thereof starting in the terminal ileum, the ileocolonic region, the ascending colon or transverse colon, preferably in the terminal ileum, the ileocolonic region or the ascending colon, more preferably in the terminal ileum or the ileocolonic region.

[9] The solid oral dosage form according to any one of the preceding items, for targeted release of the antibody or functional fragment thereof in the terminal ileum, the ileocolonic region, the ascending colon, the transverse colon, and/or the sigmoid colon.

[10] The solid oral dosage form according to any one of the preceding items, for targeted sustained release of the antibody or functional fragment thereof starting in the terminal ileum and continuing at least through the sigmoid colon.

[11] The solid oral dosage form according to any of the preceding items, wherein the inert core unit (1) is an inert pellet, mini-tablet, tablet, granule, core, bead, mini-sphere or sphere, preferably a pellet.

[12] The solid oral dosage form according to any of the preceding items, wherein the inert core unit (1) comprises a monosaccharide, disaccharide, oligosaccharide, polysaccharide, silica, tartaric acid, calcium carbonate, or a combination thereof as a main component.

[13] The solid oral dosage form according to any of the preceding items, wherein the inert core unit (1) consists of a monosaccharide, disaccharide, oligosaccharide, polysaccharide, silica, tartaric acid, calcium carbonate, or a combination thereof.

[14] The solid oral dosage form according to any of the preceding items, wherein the inert core unit (1) is a pellet, preferably comprising, or consisting of, a sphere; consists of a water insoluble or water swellable material; and/or is of uniform composition.

[15] The solid oral dosage form according to any of items 1 to 13, wherein the inert core unit (1) comprises, or consists of, a pellet, e.g. a sphere, and optionally a seal coating covering the sphere, the seal coating preferably consisting of a water-insoluble material.

[16] The solid oral dosage form according to any of the preceding items, wherein the inert core unit (1) comprises a monosaccharide, disaccharide, oligosaccharide, polysaccharide, silica, tartaric acid or a combination thereof as main or only component(s).

[17] The solid oral dosage form according to any of the preceding items, wherein the inert core unit (1), preferably is a pellet and, comprises, or consists of, microcrystalline cellulose, sucrose, starch, mannitol, calcium carbonate, silica, tartaric acid, lactose, carboxymethylcellulose, crosslinked sodium carboxymethylcellulose, or a combination thereof.

[18] The solid oral dosage form according to any of the preceding items, wherein the inert core unit (1) comprises, or consists of, microcrystalline cellulose.

[19] The solid oral dosage form according to any of the preceding items, wherein the inert core unit (1) is pharmaceutically inactive.

[20] The solid oral dosage form according to any of the preceding items, wherein the inert core unit (1) comprises no active agents.

[21] The solid oral dosage form according to any of the preceding items, wherein the inert core unit (1) is a pellet with a sphericity degree of at least 0.6, preferably at least 0.7, more preferably at least 0.8, even more preferably at least 0.9, even more preferably at least 0.95.

[22] The solid oral dosage form according to any of the preceding items, wherein the inert core unit (1) is a pellet with a median particle size of 50-5000 μm, preferably 100-3000 μm, more preferably 200-2000 μm, even more preferably 300-1500 μm, even more preferably 400-1400, even more preferably 700-1400, even more preferably 500-1000, most preferably 500-700, 600-800, or 700-1000 μm.

[23] The solid oral dosage form according to any of the preceding items, wherein the inert core unit (1) is a pellet with a particle size distribution such that at least 85% of the pellets have a particle size of 50-3000 μm, preferably 100-1500 μm, more preferably 350-1400 μm, even more preferably 500-1400 μm, even more preferably 700-1400, even more preferably 500-1000 μm, e.g. 500-700 μm or 700-1000 μm.

[24] The solid oral dosage form according to any of the preceding items, wherein the inert core unit (1) is a pellet with a particle size distribution such that at least 85% of the pellets have a particle size of 700-1400 μm, preferably of 700-1000 μm.

[25] The solid oral dosage form according to any of the preceding items, wherein the depot layer (2) was deposited on the inert core unit (1) by drug layering, preferably using spray coating, more preferably fluidized-bed spray coating.

[26] The solid oral dosage form according to any of the preceding items, wherein the stabilizer in the depot layer (2) is selected from sucrose, maltose, lactose, trehalose, glycerol, maltitol, isomalt, mannitol, sorbitol, xylitol and combinations thereof, preferably sucrose, maltose, lactose, glycerol, maltitol, isomalt, and combinations thereof, more preferably sucrose.

[27] The solid oral dosage form according to any of the preceding items, wherein the buffer (i.e. buffer salt or salts and/or free base) in the depot layer (2) is selected from the group consisting of L-histidine buffer, citrate buffer, hydroxymethylaminomethane (TRIS) buffer, succinate buffer, phosphate buffer, acetate buffer, or salts thereof, and combinations thereof; preferably L-histidine buffer, citrate buffer, TRIS buffer, and combinations thereof; more preferably L-histidine buffer, citrate-TRIS buffer, and a combination thereof.

[28] The solid oral dosage form according to any of the above items, wherein the buffer (i.e. buffer salt and/or free base) in the depot layer (2) comprises, or consists of, L-histidine, preferably L-histidine monohydrochloride and/or free base.

[29] The solid oral dosage form according to any of the preceding items, wherein the polymeric binder in the depot layer (2) is selected from hypromellose (HPMC); methylcellulose (MC); polyvinylpirrolidone (PVP); polyvinylalcohol (PVA); hydroxypropyl cellulose (HPC); macrogol poly(vinylalcohol) grafted copolymer (e.g. Kollidon® IR); and combinations thereof; preferably HPMC or MC; more preferably HPMC.

[30] The solid oral dosage form according to any of the preceding items, wherein the polymeric binder in the depot layer (2) is suitable for an immediate release drug coating.

[31] The solid oral dosage form according to any of the preceding items, wherein the polymeric binder in the depot layer (2) is hypromellose (HPMC); preferably hypromellose 2910, more preferably hypromellose 2910 2.6-3.6 mPas, even more preferably hypromellose 2910 3 mPas.

[32] The solid oral dosage form according to any of the preceding items, wherein the depot layer (2) comprises an anti-tacking agent.

[33] The solid oral dosage form according to item 32, wherein the anti-tacking agent is selected from mesoporous silica, colloidal silica dioxide, stearic acid, magnesium stearate, glycerol monostearate (GMS) and talc, preferably mesoporous silica.

[34] The solid oral dosage form according to any of the preceding items, wherein the depot layer (2) comprises a coalescence enhancer.

[35] The solid oral dosage form according to any of the preceding items, wherein the depot layer (2) comprises a surfactant, preferably a nonionic surfactant.

[36] The solid oral dosage form according to item 35, wherein the surfactant is selected from the group consisting of polysorbate 20, polysorbate 28, polysorbate 40, polysorbate 60, polysorbate 65, polysorbate 80, polysorbate 81, polysorbate 85, poloxamer 124, poloxamer 181, poloxamer 188, poloxamer 237, poloxamer 331, poloxamer 338 and poloxamer 407, and combinations thereof.

[37] The solid oral dosage form according to item 35, wherein the surfactant is polysorbate 20, 28, 40, 60, 65, 80, 81 and 85; preferably polysorbate 80.

[38] The solid oral dosage form according to any of the preceding items, wherein the depot layer (2) comprises or consists of antibody or functional fragment thereof preferably specific to TNFα as an active agent, sucrose, L-histidine (salt and/or free base), preferably L-histidine monohydrochloride and/or free base, hypromellose (HPMC), preferably hypromellose 2910 2.6-3.6 mPas, mesoporous silica, polysorbate 80, and residual water.

[39] The solid oral dosage form according to any of the preceding items, wherein the solid oral dosage form comprises 0.05-30 wt.-%, preferably 0.1-25 wt.-%, more preferably 0.1-20 wt.-%, even more preferably, 0.2-20 wt.-%, even more preferably, 0.2-15 wt.-%, even more preferably 0.5-12 wt.-%, even more preferably 0.5-10 wt.-%, even more preferably 0.8-12 wt.-%, even more preferably 8-12 wt.-%, even more preferably 8-10 wt.-%, e.g. about 9.5 wt.-%, of the antibody or functional fragment thereof preferably specific to TNFα.

[40] The solid oral dosage form according to any of the preceding items, wherein the depot layer (2) comprises 0.2-75 wt.-% antibody or functional fragment thereof, 0.5-65 wt.-% binder, 2-80 wt.-% sucrose, 0.1-10 wt.-% L-histidine (salt and/or free base) and optionally 0.05-10 wt.-% other buffers, 0.2-15 wt.-% anti-tacking agent, 0.01-1 wt.-% surfactant, and/or up to 10 wt.-% water, relative to the total weight of the depot layer; preferably 1-45 wt.-% antibody or functional fragment thereof, 2-35 wt.-% binder, 10-70 wt.-% sucrose, 0.2-5 wt.-% L-histidine and optionally 0.1-4 wt.-% other buffers, 1-5 wt.-% anti-tacking agent, 0.05-0.5 wt.-% surfactant, and/or up to 7 wt.-% water, relative to the total weight of the depot layer; more preferably 10-35 wt.-% antibody or functional fragment thereof, 10-30 wt.-% binder, 30-60 wt.-% sucrose, 0.3-4 wt.-% L-histidine and optionally 0.2-3 wt.-% other buffers, 1.5-3 wt.-% anti-tacking agent, 0.1-0.3 wt.-% surfactant, and/or up to 6 wt.-% water, relative to the total weight of the depot layer; even more preferably 25-35 wt.-% antibody or functional fragment thereof, 15-25 wt.-% binder, 40-55 wt.-% sucrose, 0.4-3 wt.-% L-histidine and optionally 0.5-2.5 wt.-% other buffers, 1.5-2.5 wt.-% anti-tacking agent, 0.2-0.3 wt.-% surfactant, and/or up to 5 wt.-% water, relative to the total weight of the depot layer.

[41] The solid oral dosage form according to any of the preceding items, wherein the solid oral dosage form, comprises, or consists of,
a) in the inert core unit (1), microcrystalline cellulose; and/or
b) in the depot layer (2), antibody or functional fragment thereof preferably specific to TNFα, sucrose, L-histidine, preferably L-histidine monohydrochloride and/or free base, hypromellose (HPMC), preferably hypromellose 2910 2.6-3.6 mPas, mesoporous silica, and/or polysorbate 80; and/or
c) in the sustained release layer (3), poly(ethyl acrylate, methyl methacrylate, trimethylammonioethyl methacrylate chloride) 1:2:0.1 (e.g. Eudragit® RS 30D), triethyl citrate (TEC) and/or mesoporous silica; and/or
d) in the delayed release layer (4), poly(methacrylic acid, methyl methacrylate) 1:2, TEC, polysorbate 80, and/or glycerol monostearate (GMS); and/or
e) residual water in the inert core unit and/or any of the layers.

[42] The solid oral dosage form of any of the preceding items, wherein the inert core unit (1) is a pellet with a particle size distribution such that at least 85% of the pellets have a particle size of 700-1400 μm, preferably 700-1000 μm.

[43] The solid oral dosage form of any of the preceding items, wherein the inert core unit comprises 5-75 wt.-%, preferably 15-65 wt.-%, more preferably 15-60 wt.-%, even more preferably 20-60 wt.-%, even more preferably 25-30 wt.-%, microcrystalline cellulose, relative to the total weight of the solid oral dosage form.

[44] The solid oral dosage form of any of the preceding items, wherein the depot layer (2) comprises, relative to the total weight of the solid oral dosage form, 0.05-30 wt.-% antibody or functional fragment thereof, 0.03-30 wt.-% binder, 0.1-40 wt.-% sucrose, 0.005-5 wt.-% L-histidine (salt and/or free base) and optionally 0.001-5 wt.-% other buffers, 0.005-8 wt.-% anti-tacking agent, and/or 0.0005-2 wt.-% surfactant; preferably 0.1-20 wt.-% antibody or functional fragment thereof, 0.1-25 wt.-% binder, 0.5-35 wt.-% sucrose, 0.01-3 wt.-% L-histidine and optionally 0.01-3 wt.-% other buffers, 0.01-5 wt.-% anti-tacking agent, and/or 0.001-1 wt.-% surfactant; more preferably 0.5-20 wt.-% antibody or functional fragment thereof, 0.5-15 wt.-% binder, 1-25 wt.-% sucrose, 0.05-2 wt.-% L-histidine and optionally 0.05-1.5 wt.-% other buffers, 0.05-1.5 wt.-% anti-tacking agent, and/or 0.005-0.3 wt.-% surfactant; even more preferably 2-20 wt.-% antibody or functional fragment thereof, 5-15 wt.-% binder, 10-25 wt.-% sucrose, 0.1-2 wt.-% L-histidine and optionally 0.5-1.5 wt.-% other buffers, 0.5-1.5 wt.-% anti-tacking agent, and/or 0.05-0.3 wt.-% surfactant; even more preferably 8-12 wt.-% antibody or functional fragment thereof, 6-8 wt.-% binder (preferably hypromellose (HPMC)), 15-18 wt.-% sucrose, 0.15-1 wt.-% L-histidine and optionally 0.6-0.9 wt.-% other buffers, 0.6-0.8 wt.-% anti-tacking agent (preferably mesoporous silica), and/or 0.05-0.1 wt.-% surfactant (preferably polysorbate 80).

[45] The solid oral dosage form of any of the preceding items, wherein the sustained release layer (3) comprises, relative to the total weight of the solid oral dosage form, 1-10 wt.-% ammonio methacrylate copolymer, 0.1-5 wt.-% plasticizer, and/or 0.05-7 wt.-% anti-tacking agent; preferably 3-6 wt.-% ammonio methacrylate copolymer, 0.2-2 wt.-% plasticizer, and/or 0.1-3 wt.-% anti-tacking agent; more preferably 3.3-4.5 wt.-% ammonio methacrylate copolymer, 0.5-1.5 wt.-% plasticizer, and/or 0.2-1 wt.-% anti-tacking agent; even more preferably 3.6-4.2 wt.-% poly(ethyl acrylate, methyl methacrylate, trimethylammonioethyl methacrylate chloride) 1:2:0.1 (e.g. Eudragit® RS 30D), 0.6-1 wt.-% plasticizer (preferably triethyl citrate (TEC)) and/or 0.3-0.5 wt.-% anti-tacking agent.

[46] The solid oral dosage form of any of the preceding items, wherein the delayed release layer (4) comprises, relative to the total weight of the solid oral dosage form, 10-40 wt.-% poly(methacrylic acid, methyl methacrylate) 1:2, 0.5-10 wt.-% plasticizer, 0.1-3.5 wt.-% surfactant, and/or 0.5-10 wt.-% anti-tacking agent (preferably glycerol monostearate (GMS)); preferably 15-35 wt.-% poly(methacrylic acid, methyl methacrylate) 1:2, 1-5 wt.-% plasticizer, 0.2-2 wt.-% surfactant, and/or 1-5 wt.-% anti-tacking agent (preferably glycerol monostearate (GMS)); more preferably 18-30 wt.-% poly(methacrylic acid, methyl methacrylate) 1:2, 1.5-3 wt.-% plasticizer, 0.5-1.3 wt.-% surfactant, and/or 1.5-3 wt.-% anti-tacking agent (preferably glycerol monostearate (GMS)); even more preferably 21-26 wt.-% poly(methacrylic acid, methyl methacrylate) 1:2, 2.0-2.6 wt.-% plasticizer, 0.7-1.1 wt.-% surfactant, and/or 2.0-2.6 wt.-% anti-tacking agent (preferably glycerol monostearate (GMS)).

[47] The solid oral dosage form of any of the preceding items, comprising up to 7 wt.-%, preferably up 6 wt.-%, more preferably up to 5 wt.-%, e.g. 5 wt.-%, water in the inert core unit and/or any of the layers, relative to the total weight of the solid oral dosage form.

[48] The solid oral dosage form according to any of items 1 to 41, wherein the solid oral dosage form (relative to the total weight of the solid oral dosage form) comprises, or consists of,
a) in the inert core unit (1), 5-75 wt.-%, preferably 15-65 wt.-%, more preferably 20-60 wt.-%, even more preferably 25-30 wt.-%, microcrystalline cellulose; and/or
b) in the depot layer (2), 0.05-30 wt.-% antibody or functional fragment thereof, 0.03-30 wt.-% binder, 0.1-40 wt.-% sucrose, 0.005-5 wt.-% L-histidine (salt and/or free base) and optionally 0.001-5 wt.-% other buffers, 0.005-8 wt.-% anti-tacking agent, and/or 0.0005-2 wt.-% surfactant; preferably 0.1-20 wt.-% antibody or functional fragment thereof, 0.1-25 wt.-% binder, 0.5-35 wt.-% sucrose, 0.01-3 wt.-% L-histidine and optionally 0.01-3 wt.-% other buffers, 0.01-5 wt.-% anti-tacking agent, and/or 0.001-1 wt.-% surfactant; more preferably 0.5-20 wt.-% antibody or functional fragment thereof, 0.5-15 wt.-% binder, 1-25 wt.-% sucrose, 0.05-2 wt.-% L-histidine and optionally 0.05-1.5 wt.-% other buffers, 0.05-1.5 wt.-% anti-tacking agent, and/or 0.005-0.3 wt.-% surfactant; even more preferably 2-20 wt.-% antibody or functional fragment thereof, 5-15 wt.-% binder, 10-25 wt.-% sucrose, 0.1-2 wt.-% L-histidine and optionally 0.5-1.5 wt.-% other buffers, 0.5-1.5 wt.-% anti-tacking agent, and/or 0.05-0.3 wt.-% surfactant; even more preferably 8-12 wt.-% antibody or functional fragment thereof, 6-8 wt.-% binder (preferably hypromellose (HPMC)), 15-18 wt.-% sucrose, 0.15-1 wt.-% L-histidine and optionally 0.6-0.9 wt.-% other buffers, 0.6-0.8 wt.-% mesoporous silica, and/or 0.05-0.1 wt.-% polysorbate 80; and/or
c) in the sustained release layer (3), 1-10 wt.-% ammonio methacrylate copolymer, 0.1-5 wt.-% plasticizer, and/or 0.05-7 wt.-% anti-tacking agent; preferably 3-6 wt.-% ammonio methacrylate copolymer, 0.2-2 wt.-% plasticizer, and/or 0.1-3 wt.-% anti-tacking agent; more preferably 3.3-4.5 wt.-% ammonio methacrylate copolymer, 0.5-1.5 wt.-% plasticizer, and/or 0.2-1 wt.-% anti-tacking agent; even more preferably 3.6-4.2 wt.-% poly(ethyl acrylate, methyl methacrylate, trimethylammonioethyl methacrylate chloride) 1:2:0.1 (e.g. Eudragit® RS 30D), 0.6-1 wt.-% triethyl citrate (TEC) and/or 0.3-0.5 wt.-% mesoporous silica; and/or
d) in the delayed release layer (4), 10-40 wt.-% poly(methacrylic acid, methyl methacrylate) 1:2, 0.5-10 wt.-% plasticizer, 0.1-3.5 wt.-% surfactant, and/or 0.5-10 wt.-% anti-tacking agent (preferably glycerol monostearate (GMS)); preferably 15-35 wt.-% poly(methacrylic acid, methyl methacrylate) 1:2, 1-5 wt.-% plasticizer, 0.2-2 wt.-% surfactant, and/or 1-5 wt.-% anti-tacking agent (preferably glycerol monostearate (GMS)); more preferably 18-30 wt.-% poly(methacrylic acid, methyl methacrylate) 1:2, 1.5-3 wt.-% plasticizer, 0.5-1.3 wt.-% surfactant, and/or 1.5-3 wt.-% anti-tacking agent (preferably glycerol monostearate (GMS)); even more preferably 21-26 wt.-% poly(methacrylic acid, methyl methacrylate) 1:2, 2.0-2.6 wt.-% plasticizer, 0.7-1.1 wt.-% surfactant, and/or 2.0-2.6 wt.-% glycerol monostearate (GMS); and/or
e) up to 5 wt.-%, e.g. 5 wt.-%, water in the inert core unit and/or any of the layers.

[49] The solid oral dosage form according any of the preceding items, wherein sustained release layer (3) comprises, apart from ammonio methacrylate copolymer, no other sustained release polymer; preferably poly(ethyl acrylate, methyl methacrylate, trimethylammonioethyl methacrylate chloride) 1:2:0.1; poly(ethyl acrylate, methyl methacrylate, trimethylammonioethyl methacrylate chloride) 1:2:0.2 (e.g. Eudragit® RL 30D); or a combination thereof, and preferably no other sustained release polymer.

[50] The solid oral dosage form according any of the preceding items, wherein sustained release layer (3) comprises poly(ethyl acrylate, methyl methacrylate, trimethylammonioethyl methacrylate chloride) 1:2:0.1 and preferably no other sustained release polymer.

[51] The solid oral dosage form according any of the preceding items, wherein the plasticizer in the sustained release layer (3) is triethyl citrate (TEC), polyethylene glycol, acetyl triethyl citrate, butyl citrate, polysorbate, polypropylene glycol, dibutyl sebacate (DBS), or a combination thereof, preferably triethyl citrate (TEC).

[52] The solid oral dosage form according any of the preceding items, wherein the anti-tacking agent in the sustained release layer (3) is mesoporous silica, colloidal silica dioxide, stearic acid, magnesium stearate, glycerol monostearate (GMS), or talc, preferably mesoporous silica.

[53] The solid oral dosage form according to any of the preceding items, wherein the solid oral dosage form comprises 0.5-15 wt.-%, preferably 1-10 wt.-%, more preferably 3-5 wt.-%, even more preferably 3.5-4.5 wt.-%, even more preferably 3.8-4.1 wt.-%, e.g. 4 wt.-%, ammonio methacrylate copolymer, preferably poly(ethyl acrylate, methyl methacrylate, trimethylammonioethyl methacrylate chloride) 1:2:0.1.

[54] The solid oral dosage form according to any of the preceding items, wherein the sustained release layer (3) comprises 60-90 wt.-%, preferably 65-85 wt.-%, more preferably 70-80 wt.-%, even more preferably 72-78 wt.-%, ammonio methacrylate copolymer, preferably poly(ethyl acrylate, methyl methacrylate, trimethylammonioethyl methacrylate chloride) 1:2:0.1, relative to the total weight of the solids (i.e. not including water) in the sustained release layer (3).

[55] The solid oral dosage form according to any of the preceding items, wherein the sustained release layer (3) comprises 2-40 wt.-%, preferably 5-30 wt.-%, more preferably 15-25 wt.-%, even more preferably 17-23 wt.-%, even more preferably 20 wt.-%, plasticizer, relative to the total weight of the ammonio methacrylate copolymers in the sustained release layer (3).

[56] The solid oral dosage form according to any of the preceding items, wherein the sustained release layer (3) comprises 1-30 wt.-%, preferably 2-25 wt.-%, more preferably 5-15 wt.-%, even more preferably 8-12 wt.-%, even more preferably 10 wt.-%, anti-tacking agent, relative to the total weight of the ammonio methacrylate copolymers in the sustained release layer (3).

[57] The solid oral dosage form according to any of the preceding items, wherein the sustained release coating comprises 5-25 wt.-%, preferably 10-20 wt.-%, more preferably 12-18 wt.-%, even more preferably 13-17 wt.-%, even more preferably 14-15 wt.-%, plasticizer, relative to the total weight of the solids (i.e. not including water) in the sustained release layer (3).

[58] The solid oral dosage form according to any of the preceding items, wherein the sustained release coating comprises 5-12 wt.-%, preferably 6-9 wt.-%, more preferably 6-8.5 wt.-%, even more preferably 6.5-8.0 wt.-%, even more preferably 6.9-7.8 wt.-%, even more preferably 7.0-7.5 wt.-%, anti-tacking agent, relative to the total weight of the solids (i.e. not including water) in the sustained release layer (3).

[59] The solid oral dosage form according to any of the preceding items, wherein the sustained release layer (3) comprises no coalescence enhancer.

[60] The solid oral dosage form according to any of items 1 to 58, wherein the sustained release layer (3) comprises a coalescence enhancer.

[61] The solid oral dosage form according to item 60, wherein the coalescence enhancer is propylene glycol monolaurate (e.g. Lauroglycol™ 90).

[62] The solid oral dosage form according to any of the preceding items, wherein the weight of the sustained release layer (3), relative to combined weight of the inert core unit (1) and the depot layer (2), is 4-8 wt.-%, preferably 5-7 wt.-% more preferably about 6 wt.-%.

[63] The solid oral dosage form according to any of the preceding items, wherein the sustained release layer (3) was applied by spray coating, preferably fluidized-bed spray coating.

[64] The solid oral dosage form according to item 63, wherein the weight gain of the solid oral dosage form after sustained release coating and drying, but before applying any further layer, is 4-8 wt.-%, preferably 5-7 wt.-%, more preferably about 6 wt.-%.

[65] The solid oral dosage form according to any of the preceding items, wherein the delayed release layer (4) comprises no polymers that are not enteric polymers.

[66] The solid oral dosage form according to any of the preceding items, wherein the delayed release layer (4) comprises, apart from poly(methacrylic acid, methyl methacrylate) 1:2, poly(methyl acrylate, methyl methacrylate, methacrylic acid) 7:3:1, poly(methacrylic acid, methyl methacrylate) 1:1, or a combination thereof, no other delayed release polymers.

[67] The solid oral dosage form according to any of the preceding items, wherein the delayed release layer (4) comprises poly(methacrylic acid, methyl methacrylate) 1:2, and preferably no other delayed release polymer.

[68] The solid oral dosage form according any of the preceding items, wherein the plasticizer in the delayed release layer (4) is triethyl citrate (TEC), polyethylene glycol, acetyl triethyl citrate, butyl citrate, polypropylene glycol, dibutyl sebacate (DBS), or a combination thereof, preferably triethyl citrate (TEC).

[69] The solid oral dosage form according any of the preceding items, wherein the anti-tacking agent in the delayed release layer (4) is mesoporous silica, colloidal silica dioxide, stearic acid, magnesium stearate, glycerol monostearate (GMS), or talc, preferably glycerol monostearate (GMS).

[70] The solid oral dosage form according to any of the preceding items, wherein the delayed release layer (4) comprises a surfactant, preferably a nonionic surfactant.

[71] The solid oral dosage form according to item 66, wherein the surfactant is selected from the group consisting of polysorbate 20, polysorbate 28, polysorbate 40, polysorbate 60, polysorbate 65, polysorbate 80, polysorbate 81, polysorbate 85, poloxamer 124, poloxamer 181, poloxamer 188, poloxamer 237, poloxamer 331, poloxamer 338 and poloxamer 407, and combinations thereof.

[72] The solid oral dosage form according to item 70, wherein the surfactant is polysorbate 20, 28, 40, 60, 65, 80, 81 and 85; preferably polysorbate 80.

[73] The solid oral dosage form according to any of the preceding items, wherein the delayed release layer (4) comprises no coalescence enhancer.

[74] The solid oral dosage form according to any of the preceding items, wherein the solid oral dosage form comprises 5-40 wt.-%, preferably 10-40 wt.-%, more preferably 10-35 wt.-%, even more preferably 15-35 wt.-%, even more preferably 15-30 wt.-%, even more preferably 18-30 wt.-%, even more preferably 20-30 wt.-%, e.g. 22-25 wt.-%, poly(methacrylic acid, methyl methacrylate) 1:2, poly(methyl acrylate, methyl methacrylate, methacrylic acid) 7:3:1, poly(methacrylic acid, methyl methacrylate) 1:1, or a combination thereof (preferably poly(methacrylic acid, methyl methacrylate) 1:2).

[75] The solid oral dosage form according to any of the preceding items, wherein the delayed release layer (4) comprises 65-95 wt.-%, preferably 70-90 wt.-%, more preferably 75-85 wt.-%, e.g about 80 wt.-%, poly(methacrylic acid, methyl methacrylate) 1:2, poly(methyl acrylate, methyl methacrylate, methacrylic acid) 7:3:1, poly(methacrylic acid, methyl methacrylate) 1:1, or a combination thereof (preferably poly(methacrylic acid, methyl methacrylate) 1:2), relative to the total weight of the solids (i.e. not including solvents) in the delayed release layer (4).

[76] The solid oral dosage form according to any of the preceding items, wherein the delayed release layer (4) comprises 1-30 wt.-%, preferably 2-25 wt.-%, more preferably 5-15 wt.-%, even more preferably 8-12 wt.-%, even more preferably 10 wt.-%, plasticizer, relative to the total weight of the delayed release polymers in the delayed release layer (4).

[77] The solid oral dosage form according to any of the preceding items, wherein the delayed release layer (4) comprises 1-30 wt.-%, preferably 2-25 wt.-%, more preferably 5-15 wt.-%, even more preferably 8-12 wt.-%, even more preferably 10 wt.-%, anti-tacking agent, relative to the total weight of the delayed release polymers in the delayed release layer (4).

[78] The solid oral dosage form according to any of the preceding items, wherein the delayed release layer (4) comprises 0.1-15 wt.-%, preferably 0.5-10 wt.-%, more preferably 1-6 wt.-%, even more preferably 2-5 wt.-%, even more preferably 3-4.5 wt.-%, even more preferably 3.5-4 wt.-%, surfactant, relative to the total weight of the delayed release polymers in the delayed release layer (4).

[79] The solid oral dosage form according to any of the preceding items, wherein the delayed release layer (4) comprises 1-25 wt.-%, preferably 4-15 wt.-%, more preferably 5-12 wt.-%, even more preferably 6-10 wt.-%, even more preferably 7-9 wt.-%, even more preferably about 8 wt.-%, plasticizer, relative to the total weight of the solids (i.e. not including solvents) in the delayed release layer (4).

[80] The solid oral dosage form according to any of the preceding items, wherein the delayed release layer (4) comprises 1-25 wt.-%, preferably 4-15 wt.-%, more preferably 5-12 wt.-%, even more preferably 6-10 wt.-%, even more preferably 7-9 wt.-%, even more preferably about 8 wt.-%, even more preferably about 7.5 wt.-%, anti-tacking agent, relative to the total weight of the solids (i.e. not including solvents) in the delayed release layer (4).

[81] The solid oral dosage form according to any of the preceding items, wherein the delayed release layer (4) comprises 0.1-15 wt.-%, preferably 0.5-10 wt.-%, more preferably 1-5 wt.-%, even more preferably 2-4 wt.-%, even more preferably 2.5-3.5 wt.-%, even more preferably 3-3.5 wt.-%, even more preferably about 3-3.2 wt.-%, surfactant, relative to the total weight of the solids (i.e. not including solvents) in the delayed release layer (4).

[82] The solid oral dosage form according to any of the preceding items, wherein the solid dosage form, in the delayed release layer (4), comprises 7-13 mg/cm², preferably 8-12 mg/cm², more preferably 9-11.5 mg/cm², even more preferably about 9.5 mg/cm², or about 11.50 mg/cm², poly(methacrylic acid, methyl methacrylate) 1:2, poly(methyl acrylate, methyl methacrylate, methacrylic acid) 7:3:1, poly(methacrylic acid, methyl methacrylate) 1:1, or a combination thereof (preferably poly(methacrylic acid, methyl methacrylate) 1:2).

[83] The solid oral dosage form according to any of the preceding items, wherein the delayed release layer (4) was applied by spray coating, preferably fluidized-bed spray coating.

[84] The solid oral dosage form according to any of the preceding items, wherein the solid oral dosage form comprises an amount of the antibody or functional fragment thereof that allows the administration of a therapeutically effective dose of the antibody or functional fragment thereof as a single unit dose.

[85] The solid oral dosage form according to any of the preceding items, wherein the solid oral dosage form (relative to the total weight of the solid oral dosage form) comprises, or consists of,
a) in the inert core unit (1), 57.5-58.2 wt.-% microcrystalline cellulose; preferably 57.65-58.05 wt.-% microcrystalline cellulose (e.g. cellets 700);
b) in the depot layer (2), 0.65-0.95 wt.-% antibody or functional fragment thereof, 0.44-74 wt.-% hypromellose (HPMC) (e.g. Pharmacoat 603), 1.24-1.60 wt.-% sucrose, 0.06-0.10 wt.-% L-histidine (e.g. L-Histidine and/or L-Histidine HCL buffer salts solid), 0.04-0.08 wt.-% mesoporous silica (e.g. having average particle size of 3.4-3.6 μm and/or an average pore volume of 1.58-1.62 cm³/g, e.g. Syloid 244FP), and 0.006-0.014 wt.-% polysorbate 80; preferably 0.75-0.85 wt.-% antibody or functional fragment thereof, 0.54-64 wt.-% hypromellose (HPMC) (e.g. Pharmacoat 603), 1.34-1.50 wt.-% sucrose, 0.07-0.09 wt.-% L-histidine (e.g. L-Histidine and/or L-Histidine HCL buffer salts solid), 0.05-0.07 wt.-% mesoporous silica (e.g. having average particle size of 3.4-3.6 μm and/or an average pore volume of 1.58-1.62 cm³/g, e.g. Syloid 244FP), and 0.008-0.012 wt.-% polysorbate 80;
c) in the sustained release layer (3), 3.8-4.2 wt.-% poly(ethyl acrylate, methyl methacrylate, trimethylammonioethyl methacrylate chloride) 1:2:0.1 (e.g. Eudragit® RS solids), 0.65-0.95 wt.-% triethyl citrate (TEC) and 0.30-0.50 wt.-% mesoporous silica (e.g. having average particle size of 3.4-3.6 μm and/or an average pore volume of 1.58-1.62 cm³/g, e.g. Syloid 244FP); preferably 3.9-4.1 wt.-% poly(ethyl acrylate, methyl methacrylate, trimethylammonioethyl methacrylate chloride) 1:2:0.1 (e.g. Eudragit® RS solids), 0.75-0.85 wt.-% triethyl citrate (TEC) and 0.35-0.45 wt.-% mesoporous silica (e.g. having average particle size of 3.4-3.6 μm and/or an average pore volume of 1.58-1.62 cm³/g, e.g. Syloid 244FP);
d) in the delayed release layer (4), 23.1-23.7 wt.-% poly(methacrylic acid, methyl methacrylate) 1:2 (e.g. Eudragit® S100 solids), 2.19-2.49 wt.-% triethyl citrate (TEC), 0.79-1.09 wt.-% polysorbate 80, and 2.19-2.49 wt.-% glycerol monostearate (GMS); preferably 23.25-23.55 wt.-% poly(methacrylic acid, methyl methacrylate) 1:2 (e.g. Eudragit® S100 solids), 2.29-2.39 wt.-% triethyl citrate (TEC), 0.89-0.99 wt.-% polysorbate 80, and 2.29-2.39 wt.-% glycerol monostearate (GMS); and/or
e) 4.7 wt.-% to 5.3 wt.-% water; preferably 4.9 wt.-% to 5.1 wt.-% water in the inert core unit and/or any of the layers.

[86] The solid oral dosage form according to any of items 1-84, wherein the solid oral dosage form (relative to the total weight of the solid oral dosage form) comprises, or consists of,
a) in the inert core unit (1), 26.85-27.45 wt.-% microcrystalline cellulose; preferably 27.0-27.3 wt.-% microcrystalline cellulose (e.g. cellets 700);
b) in the depot layer (2), 9.23-9.73 wt.-% antibody or functional fragment thereof, 6.7-7.1 wt.-% hypromellose (HPMC) (e.g. Pharmacoat 603), 16.62-17.12 wt.-% sucrose, 0.79-1.09 wt.-% L-histidine (e.g. L-Histidine and/or L-Histidine HCL buffer salts solid), 0.54-0.84 wt.-% mesoporous silica (e.g. having average particle size of 3.4-3.6 μm and/or an average pore volume of 1.58-1.62 cm³/g, e.g. Syloid 244FP), and 0.06-0.10 wt.-% polysorbate 80; preferably 9.38-9.58 wt.-% antibody or functional fragment thereof, 6.8-7.0 wt.-% hypromellose (HPMC) (e.g. Pharmacoat 603), 16.72-17.02 wt.-% sucrose, 0.89-0.99 wt.-% L-histidine (e.g. L-Histidine and/or L-Histidine HCL buffer salts solid), 0.64-0.74 wt.-% mesoporous silica (e.g. having average particle size of 3.4-3.6 μm and/or an average pore volume of 1.58-1.62 cm³/g, e.g. Syloid 244FP), and 0.07-0.09 wt.-% polysorbate 80;
c) in the sustained release layer (3), 3.74-4.14 wt.-% poly(ethyl acrylate, methyl methacrylate, trimethylammonioethyl methacrylate chloride) 1:2:0.1 (e.g. Eudragit® RS solids), 0.64-0.94 wt.-% triethyl citrate (TEC) and 0.29-0.49 wt.-% mesoporous silica (e.g. having average particle size of 3.4-3.6 μm and/or an average pore volume of 1.58-1.62 cm³/g, e.g. Syloid 244FP); preferably 3.84-4.04 wt.-% poly(ethyl acrylate, methyl methacrylate, trimethylammonioethyl methacrylate chloride) 1:2:0.1 (e.g. Eudragit® RS solids), 0.74-0.84 wt.-% triethyl citrate (TEC) and 0.34-0.44 wt.-% mesoporous silica (e.g. having average particle size of 3.4-3.6 μm and/or an average pore volume of 1.58-1.62 cm³/g, e.g. Syloid 244FP);
d) in the delayed release layer (4), 22.63-23.23 wt.-% poly(methacrylic acid, methyl methacrylate) 1:2 (e.g. Eudragit® S100 solids), 2.14-2.44 wt.-% triethyl citrate (TEC), 0.77-0.107 wt.-% polysorbate 80, and 2.14-2.44 wt.-% glycerol monostearate (GMS); preferably 22.78-23.08 wt.-% poly(methacrylic acid, methyl methacrylate) 1:2 (e.g. Eudragit® S100 solids), 2.24-2.34 wt.-% triethyl citrate (TEC), 0.87-0.97 wt.-% polysorbate 80, and 2.24-2.34 wt.-% glycerol monostearate (GMS); and/or
e) 4.03 wt.-% to 4.63 wt.-% water; preferably 4.23 wt.-% to 4.43 wt.-% water in the inert core unit and/or any of the layers.

[87] The solid oral dosage form according to any of items 1-84, wherein the solid oral dosage form (relative to the total weight of the solid oral dosage form) comprises, or consists of,
a) in the inert core unit (1), 24.92-25.52 wt.-% microcrystalline cellulose, preferably 25.07-25.37 wt.-% microcrystalline cellulose (e.g. cellets 700);
b) in the depot layer (2), 9.0-9.5 wt.-% antibody or functional fragment thereof, 6.54-6.94 wt.-% hypromellose (HPMC) (e.g. Pharmacoat 603), 16.23-16.73 wt.-% sucrose, 0.76-1.06 wt.-% L-histidine (e.g. L-Histidine and/or L-Histidine HCL buffer salts solid), 0.52-0.82 wt.-% mesoporous silica (e.g. having average particle size of 3.4-3.6 μm and/or an average pore volume of 1.58-1.62 cm³/g, e.g. Syloid 244FP), and 0.06-0.10 wt.-% polysorbate 80; preferably 9.16-9.36 wt.-% antibody or functional fragment thereof, 6.64-6.84 wt.-% hypromellose (HPMC) (e.g. Pharmacoat 603), 16.33-16.63 wt.-% sucrose, 0.86-0.96 wt.-% L-histidine (e.g. L-Histidine and/or L-Histidine HCL buffer salts solid), 0.62-0.72 wt.-% mesoporous silica (e.g. having average particle size of 3.4-3.6 μm and/or an average pore volume of 1.58-1.62 cm³/g, e.g. Syloid 244FP), and 0.07-0.09 wt.-% polysorbate 80;
c) in the sustained release layer (3), 3.6-4.0 wt.-% poly(ethyl acrylate, methyl methacrylate, trimethylammonioethyl methacrylate chloride) 1:2:0.1 (e.g. Eudragit® RS solids), 0.61-0.91 wt.-% triethyl citrate (TEC) and 0.28-0.48 wt.-% mesoporous silica (e.g. having average particle size of 3.4-3.6 μm and/or an average pore volume of 1.58-1.62 cm³/g, e.g. Syloid 244FP); preferably 3.7-3.9 wt.-% poly(ethyl acrylate, methyl methacrylate, trimethylammonioethyl methacrylate chloride) 1:2:0.1 (e.g. Eudragit® RS solids), 0.71-0.81 wt.-% triethyl citrate (TEC) and 0.33-0.43 wt.-% mesoporous silica (e.g. having average particle size of 3.4-3.6 μm and/or an average pore volume of 1.58-1.62 cm³/g, e.g. Syloid 244FP);
d) in the delayed release layer (4), 24.5-25.1 wt.-% poly(methacrylic acid, methyl methacrylate) 1:2 (e.g. Eudragit® S100 solids), 2.33-2.63 wt.-% triethyl citrate (TEC), 0.85-1.15 wt.-% polysorbate 80, and 2.33-2.63 wt.-% glycerol monostearate (GMS); 24.65-24.95 wt.-% poly(methacrylic acid, methyl methacrylate) 1:2 (e.g. Eudragit® S100 solids), 2.43-2.53 wt.-% triethyl citrate (TEC), 0.94-1.04 wt.-% polysorbate 80, and 2.43-2.53 wt.-% glycerol monostearate (GMS); and/or
e) 4.65 wt.-% to 5.25 wt.-% water; preferably 4.85 wt.-% to 5.05 wt.-% water in the inert core unit and/or any of the layers.

[88] The solid oral dosage form according to any of items 1-84, wherein the solid oral dosage form (relative to the total weight of the solid oral dosage form) comprises, or consists of,
a) in the inert core unit (1), 25.06-25.66 wt.-% microcrystalline cellulose, preferably 25.21-25.51 wt.-% microcrystalline cellulose (e.g. cellets 700);
b) in the depot layer (2), 8.76-9.26 wt.-% antibody or functional fragment thereof, 6.38-6.78 wt.-% hypromellose (HPMC) (e.g. Pharmacoat 603), 15.57-16.17 wt.-% sucrose, 0.73-1.03 wt.-% L-histidine (e.g. L-Histidine and/or L-Histidine HCL buffer salts solid), 0.51-0.81 wt.-% mesoporous silica (e.g. having average particle size of 3.4-3.6 μm and/or an average pore volume of 1.58-1.62 cm³/g, e.g. Syloid 244FP), and 0.06-0.10 wt.-% polysorbate 80; preferably 8.91-9.11 wt.-% antibody or functional fragment thereof, 6.48-6.68 wt.-% hypromellose (HPMC) (e.g. Pharmacoat 603), 15.72-16.02 wt.-% sucrose, 0.83-0.93 wt.-% L-histidine (e.g. L-Histidine and/or L-Histidine HCL buffer salts solid), 0.61-0.71 wt.-% mesoporous silica (e.g. having average particle size of 3.4-3.6 μm and/or an average pore volume of 1.58-1.62 cm³/g, e.g. Syloid 244FP), and 0.07-0.09 wt.-% polysorbate 80;
c) in the sustained release layer (3), 3.48-3.88 wt.-% poly(ethyl acrylate, methyl methacrylate, trimethylammonioethyl methacrylate chloride) 1:2:0.1 (e.g. Eudragit® RS solids), 0.59-0.89 wt.-% triethyl citrate (TEC) and 0.27-0.47 wt.-% mesoporous silica (e.g. having average particle size of 3.4-3.6 μm and/or an average pore volume of 1.58-1.62 cm³/g, e.g. Syloid 244FP); preferably 3.58-3.78 wt.-% poly(ethyl acrylate, methyl methacrylate, trimethylammonioethyl methacrylate chloride) 1:2:0.1 (e.g. Eudragit® RS solids), 0.69-0.79 wt.-% triethyl citrate (TEC) and 0.32-0.42 wt.-% mesoporous silica (e.g. having average particle size of 3.4-3.6 μm and/or an average pore volume of 1.58-1.62 cm³/g, e.g. Syloid 244FP);
d) in the delayed release layer (4), 25.41-26.01 wt.-% poly(methacrylic acid, methyl methacrylate) 1:2 (e.g. Eudragit® S100 solids), 2.42-2.72 wt.-% triethyl citrate (TEC), 0.88-1.18 wt.-% polysorbate 80, and 2.42-2.72 wt.-% glycerol monostearate (GMS); 25.56-25.86 wt.-% poly(methacrylic acid, methyl methacrylate) 1:2 (e.g. Eudragit® S100 solids), 2.52-2.62 wt.-% triethyl citrate (TEC), 0.98-1.08 wt.-% polysorbate 80, and 2.52-2.62 wt.-% glycerol monostearate (GMS); and/or
e) 4.60 wt.-% to 5.2 wt.-% water; preferably 4.80 wt.-% to 5.00 wt.-% water in the inert core unit and/or any of the layers

[89] The solid oral dosage form according to any of the preceding items, wherein the functional antibody fragment is a Fab fragment, a F(ab′)2 fragment, a Fab′ fragment, an scFv, a dsFv, a VHH, a diabody, a triabody, a tetrabody, an Fc fusion protein or a minibody.

[90] The solid oral dosage form according to any of the preceding items, wherein the antibody or functional fragment thereof is selected from antibodies specific to tumor necrosis factor alpha (TNFα) and functional fragments thereof, antibodies specific to α4β7 integrin and functional fragments thereof, antibodies specific to CD3, CD4 or CD20 and functional fragments thereof, antibodies specific to interleukin 6 (IL-6), interleukin 12 (IL-12), interleukin 13 (IL-13), interleukin 23 (IL-23) or to their receptors and functional fragments thereof, antibodies specific to CXCL10/IP-10 and functional fragments thereof, and antibodies specific to p40 protein subunit and functional fragments thereof.

[91] The solid oral dosage form according to any of the preceding items, wherein the antibody or functional fragment thereof is suitable for use in the treatment of immune checkpoint inhibitor (ICPI) induced colitis, ICPI induced enterocolitis, ICPI induced diarrhoea and/or an inflammatory bowel disease (IBD), like Crohn's disease or ulcerative colitis.

[92] The solid oral dosage form according to any of the preceding items, wherein the antibody or functional fragment thereof is an antibody specific to tumor necrosis factor alpha (TNFα) or functional fragment thereof.

[93] The solid oral dosage form according to item 92, wherein the antibody specific to TNFα or functional fragment thereof is an anti-TNFα antibody.

[94] The solid oral dosage form according to any of the preceding items, wherein the antibody or functional fragment thereof is an anti-TNFα antibody or functional fragment thereof with a TNFα binding domain comprising (i) a V_{L} domain comprising a CDR1 region having an amino acid sequence as shown in SEQ ID NO:1, a CDR2 region having an amino acid sequence as shown in SEQ ID NO:2, and a CDR3 region having an amino acid sequence as shown in SEQ ID NO:3, and/or (ii) a V_{H} domain comprising a CDR1 region having an amino acid sequence as shown in SEQ ID NO:4, a CDR2 region having an amino acid sequence as shown in SEQ ID NO:5, and a CDR3 region having the amino acid sequence as shown in SEQ ID NO:6.

[95] The solid oral dosage form according to any of the preceding items, wherein the antibody or functional fragment thereof is an anti-TNFα antibody or functional fragment thereof with a TNFα binding domain comprising, or consisting of, (i) a V_{L} domain having an amino acid sequence in accordance with the amino acid sequence as shown in SEQ ID NO:8, and/or (ii) a V_{H} domain having an amino acid sequence in accordance with the amino acid sequence as shown in SEQ ID NO:7.

[96] The solid oral dosage form according to any of items 1-93, wherein the antibody or functional fragment thereof is an anti-TNFα antibody or functional fragment thereof with a TNFα binding domain comprising (i) a V_{L} domain comprising a CDR1 region having an amino acid sequence as shown in SEQ ID NO:9, a CDR2 region having an amino acid sequence as shown in SEQ ID NO:10, and a CDR3 region having an amino acid sequence as shown in SEQ ID NO:11, and/or (ii) a V_{H} domain comprising a CDR1 region having an amino acid sequence as shown in SEQ ID NO:12, a CDR2 region having an amino acid sequence as shown in SEQ ID NO:13, and a CDR3 region having the amino acid sequence as shown in SEQ ID NO:14.

[97] The solid oral dosage form according to any of items 1-93, wherein the antibody or functional fragment thereof is an anti-TNFα antibody or functional fragment thereof with a TNFα binding domain comprising, or consisting of, (i) a V_{L} domain having an amino acid sequence in accordance with the amino acid sequence as shown in SEQ ID NO:16 or in SEQ ID NO:17, and/or (ii) a V_{H} domain having an amino acid sequence in accordance with the amino acid sequence as shown in SEQ ID NO:15.

[98] The solid oral dosage form according to any of items 1-93, wherein the antibody or functional fragment thereof is an anti-TNFα antibody comprising a Fc region having, or consisting of, an amino acid sequence in accordance with the amino acid sequence as shown in SEQ ID NO:18, in SEQ ID NO:19, in SEQ ID NO:20, in SEQ ID NO:21, or in SEQ ID NO:22, preferably in SEQ ID NO:18.

[99] The solid oral dosage form according to any of items 1-93, wherein the antibody or functional fragment thereof is an anti-TNFα antibody comprising, or consisting of, (i) a light chain having, or consisting of, an amino acid sequence in accordance with the amino acid sequence as shown in SEQ ID NO:23, and (ii) a heavy chain having, or consisting of, an amino acid sequence in accordance with the amino acid sequence as shown in SEQ ID NO:26.

[100] The solid oral dosage form according to any of items 1-93, wherein the antibody or functional fragment thereof is an anti-TNFα antibody comprising, or consisting of (i) a light chain having, or consisting of, an amino acid sequence in accordance with the amino acid sequence as shown in SEQ ID NO:24 or in SEQ ID NO:25, and (ii) a heavy chain having, or consisting of, an amino acid sequence in accordance with the amino acid sequence as shown in SEQ ID NO:27.

[101] The solid oral dosage form according to any of items 1-93, wherein the antibody or functional fragment thereof is an anti-TNFα antibody comprising, or consisting of (i) a light chain having, or consisting of, an amino acid sequence in accordance with the amino acid sequence as shown in SEQ ID NO:23, and (ii) a heavy chain having, or consisting of, an amino acid sequence in accordance with the amino acid sequence as shown in SEQ ID NO:28, in SEQ ID NO:29, or in SEQ ID NO:30.

[102] The solid oral dosage form according to any of items 1-93, wherein the antibody or functional fragment thereof is an anti-TNFα antibody comprising, or consisting of (i) a light chain having, or consisting of, an amino acid sequence in accordance with the amino acid sequence as shown in SEQ ID NO:24 or in SEQ ID NO:25, and (ii) a heavy chain having, or consisting of, an amino acid sequence in accordance with the amino acid sequence as shown in SEQ ID NO:31, in SEQ ID NO:32, or in SEQ ID NO:33.

[103] The solid oral dosage form according to any of items 1-93, wherein the antibody or functional fragment thereof is an anti-TNFα antibody comprising, or consisting of (i) a light chain having, or consisting of, an amino acid sequence in accordance with the amino acid sequence as shown in SEQ ID NO:23, and (ii) a heavy chain having, or consisting of, an amino acid sequence in accordance with the amino acid sequence as shown in SEQ ID NO:34.

[104] The solid oral dosage form according to any of items 1-93, wherein the antibody or functional fragment thereof is an anti-TNFα antibody comprising, or consisting of (i) a light chain having, or consisting of, an amino acid sequence in accordance with the amino acid sequence as shown in SEQ ID NO:24 or in SEQ ID NO:25, and (ii) a heavy chain having, or consisting of, an amino acid sequence in accordance with the amino acid sequence as shown in SEQ ID NO:35.

[105] The solid oral dosage form according to any of items 1-91, wherein the antibody or functional fragment thereof is selected from infliximab, adalimumab, etanercept, certolizumab pegol, golimumab, visilizumab, eldelumab, abrilumab, canakinumab, tocilizumab, ustekinumab, natalizumab, etrolizumab, priliximab, vedolizumab and functional fragments thereof.

[106] The solid oral dosage form according to any of items 1-92, wherein the anti-TNFα antibody or functional fragment thereof is selected from infliximab, adalimumab, etanercept, certolizumab pegol and golimumab or a functional fragment thereof.

[107] The solid oral dosage form according to any of the preceding items, allowing a sustained release of the antibody or functional fragment thereof over a time period of at least 5 h, preferably at least 10 h, more preferably at least 12 h, even more preferably at least 15 h, even more preferably at least 20 h, most preferably at least 24 h, upon continuously immersing the solid oral dosage form in an aqueous solution under continuous agitation at a pH of 6.5-7.5, preferably about 6.8.

[108] The solid oral dosage form according to any of the preceding items, wherein, upon continuously immersing the solid oral dosage form in an aqueous solution under continuous agitation and at a pH of 6.5-7.5, preferably pH of 6.5-7.0, more preferably a pH of 6.8, a sustained release, preferably with a substantially constant release rate, of at least 60%, preferably of at least 80%, more preferably at least 90%, of the antibody or functional fragment thereof in the solid oral dosage form over 4-30 h, preferably 8-28 h, more preferably 16-26 h, even more preferably 24 h, is achieved.

[109] The solid oral dosage form according to any of the preceding items, wherein water content in the solid oral dosage form is less than 10 wt.-%, preferably less than 8 wt.-%, more preferably less than 5 wt.-%, or less than 3 wt.-%, less than 1.5 wt.-% or less than 1 wt.-%, relative to the total weight of the solid oral dosage.

[110] The solid oral dosage form according to any of the preceding items wherein the fraction of total content of antibody or functional fragment thereof present in the solid oral dosage form as dimers and other aggregates does not exceed 15%, preferably 12%, more preferably 10%, even more preferably 8%, even more preferably 7%, even more preferably 5%, even more preferably 3%.

[111] The solid oral dosage form according to any of the preceding items wherein the fraction of total content of antibody or functional fragment thereof present in the solid oral dosage form as fragments of the full-length antibody or functional fragment thereof does not exceed 15%, preferably 12%, more preferably 10%, even more preferably 8%, even more preferably 7%, even more preferably 5%, even more preferably 3%.

[112] The solid oral dosage form according to any of the preceding items, wherein the depot layer (2) has an average thickness of 1-300 μm, preferably 2-200 μm, more preferably 5-200 μm, even more preferably 10-200 μm, e.g. 100-200, or 150 μm.

[113] The solid oral dosage form according to any of the preceding items, wherein the sustained release layer (3) has an average thickness of 5-50 μm, preferably 10-45 μm, more preferably 15-35 μm, even more preferably 18-30 μm, even more preferably 20-30 μm, e.g. 24 or 25 μm.

[114] The solid oral dosage form according to any of the preceding items, wherein the sustained release layer (3) has a fixed average thickness of 5-50 μm, preferably 10-45 μm, more preferably 15-35 μm, even more preferably 18-30 μm, even more preferably 20-30 μm, e.g. 24 or 25 μm; preferably irrespective of the thickness the depot layer (2) and/or amount of antibody or functional fragment thereof in the depot layer (2).

[115] The solid oral dosage form according to any of the preceding items, wherein the delayed release layer (4) has an average thickness of 10-300 μm, preferably 50-180 μm, more preferably 60-160 μm, even more preferably 80-150 μm, even more preferably 90-130 μm, even more preferably 95-120 μm, e.g. 105 or 120 μm.

[116] The solid oral dosage form according to any of the preceding items, wherein the average thickness of the sustained release layer (3) is constant, irrespective of the amount of antibody or functional fragment thereof in the solid oral dosage form and/or the thickness of the depot layer.

[117] Solid oral dosage form according to any of items 1 to 116 for use in the targeted local treatment of a gastrointestinal disease, preferably immune checkpoint inhibitor (ICPI) induced colitis, ICPI induced enterocolitis, ICPI induced diarrhoea, an IBD, colorectal cancer, small intestine cancer, celiac disease, a gastrointestinal infections (e.g. Clostridium difficile infection), more preferably ICPI induced colitis, ICPI induced enterocolitis, ICPI induced diarrhoea, or an IBD.

[118] Solid oral dosage form for use according to item 117, wherein the gastrointestinal disease is IBD, preferably Crohn's disease or ulcerative colitis.

[119] Solid oral dosage form for use according to item 117, wherein the gastrointestinal disease is ICPI induced colitis, ICPI induced enterocolitis, or ICPI induced diarrhoea, preferably ICPI induced colitis.

[120] Solid oral dosage form according to any of items 117 to 119 for use in the targeted local treatment in the terminal ileum, the ileocolonic region, the ascending colon, transverse colon, the descending colon and/or the sigmoid colon of a patient, preferably a human patient.

[121] An oral multiparticulate drug delivery system, comprising a plurality of solid oral dosage forms of any one of items 1 to 116, preferably wherein the oral multiparticulate drug delivery system is a sachet/stick pack, a straw device (XStraw®), capsule, or tablet/mini-tablet, more preferably a capsule; preferably wherein the oral multiparticulate drug delivery system comprises a total amount of the antibody or functional fragment thereof suitable for oral administration to a human patient.

[122] An oral multiparticulate drug delivery system comprising a plurality of solid oral dosage forms of any of items 1 to 116, wherein each solid oral dosage form unit preferably has a predetermined axis and a predetermined cross-sectional profile, wherein at least 80% by number of those solid oral dosage forms, preferably 90%, more preferably 95%, have a median aspect ratio between 0.7 and 1.7, the aspect ratio being defined as solid oral dosage form length along the predetermined axis divided by the smallest cross-sectional dimension.

[123] The oral multiparticulate drug delivery system according to item 121 or 122, wherein the median aspect ratio is above 0.8, preferably above 0.9, and below 1.6, preferably below 1.5, more preferably 1.4, even more preferably below 1.3, even more preferably below 1.2, most preferably about 1.

[124] The oral multiparticulate drug delivery system according to any of items 121 to 123, allowing the recovery of at least 60%, preferably at least 80%, more preferably at least 85%, even more preferably at least 93%, even more preferably at least 95%, even more preferably at least 97%, even more preferably at least 98%, of the antibody or functional fragment thereof from the solid oral dosage forms.

[125] The oral multiparticulate drug delivery system according to any of items 121 to 124, allowing the recovery of at least 60%, preferably at least 80%, more preferably at least 85%, even more preferably at least 93%, even more preferably at least 95%, even more preferably at least 97%, even more preferably at least 98%, of the antibody or functional fragment thereof from the solid oral dosage forms, within 4 h, or 6 h, or 8 h, or 10 h, or 12 h, or 14 h, or 16 h, or 18 h, or 20 h, or 22 h, or 24 h, or 26 h, or 28 h, or 30 h, of continuously immersing the solid oral dosage form in an aqueous solution under continuous agitation (sustained release), at a pH of 6.5 to 7, preferably at a pH of about 6.8.

[126] The oral multiparticulate drug delivery system according to any of items 121 to 125, wherein the oral multiparticulate drug delivery system is prepared from a plurality of solid oral dosage form by compression or encapsulation, preferably encapsulation.

[127] A method for targeted local treatment of a gastrointestinal disease, comprising administering to a patient in need thereof a pharmaceutically effective amount of the solid oral dosage form of any of items 1-116 or the oral multiparticulate drug delivery system of any of items 121-126.

[128] The method of item 127, wherein the gastrointestinal disease is immune checkpoint inhibitor (ICPI) induced colitis, ICPI induced enterocolitis, ICPI induced diarrhoea, or an inflammatory bowel disease (IBD).

[129] The method of item 127 or 128, wherein the patient is a human patient and wherein optionally the solid oral dosage form or the oral multiparticulate drug delivery system is administered orally every 24-48 h, preferably every 24 h.

[130] Use of the solid dosage form of any of items 1 to 116 in the preparation of a medicament for the treatment of a gastrointestinal disease.

[131] Use according to item 130, wherein the gastrointestinal disease is IBD, preferably Crohn's disease or ulcerative colitis.

### BRIEF DESCRIPTION OF THE DRAWINGS

FIG. 1: Multi-layered solid oral dosage form of the present invention, comprising an inert core unit (1); a depot layer (2) comprising the antibody or functional fragment thereof; the sustained release layer (3); and the delayed release layer (4).

FIG. 2: Illustration showing the oral multiparticulate drug delivery system comprising the multi-layer solid oral dosage form; a description of the different layers in a single solid oral dosage form; and a scanning electron microscope (SEM) image of a final single solid oral dosage form.

FIG. 3: Dissolution kinetics for the formulations of Examples 1 to 3 (results of Example 4)

FIG. 4: Bar diagram showing the amount of antibody released after 24 hours at pH 6.8 (Example 4).

FIGS. 5A, 5B, and 5C present scanning electron microscope (SEM) images of cross-sections of the formulations of Examples 1-3. FIG. 5A corresponds to Example 1 [Eudragit S on RL]. FIG. 5B corresponds to Example 2 [Eudragit S on RS]. FIG. 5C corresponds to Example 3 [Eudragit S on EC].

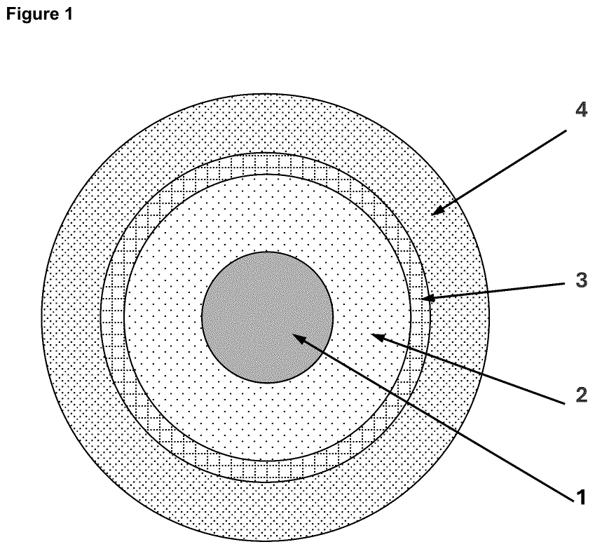

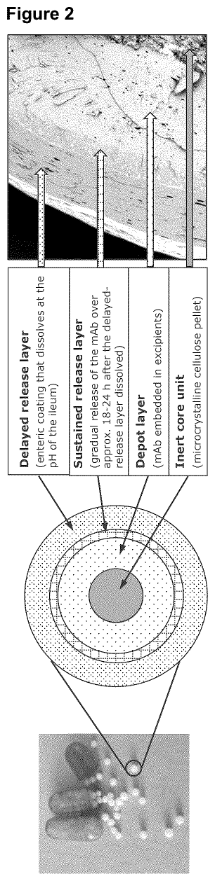

### DETAILED DESCRIPTION OF THE PREFERRED EMBODIMENTS

The present invention relates to a novel solid oral dosage form comprising i) an inert core unit (1); ii) a depot layer (2) covering the inert core unit (1) and comprising an antibody or a functional fragment thereof, preferably specific to tumor necrosis factor alpha (TNFα), as an active agent; a stabilizer, preferably sucrose; a buffer; and a polymeric binder, preferably hypromellose (HPMC); iii) a sustained release layer (3), covering the depot layer (2) and comprising an ammonio methacrylate copolymer, preferably poly(ethyl acrylate, methyl methacrylate, trimethylammonioethyl methacrylate chloride) 1:2:0.1; a plasticizer; and an anti-tacking agent; and iv) a delayed release layer (4) covering the sustained release layer (3) and comprising poly(methacrylic acid, methyl methacrylate) 1:2, poly(methyl acrylate, methyl methacrylate, methacrylic acid) 7:3:1, poly(methacrylic acid, methyl methacrylate) 1:1, or a combination thereof, preferably poly(methacrylic acid, methyl methacrylate) 1:2; an anti-tacking agent, preferably glycerol monostearate (GMS); and a plasticizer, preferably triethyl citrate (TEC).

The term “solid oral dosage form” as used herein may be understood to be equivalent to “solid oral pharmaceutical dosage form” or “pharmaceutical composition formulated into a solid oral dosage form”, is suitable for oral administration and includes for example a pellet, bead, sphere, mini-sphere, tablet, mini-tablet and the like. In one embodiment of the present invention the solid oral dosage form is a pellet, sphere, mini-sphere, bead, granule, tablet or mini-tablet. In a preferred embodiment of the present invention the solid oral dosage form is a pellet. The size of the solid oral dosage form is not particularly limited and may be characterized by a maximal diameter of between 100-4000 μm, preferably 300-3000 μm, more preferably 500-2500 μm, even more preferably 700-2000 μm, e.g. about 1200-1600 μm.

Multiple solid oral dosage forms of the present invention may be combined into a single-unit formulation in the form of an “oral multiparticulate drug delivery system”, also known as “oral multiparticulate dosage form”. The oral multiparticulate drug delivery system may be for example in the form of a hard gelatin capsule, tablet, sachet, caplet, or pill.

The term “inert core unit” as used herein is known to the skilled person. The term “inert core unit” as used herein may be understood to mean inert pellet, mini-tablet, tablet, granule, core, bead, mini-sphere or sphere, which consists of one or more of soluble or insoluble inert materials and the like, which are all pharmacologically inactive and do not interact with the active ingredient in the solid oral dosage form, that is, the antibody or functional fragment thereof.

The inert core unit may be optionally seal coated, for example to increase the strength of the core to withstand the mechanical pressures during processing.

The term “inert” as used herein preferably means that the inert core unit (1), that is including all its ingredients, is pharmacologically inactive and does not interact with the antibody or functional fragment thereof in the depot layer (2). This preferably means that the inert core unit (1) does not contain any active ingredients and does not reduce stability and activity of the antibody or functional fragment thereof in the depot layer (2) during preparation of the solid oral dosage form, its storage and later administration and dissolution.

The term “about”, as used herein, indicates the value or range of a given quantity can include quantities ranging within 10% of the stated value or range, or optionally within 5% of the value or range, or in some embodiments within 1% of the value or range. Generally, unless otherwise indicated, “wt.-%” as used herein, refers to the weight percent of a substance relative to the total weight of the solid oral dosage form. In some cases where indicated, “wt.-%” may refer to the weight percent of a substance relative to the weight of a specific layer of the solid oral dosage form.

The inert core unit (1) may be an inert pellet, mini-tablet, tablet, granule, core, bead, mini-sphere or sphere e.g. prepared from one or a mixture of excipients for example by compression or extrusion-spheronization. In a preferred embodiment the inert core unit (1) is a pellet. The pellet, for example, may comprise, or consist of, a water insoluble or water swellable material and/or may be of uniform composition. In one embodiment of the present invention, the inert core unit (1) comprises a monosaccharide, disaccharide, oligosaccharide, polysaccharide, silica, tartaric acid, calcium carbonate, or a combination thereof as a main component. In another embodiment the inert core unit (1) consists of a monosaccharide, disaccharide, oligosaccharide, polysaccharide, silica, tartaric acid, calcium carbonate, or a combination thereof.

In a specific embodiment of the present invention, the inert core unit (1), e.g. a pellet, comprises, or consists of, microcrystalline cellulose, sucrose, starch, mannitol, calcium carbonate, silica, tartaric acid, lactose, or a combination thereof, preferably microcrystalline cellulose. In a further embodiment, the inert core unit (1), e.g. a pellet, comprises microcrystalline cellulose, sucrose, starch, mannitol, calcium carbonate, silica, tartaric acid, lactose, or a combination thereof, preferably microcrystalline cellulose as a main component. “Main component” in this context refers to an inert core unit (1) comprising at least 50 wt.-%, preferably at least 70 wt.-%, more preferably at least 90 wt.-%, even more preferably at least 95 wt.-%, of said component. As used in the context of the inert core unit above, “wt.-%” refers to the weight percent of a substance relative to the weight of the inert core unit (1).

In one embodiment the inert core unit (1) is a pellet. The pellet may have a median particle size of 50-5000 μm, preferably 100-3000 μm, more preferably 200-2000 μm, even more preferably 300-1500 μm, even more preferably 400-1400, even more preferably 700-1400, even more preferably 500-1000, most preferably 500-700, 600-800, or 700-1000 μm. In another embodiment, the inert core unit (1) is a pellet with a particle size distribution such that at least 85% of the pellets have a particle size of 50-3000 μm, preferably 100-1500 μm, more preferably 350-1400 μm, even more preferably 500-1400 μm, even more preferably 700-1400, even more preferably 500-1000 μm, e.g. 500-700 μm or 700-1000 μm. According to a preferred embodiment of the present invention the inert core unit (1) is a pellet with a particle size distribution such that at least 85% of the pellets have a particle size of 700-1400 μm, preferably 700-1000 μm.

The shape of the inert core unit (1), preferably a pellet, is not particularly limited. In one embodiment, the inert core unit (1) is a pellet with a sphericity degree of at least 0.6, preferably at least 0.7, more preferably at least 0.8, even more preferably at least 0.9, even more preferably at least 0.95. In a preferred embodiment the inert core unit (1) is a pellet comprising, or consisting of, a sphere. The term “sphere” as used herein preferably refers to a particle with a sphericity degree of at least 0.8. The pellet, for example, may comprise, or consist of, a sphere, which preferably consists of a water insoluble or water swellable material and/or is of uniform composition.

The pellet or the sphere may be coated with a coating deposited on the sphere, e.g a seal coated, the seal coating preferably consisting of a water-insoluble material. According to one embodiment, the inert core unit (1) comprises, or consists of, a pellet e.g. a sphere, and optionally a seal coating covering the sphere, the seal coating consisting of a water-insoluble material.

The pellet (or the sphere) may consist of microcrystalline cellulose, sucrose, starch, mannitol, calcium carbonate, silica, tartaric acid, lactose or a combination thereof. In a preferred embodiment the inert core unit (1) is a pellet, e.g. a sphere, and consists of microcrystalline cellulose, sucrose, starch, mannitol, calcium carbonate, silica, tartaric acid, lactose or a combination thereof. In a preferred embodiment, the inert core unit (1) is a pellet, preferably in the form of a sphere, and consists of microcrystalline cellulose. Examples of commercially available inert core units include CELLETS® (Pharmatrans-Sanaq AG) and SUGLETS® sugar spheres (Colorcon® Ldt).

The solid oral dosage form comprises a depot layer (2) covering the inert core unit (1) and comprising an antibody or a functional fragment thereof as an active agent; a stabilizer; a buffer; and a polymeric binder. The term “depot layer” as used herein refers to a coating or coat, which comprises at least one active agent in the form of an antibody or functional fragment thereof, and which covers the inert core unit.

The term “cover” or “covering” as used herein in the context of a “layer” of the solid oral dosage form of the present invention means that the layer is positioned on top of the inert core unit or layer being covered, such that there is no other layer in between (i.e. between the covering layer and the inert core unit or layer being covered) or such that there is one or more additional layers in between, preferably such that there is no other layer positioned in between. Preferably, a layer of the solid oral dosage form of the present invention continuously covers the inert core unit or the layer being covered, such that there are no gaps in the covering layer. This may be particularly relevant for the sustained release layer and the delayed release layer, since otherwise the integrity of these layers may be compromised.

Preferably, the depot layer (2) is positioned on top of the inert core unit (1), such that there is no other layer in between (i.e. between the depot layer and the inert core unit). Preferably, the depot layer (2) of the solid oral dosage form of the present invention continuously covers the inert core unit (1), such that there are no gaps in the depot layer.

A specific layer can be separated from the inert core unit, or further layers of the solid oral dosage form that have been applied separately, by its distinct physicochemical properties. Consequently, the depot layer (2), the sustained release layer (3), and/or the delayed release layer (4) can be separated from the inert core unit and the further layers that have been applied separately after applying the depot layer, by its distinct physicochemical properties.

According to one embodiment of the present invention, the depot layer (2) of the solid oral dosage form of the invention has an average thickness of 1-300 μm, preferably 2-200 μm, more preferably 5-200 μm, even more preferably 10-200 μm, e.g. 100-200, or 150 μm.

According to another embodiment of the present invention, the sustained release layer (3) has an average thickness of 5-50 μm, preferably 10-45 μm, more preferably 15-35 μm, even more preferably 18-30 μm, even more preferably 20-30 μm, e.g. about 24 or 25 μm. According to another embodiment of the present invention, the thickness of the sustained release layer (3) is constant and not dependent on the amount of active agent and/or thickness of the depot layer (2). According to another embodiment, the sustained release layer (3) has a fixed average thickness of 5-50 μm, preferably 10-45 μm, more preferably 15-35 μm, even more preferably 18-30 μm, even more preferably 20-30 μm, e.g. 24 or 25 μm; preferably irrespective of the thickness the depot layer (2) and/or amount of antibody or functional fragment thereof in the depot layer (2).

According to a preferred embodiment of the present invention, the delayed release layer (4) has an average thickness of 10-300 μm, preferably 50-180 μm, more preferably 60-160 μm, even more preferably 80-150 μm, even more preferably 90-130 μm, even more preferably 95-120 μm, e.g. 105 or 120 μm.

According to a specific embodiment of the invention, the solid oral dosage form according to any of the of embodiments described above, the average thickness of the sustained release layer (3) is constant, irrespective of the amount of antibody or functional fragment thereof in the solid oral dosage form and/or the thickness of the depot layer.

The thickness of a layer may be determined using scanning electron microscopy (SEM), based on e.g. 10, or 15, or 20, or 30, or 50 different measurement points in the layer.

The term “antibody”, in the context of the present invention, refers to “immunoglobulin” (Ig), which is defined as a protein belonging to the class IgG, IgM, IgE, IgA, or IgD (or any subclass thereof), and includes all conventionally known antibodies and functional fragments thereof. The antibody or functional fragment thereof used for the present invention is an active agent, i.e. the antibody or functional fragment thereof is incorporated into the solid oral dosage form due to the pharmacological activity of the antibody or functional fragment thereof in a patient.

In the context of the present invention, a “functional fragment” of an antibody/immunoglobulin is defined as antigen-binding fragment or other derivative of a parental antibody that essentially maintains the properties of such a parental antibody. An “antigen-binding fragment” of an antibody/immunoglobulin is defined as a fragment (e.g., a variable region of an IgG) that retains the antigen-binding region. An “antigen-binding region” of an antibody typically is found in one or more hypervariable region(s) of an antibody, i.e., the CDR-1, -2, and/or -3 regions. “Antigen-binding fragments” according to the invention include the domain of a F(ab′)2 fragment and a Fab fragment. “Functional fragments” of the invention include Fab fragment, F(ab′)2 fragment, Fab′ fragment, scFv, dsFv, VHH, diabody, triabody, tetrabody, Fc fusion protein and minibody. The F(ab′)2 or Fab domain may be engineered to minimize or completely remove the intermolecular disulphide interactions that occur between the CH1 and CL domains. The antibodies or functional fragments thereof used for the present invention may be part of bi- or multifunctional constructs.

Fab fragments can be obtained as the purified digestion products after digestion of an antibody with a cysteine proteinase like papain (EC 3.4.22.2). F(ab′)2 fragments can be obtained as the purified digestion products after digestion of an antibody with pepsin (EC 3.4.23.1) or IdeS (Immunoglobulin degrading enzyme from Streptococcus pyogenes; EC 3.4.22). Fab′ fragments can be obtained from F(ab′)2 fragments in mild reducing conditions, whereby each F(ab′)2 molecule gives rise to two Fab′ fragments. An scFv is a single chain Fv fragment in which the variable light (“VL”) and variable heavy (“VH”) domains are linked by a peptide bridge.

A “diabody” is a dimer consisting of two fragments, each having variable regions joined together via a linker or the like (hereinafter referred to as diabody-forming fragments), and typically contain two VLs and two VHs. Diabody-forming fragments include those consisting of VL and VH, VL and VL, VH and VH, etc., preferably VH and VL. In diabody-forming fragments, the linker joining variable regions is not specifically limited, but preferably short enough to avoid noncovalent bonds between variable regions in the same fragment. The length of such a linker can be determined as appropriate by those skilled in the art, but typically 2-14 amino acids, preferably 3-9 amino acids, especially 4-6 amino acids are used. In this case, the VL and VH encoded on the same fragment are joined via a linker short enough to avoid noncovalent bonds between the VL and VH on the same chain and to avoid the formation of single-chain variable region fragments so that dimers with another fragment can be formed. The dimers can be formed via either covalent or noncovalent bonds or both between diabody-forming fragments.

Moreover, diabody-forming fragments can be joined via a linker or the like to form single-chain diabodies (sc(Fv)2). By joining diabody-forming fragments using a long linker of about 15-20 amino acids, noncovalent bonds can be formed between diabody-forming fragments existing on the same chain to form dimers. Based on the same principle as for preparing diabodies, polymerized antibodies such as trimers or tetramers can also be prepared by joining three or more diabody-forming fragments. In one embodiment, the functional fragment in the solid oral dosage form of the invention is a Fab fragment, a F(ab′)2 fragment, a Fab′ fragment, an scFv, a dsFv, a VHH, a diabody, a triabody, a tetrabody, an Fc fusion protein or a minibody. Preferred functional fragments used in the present invention are Fab fragments, F(ab′)2 fragments, Fab′ fragments, scFv and diabodies.

The antibody or functional fragment thereof used in the solid oral dosage form of the present invention is not particularly limited. In one embodiment, the antibody or functional fragment thereof is an antibody. In another embodiment of the present invention, the antibody or functional fragment thereof is functional fragment as defined above. The antibody or functional fragment thereof may further comprise one or more modifications, e.g. in the form of added or substituted residues, that improve stability, specificity or targeting. These may include any such modifications that are known in the art.

The antigen against which the antibody or functional fragment is directed i.e. the immunogen, peptide, protein, or other molecular structure to which the antibody or functional fragment thereof can specifically bind, is not limited. The terms “specific to”, “specifically bind” or “specific binding” in this context are known to the skilled person. In its most general form (and when no defined reference is mentioned), “specific to” or “specific binding” refers to the ability of the antibody or functional fragment thereof to discriminate between the target of interest and an unrelated biomolecule (e.g. for antibodies specific to human TNFα to discriminate between human TNFα and an unrelated biomolecule), as determined, for example, in accordance with specificity assay methods known in the art. Such methods comprise, but are not limited to, Western blots and enzyme-linked immunosorbent assay (ELISA) tests. For example, a standard ELISA assay can be carried out. Typically, determination of binding specificity is performed by using not a single reference biomolecule, but a set of about three to five unrelated biomolecules, such as milk powder, BSA, transferrin or the like.

In one embodiment of the present invention the antibody or functional fragment thereof is suitable for use in the treatment of an inflammatory condition in the GI tract such as immune checkpoint inhibitor (ICPI) induced colitis, ICPI induced enterocolitis, ICPI induced diarrhoea, or inflammatory bowel disease (IBD, e.g. Crohn's disease or ulcerative colitis). In a further embodiment of the present invention the antibody or functional fragment thereof is suitable for use in the local treatment in the (terminal) ileum and/or large intestine of the gastrointestinal tract of a patient, in particular at the mucosa.

In a further embodiment of the present invention the antibody or functional fragment thereof is selected from antibodies specific to tumor necrosis factor alpha (TNFα) and functional fragments thereof, antibodies specific to α4β7 integrin and functional fragments thereof, antibodies specific to CD3, CD4 or CD20 and functional fragments thereof, antibodies specific to interleukin 6 (IL-6), interleukin 12 (IL-12), interleukin 13 (IL-13), interleukin 23 (IL-23) or to their receptors and functional fragments thereof, antibodies specific to CXCL10/IP-10 and functional fragments thereof, and antibodies specific to p40 protein subunit and functional fragments thereof. In yet another embodiment of the present invention the antibody or functional fragment thereof is selected from infliximab, adalimumab, etanercept, certolizumab pegol, golimumab, visilizumab, eldelumab, abrilumab, canakinumab, tocilizumab, ustekinumab, natalizumab, etrolizumab, priliximab, vedolizumab and functional fragments thereof.

In a preferred embodiment of the solid oral dosage form of the present invention, the antibody or functional fragment thereof is an antibody specific to tumor necrosis factor alpha (TNFα) or a functional fragment thereof (that is, an anti-TNFα antibody or functional fragment thereof). The antibody specific to TNFα or functional fragment thereof is not particularly limited. In one embodiment, the antibody specific to TNFα or functional fragment thereof is an antibody. In another embodiment, the antibody specific to TNFα or functional fragment thereof is functional fragment as defined above. The antibody specific to TNFα or functional fragment thereof may further comprise one or more modifications, e.g. in the form of added or substituted residues, that improve stability, specificity or targeting. These may include any such modifications that are known in the art.

The terms “anti-TNFα antibody”, “TNFα antibody” and “antibody specific to TNFα” as used herein are interchangeable, and preferably mean that the anti-TNFα antibody is able to specifically bind to TNFα, that is it preferably refers to the ability of the antibody to specifically bind to TNFα, more preferably human TNFα (e.g. identifiable by the uniport identifier P01375). The terms “specific to”, “specifically bind” or “specific binding” in this context are known to the skilled person (see above). In one embodiment, “specific binding” in the context of TNFα refers inter alia to the ability of the antibody or fragment to discriminate between human TNFα and human TNFβ.

In one embodiment of the present invention the anti-TNFα antibody or functional fragment thereof is suitable for use in the treatment of immune checkpoint inhibitor (ICPI) induced colitis, ICPI induced enterocolitis, ICPI induced diarrhoea, and/or an inflammatory bowel disease (IBD), e.g. Crohn's disease or ulcerative colitis. In another embodiment of the present invention the antibody or functional fragment thereof is suitable for use in the local treatment in the ileum (preferably in the terminal ileum) or large intestine of the gastrointestinal tract of a patient, in particular at the mucosa. In a preferred embodiment of the present invention, the anti-TNFα antibody or functional fragment thereof is an afucosylated antibody, meaning it is devoid of the core fucose.

Several monoclonal antibodies against TNFα (that is specific to TNFα) have been described in the prior art. Meager et al. (Hybridoma, 6, 305-311, 1987) describe murine monoclonal antibodies against recombinant TNFα. Fendly et al. (Hybridoma, 6, 359-370, 1987) describe the use of murine monoclonal antibodies against recombinant TNFα in defining neutralizing epitopes on TNF. Furthermore, in international patent application WO 92/11383, recombinant antibodies, including CDR-grafted antibodies, specific for TNFα are disclosed. U.S. Pat. No. 5,919,452 discloses anti-TNFα chimeric antibodies and their use in treating pathologies associated with the presence of TNFα. Further anti-TNFα antibodies are disclosed in Stephens et al. (Immunology, 85, 668-674, 1995), GB-A-2 246 570, GB-A-2 297 145, U.S. Pat. No. 8,673,310, US 2014/0193400, EP 2 390 267 B1, U.S. Pat. Nos. 8,293,235, 8,697,074, WO 2009/155723 A2 and WO 2006/131013 A2.

Currently approved anti-TNFα antibodies include (i) infliximab, a chimeric IgG anti-human monoclonal antibody (Remicade®); (ii) etanercept, a TNFR2 dimeric fusion protein, with an IgG1 Fc (Enbrel®); (iii) adalimumab, a fully human monoclonal antibody (mAb) (Humira®), (iv) certolizumab, a PEGylated Fab fragment (Cimzia®) and (v) golimumab, a human IgGIK monoclonal antibody (Simponi®). Moreover, various biosimilars are in development. Therefore, in some embodiments of the present invention, the anti-TNFα antibody or functional fragment thereof is selected from infliximab, adalimumab, etanercept, certolizumab pegol and golimumab or a functional fragment thereof.

In a preferred embodiment of the present invention, the antibody or functional fragment thereof is an anti-TNFα antibody or functional fragment thereof with a TNFα binding domain comprising (i) a V_{L} domain comprising a CDR1 region having an amino acid sequence as shown in SEQ ID NO:1, a CDR2 region having an amino acid sequence as shown in SEQ ID NO:2, and a CDR3 region having an amino acid sequence as shown in SEQ ID NO:3, and/or (ii) a V_{H} domain comprising a CDR1 region having an amino acid sequence as shown in SEQ ID NO:4, a CDR2 region having an amino acid sequence as shown in SEQ ID NO:5, and a CDR3 region having the amino acid sequence as shown in SEQ ID NO:6.

In another preferred embodiment of the present invention, the antibody or functional fragment thereof is an anti-TNFα antibody or functional fragment thereof with a TNFα binding domain comprising, or consisting of, (i) a V_{L} domain having, or consisting of, an amino acid sequence in accordance with the amino acid sequence as shown in SEQ ID NO:8, and/or (ii) a V_{H} domain having, or consisting of, an amino acid sequence in accordance with the amino acid sequence as shown in SEQ ID NO:7.

In yet another preferred embodiment of the present invention, the antibody or functional fragment thereof is an anti-TNFα antibody or functional fragment thereof with a TNFα binding domain comprising (i) a V_{L} domain comprising a CDR1 region having an amino acid sequence as shown in SEQ ID NO:9, a CDR2 region having an amino acid sequence as shown in SEQ ID NO:10, and a CDR3 region having an amino acid sequence as shown in SEQ ID NO:11, and/or (ii) a V_{H} domain comprising a CDR1 region having an amino acid sequence as shown in SEQ ID NO:12, a CDR2 region having an amino acid sequence as shown in SEQ ID NO:13, and a CDR3 region having the amino acid sequence as shown in SEQ ID NO:14.

In yet another preferred embodiment of the present invention, the antibody or functional fragment thereof is an anti-TNFα antibody or functional fragment thereof with a TNFα binding domain comprising, or consisting of, (i) a V_{L} domain having, or consisting of, an amino acid sequence in accordance with the amino acid sequence as shown in SEQ ID NO:16 or in SEQ ID NO:17, and/or (ii) a V_{H} domain having, or consisting of, an amino acid sequence in accordance with the amino acid sequence as shown in SEQ ID NO:15.

In yet another preferred embodiment of the present invention, the antibody or functional fragment thereof is an anti-TNFα antibody comprising a Fc region having, or consisting of, an amino acid sequence in accordance with the amino acid sequence as shown in SEQ ID NO:18, in SEQ ID NO:19, in SEQ ID NO:20, in SEQ ID NO:21, or in SEQ ID NO:22, preferably in SEQ ID NO:18.

In yet another preferred embodiment of the present invention, the antibody or functional fragment thereof is an anti-TNFα antibody comprising, or consisting of, (i) a light chain having, or consisting of, an amino acid sequence in accordance with the amino acid sequence as shown in SEQ ID NO:23, and (ii) a heavy chain having, or consisting of, an amino acid sequence in accordance with the amino acid sequence as shown in SEQ ID NO:26.

In yet another preferred embodiment of the present invention, the antibody or functional fragment thereof is an anti-TNFα antibody comprising, or consisting of (i) a light chain having, or consisting of, an amino acid sequence in accordance with the amino acid sequence as shown in SEQ ID NO:24 or in SEQ ID NO:25, and (ii) a heavy chain having, or consisting of, an amino acid sequence in accordance with the amino acid sequence as shown in SEQ ID NO:27.

In yet another preferred embodiment of the present invention, the antibody or functional fragment thereof is an anti-TNFα antibody comprising, or consisting of, (i) a light chain having, or consisting of, an amino acid sequence in accordance with the amino acid sequence as shown in SEQ ID NO:23, and (ii) a heavy chain having, or consisting of, an amino acid sequence in accordance with the amino acid sequence as shown in SEQ ID NO:28, in SEQ ID NO:29, or in SEQ ID NO:30.

In yet another preferred embodiment of the present invention, the antibody or functional fragment thereof is an anti-TNFα antibody comprising, or consisting of, (i) a light chain having, or consisting of, an amino acid sequence in accordance with the amino acid sequence as shown in SEQ ID NO:24 or in SEQ ID NO:25, and (ii) a heavy chain having, or consisting of, an amino acid sequence in accordance with the amino acid sequence as shown in SEQ ID NO:31, in SEQ ID NO:32, or in SEQ ID NO:33.

In yet another preferred embodiment of the present invention, the antibody or functional fragment thereof is an anti-TNFα antibody comprising, or consisting of, (i) a light chain having, or consisting of, an amino acid sequence in accordance with the amino acid sequence as shown in SEQ ID NO:23, and (ii) a heavy chain having, or consisting of, an amino acid sequence in accordance with the amino acid sequence as shown in SEQ ID NO:34.

In yet another preferred embodiment of the present invention, the antibody or functional fragment thereof is an anti-TNFα antibody comprising, or consisting of, (i) a light chain having, or consisting of, an amino acid sequence in accordance with the amino acid sequence as shown in SEQ ID NO:24 or in SEQ ID NO:25, and (ii) a heavy chain having, or consisting of, an amino acid sequence in accordance with the amino acid sequence as shown in SEQ ID NO:35.

The amount of the antibody or functional fragment thereof is not particularly limited. Preferably, the depot layer (2) comprises an amount of the antibody or functional fragment thereof that allows the administration of a therapeutically effective dose of the antibody or functional fragment thereof as a single unit dose (e.g. in the form of a multiparticulate drug delivery system comprising a plurality of the solid oral dosage forms of the present invention. In one embodiment, the depot layer (2) comprises 0.2-75 wt.-%, preferably 1-45 wt.-%, more preferably 10-35 wt.-%, even more preferably 20-35 wt.-%, even more preferably 25-35 wt.-% antibody or functional fragment thereof preferably specific to TNFα, relative to the total weight of the solids (i.e. not including solvents, preferably water) in the depot layer (2). According to another preferred embodiment, the solid oral dosage form of the present invention comprises 0.05-30 wt.-%, preferably 0.1-25 wt.-%, more preferably 0.1-20 wt.-%, even more preferably 0.2-20 wt.-%, even more preferably 0.2-15 wt.-%, even more preferably 0.5-12 wt.-%, even more preferably 0.5-10 wt.-%, even more preferably 0.8-12 wt.-%, even more preferably 8-12 wt.-%, even more preferably 8-10 wt.-%, e.g. about 9 wt.-%, or about 9.25 wt.-%, or about 9.5 wt.-%, of the antibody or functional fragment thereof preferably specific to TNFα.

In one embodiment of the present invention, the solid oral dosage form of the present invention comprises an amount of the antibody or functional fragment thereof that allows the oral administration of a therapeutically effective dose of the antibody or functional fragment thereof as a single unit dose, for example in the form of a tablet or preferably a capsule comprising multiple solid oral dosage forms (e.g. in the form of multiple pellets). A “therapeutically effective dose” is the amount of the at least one antibody or functional fragment thereof required to provide the desired therapeutic effect. The exact amount may vary for different antibodies or functional fragments thereof and/or for individual patients, but can be determined by one skilled in the art.

The depot layer (2) comprises a stabilizer. The nature of the stabilizer is not particularly limited and includes all stabilizers ensuring stability and activity of antibodies and functional fragments thereof, e.g. antibodies and functional fragments thereof specific to TNFα, while not compromising the integrity of the depot layer (2). In one embodiment of the present invention, the stabilizer is selected from sucrose, maltose, lactose, trehalose, glycerol, maltitol, isomalt, mannitol, sorbitol, xylitol and combinations thereof, preferably sucrose, maltose, lactose, glycerol, maltitol, isomalt, and combinations thereof. According to a preferred embodiment the stabilizer in the depot layer (2) is sucrose. The amount of stabilizer in the depot layer (2) is not particularly limited as long as it ensures stability of low and high concentration of antibody or functional fragment thereof, while not compromising the integrity of the depot layer. In one embodiment of the present invention, the depot layer (2) comprises 2-80 wt.-%, preferably 10-75 wt.-%, more preferably 20-70 wt.-%, even more preferably 30-60 wt.-%, even more preferably 35-60 wt.-%, even more preferably about 40-55 wt.-%, stabilizer (preferably sucrose), relative to the total weight of the solids (i.e. not including solvents, preferably water) in the depot layer (2). In another embodiment the weight ratio of antibody or functional fragment thereof to stabilizer (e.g. sucrose) is 1:2 to 1:1.5, preferably 1:9 to 1:1.7, e.g. about 1:1.77.

The depot layer (2) comprises a buffer. The nature of the buffer is not particularly limited and includes all buffers that ensure stability and activity of antibodies and functional fragments thereof, while not compromising the integrity of the depot layer (2). In one embodiment of the present invention, the buffer (i.e. buffer salt or salts and/or free base) in the depot layer (2) is selected from the group consisting of L-histidine buffer, citrate buffer, hydroxymethylaminomethane (TRIS) buffer, succinate buffer, phosphate buffer, acetate buffer, or salts thereof, and combinations thereof; preferably L-histidine buffer, citrate buffer, TRIS buffer, and combinations thereof; more preferably L-histidine buffer, citrate-TRIS buffer, and a combination thereof. According to a preferred embodiment the buffer (i.e. buffer salt and/or free base) in the depot layer (2) comprises, or consists of, L-histidine, preferably L-histidine monohydrochloride and/or free base.

The buffer may be present in the depot layer (2) in any amount that ensures stability and activity of antibodies and functional fragments thereof during and after preparation of the solid oral dosage form, while not compromising the integrity of the depot layer (2). In one embodiment of the present invention, the depot layer (2) comprises 0.1-10 wt.-%, preferably 0.2-6 wt.-%, more preferably, 0.3-5 wt.-%, more preferably 0.5-4.5 wt.-%, even more preferably 1-4 wt.-%, more preferably about 2-3.5 wt.-%, buffer, relative to the total weight of the solids (i.e. not including solvents, preferably water) in the depot layer (2). In a further embodiment of the present invention, the depot layer (2) comprises 0.1-10 wt.-%, preferably 0.2-5 wt.-%, more preferably 0.3-4 wt.-%, even more preferably 0.35-3.5 wt.-%, more preferably about 0.4-3 wt.-%, L-histidine salt and/or free base, and optionally 0.3-6 wt.-%, preferably 0.5-4 wt.-%, more preferably 1-3.5 wt.-%, even more preferably 1.5-3 wt.-%, more preferably about 1.5-3.5 wt.-%, other buffer salts, relative to the total weight of the solids (i.e. not including solvents, preferably water) in the depot layer (2). In another embodiment the weight ratio of antibody or functional fragment thereof to buffer, preferably L-histidine buffer (salt and/or free base), is 100:1 to 1:1, preferably 50:1 to 3:1, more preferably 30:1 to 5:1; even more preferably 15:1 to 7:1; e.g. about 10:1. In another embodiment the weight ratio of antibody or functional fragment thereof to L-histidine buffer is 100:1 to 20:1, preferably 80:1 to 40:1, more preferably 60:1 to 50:1, and optionally the weight ratio of antibody or functional fragment thereof to other buffers is 25:1 to 5:1, preferably 16:1 to 10:1, more preferably 14:1 to 12:1.

Polymeric binders (also referred to herein as “binders”) suitable for the depot layer (2) of the solid oral dosage form of the present invention comprise hypromellose (HPMC); methylcellulose (MC); polyvinylpirrolidone (PVP); hydroxypropyl cellulose (HPC); macrogol poly(vinylalcohol) grafted copolymer (e.g. Kollidon® IR); and combinations thereof; preferably hypromellose or MC. In a preferred embodiment of the present invention, the polymeric binder in the depot layer (2) of the solid oral dosage form is hypromellose. Hypromellose is also known as hydroxypropyl methylcellulose (HPMC) and is available in different at different degrees of substitution (DS), molar substitution (MS) and viscosities. According to a preferred embodiment of the present invention, the polymeric binder in the depot layer (2) is hypromellose 2910, preferably hypromellose 2910 2.6-3.6 mPas, more preferably hypromellose 2910 3 mPas.

The amount of polymeric binder in the depot layer (2) is not particularly limited as long as a stable depot layer can be easily formed. In one embodiment of the present invention, the depot layer (2) comprises 0.5-65 wt.-%, preferably 2-35 wt.-%, more preferably 10-30 wt.-%, even more preferably 15-25 wt.-%, e.g. about 20 wt.-%, polymeric binder (preferably hypromellose), relative to the total weight of the solids (i.e. not including solvents, preferably water) in the depot layer (2). In another embodiment the weight ratio of antibody or functional fragment thereof to polymeric binder (e.g. hypromellose) is 1.2:1 to 1:1.5, preferably 1.3:1 to 1:1.4, e.g. about 1.35:1.

Preferably the polymeric binder in the depot layer (2) is suitable for an immediate release drug coating. Thus, the depot layer (2), without any additional layer (that is the sustained and delayed release layers) gives rise to an immediate release of the antibody or functional fragment thereof from the dosage form. As used herein, the term “immediate release” is meant to describe a depot layer in which more than 60%, preferably more than 70%, more preferably more than 80%, even more preferably more than 90%, most preferably 95%, of the antibody or functional fragment thereof is released from the depot layer (2) after 2 h, preferably after 1 h, even more preferably after 0.5 h, of exposure to an aqueous environment. The term an “aqueous environment” as used in the context of the present invention may refer to a solution or suspension of which a large part is water. This includes intestinal fluid.

To measure the amount of antibody or functional fragment thereof released from a depot layer (2) as the outermost coating of a solid dosage form into an aqueous solution, the depot layer (drug layer) deposited on an inert core can be immersed in a defined volume of aqueous solution (preferably buffered) for a defined period of the time under continuous agitation of the aqueous (preferably buffered) solution and the resulting concentration of the at least one antibody or functional fragment thereof in the aqueous solution can be determined and compared to the initial amount applied during the layering process, considering the process efficiency and weight gain. Similarly, the release from a solid dosage form with additional coatings deposited onto the depot layer (2), e.g. a sustained release coating and/or a delayed release coating, can be determined. Means to determine an antibody concentration in an aqueous solution are known in the art and include for example measuring the absorbance at 280 nm or using a colorimetric, reagent-based protein assay like the Bradford assay or by ELISA.

According to one embodiment of the present invention, the depot layer (2) of the solid oral dosage form of the present invention comprises an anti-tacking agent. The anti-tacking agent that may be used in the depot layer (2) is not particularly limited. In one embodiment of the present invention, the anti-tacking agent in the depot layer (2) is selected from colloidal silica dioxide, mesoporous silica, glycerolmonostearate (GMS), stearic acid, magnesium stearate and talc, preferably mesoporous silica or GMS, more preferably mesoporous silica. The amount of anti-tacking agent in the depot layer (2) is not particularly limited. In one embodiment of the present invention, the depot layer (2) comprises 0.2-15 wt.-%, preferably 0.5-10 wt.-%, more preferably 1-5 wt.-%, even more preferably 1.5-3 wt.-%, even more preferably 1.5-2.5 wt.-%, e.g. about 2 wt.-%, anti-tacking agent, relative to the total weight of the solids (i.e. not including solvents, preferably water) in the depot layer (2). In another embodiment the weight ratio of anti-tacking agent to polymeric binder (e.g. hypromellose) in the depot layer (2) is 1:15 to 1:5, preferably 1:12 to 1:8, e.g. about 1:10.

According to a preferred embodiment of the present invention, the anti-tacking agent in the depot layer (2) is mesoporous silica, preferably mesoporous silica with an average particle size of 1-10 μm, preferably of 2-5 μm, more preferably 3-4 μm, even more preferably about 3.5 μm and/or an average pore volume of 1.50-1.70 cm³/g, preferably 1.55-1.65 cm³/g, more preferably 1.58-1.62 cm³/g, even more preferably about 1.60 cm³/g.

According to a further embodiment, the depot layer (2) of the solid oral dosage form of the invention does not comprise a plasticizer. According to another embodiment, the depot layer (2) of the solid oral dosage form of the invention comprises a coalescence enhancer, e.g. propylene glycol monolaurate (e.g. Lauroglycol™ 90).

According to yet another embodiment of the present invention, the depot layer (2) of the solid oral dosage form of the invention comprises a surfactant. The surfactant in the depot layer (2) is not particularly limited, however preferably it is a nonionic surfactant. According to one embodiment of the present invention, the surfactant in the depot layer (2) is selected from the group consisting of polysorbate 20, polysorbate 28, polysorbate 40, polysorbate 60, polysorbate 65, polysorbate 80, polysorbate 81, polysorbate 85, poloxamer 124, poloxamer 181, poloxamer 188, poloxamer 237, poloxamer 331, poloxamer 338 and poloxamer 407, and combinations thereof.

According to a preferred embodiment of the solid oral dosage form, the surfactant in the depot layer (2) is polysorbate 20, 28, 40, 60, 65, 80, 81 and 85. In a particularly preferred embodiment, the surfactant in the depot layer (2) is polysorbate 80. In one embodiment of the present invention, the depot layer (2) comprises 0.01-2 wt.-%, preferably 0.02-1 wt.-%, more preferably 0.03-0.8 wt.-%, even more preferably 0.05-0.5 wt.-%, even more preferably 0.1-0.3 wt.-%, even more preferably 0.2-0.3 wt.-%, surfactant, relative to the total weight of the solids (i.e. not including solvents, preferably water) in the depot layer (2).

The depot layer (2) may optionally comprise at least one further excipient. The term “excipient,” as used herein, refers to a non-therapeutic agent added to the formulation to provide for example a desired consistency, viscosity, or stabilizing effect. According to one embodiment of the present invention the at least one further excipient is selected from pharmaceutically acceptable excipients like antioxidants, humectants, protective colloids, dyes, protease inhibitors, and combinations thereof. In another embodiment, the depot layer (2) does not comprise any further excipients.

According to a preferred embodiment of the present invention, the depot layer (2) of the solid oral dosage form of the invention comprises, or consists of, antibody or functional fragment thereof e.g. specific to TNFα, as an active agent; stabilizers, preferably sucrose; buffer, preferably L-histidine, more preferably L-histidine monohydrochloride and/or free base; polymeric binder, preferably hypromellose (HPMC), more preferably hypromellose 2910 2.6-3.6 mPas; anti-tacking agent, preferably mesoporous silica; surfactant, preferably polysorbate 80; optionally further buffers like citrate-TRIS; and optionally residual solvent (preferably water). According to another preferred embodiment of the present invention, the depot layer (2) of the solid oral dosage form of the invention comprises, or consists of, antibody or functional fragment thereof e.g. specific to specific to TNFα, as an active agent; sucrose; L-histidine, preferably L-histidine monohydrochloride, and/or L-histidine free base; Hypromellose (HPMC), preferably hypromellose 2910 2.6-3.6 mPas; mesoporous silica; polysorbate 80; and optionally residual water.

According to another preferred embodiment of the present invention, the depot layer (2) of the solid oral dosage form of the invention comprises, or consists, of 0.2-75 wt.-% antibody or functional fragment thereof, 0.5-65 wt.-% binder, 2-80 wt.-% sucrose, 0.1-5 wt.-% L-histidine (salt and/or free base), 0.1-10 wt.-% other buffer s, 0.2-15 wt.-% anti-tacking agent, 0.01-1 wt.-% surfactant, and/or up to 10 wt.-% water, relative to the total weight of the depot layer; preferably 1-45 wt.-% antibody or functional fragment thereof, 2-35 wt.-% binder, 10-70 wt.-% sucrose, 0.2-2 wt.-% L-histidine, 0.5-4 wt.-% other buffers, 1-5 wt.-% anti-tacking agent, 0.05-0.5 wt.-% surfactant, and/or up to 7 wt.-% water, relative to the total weight of the depot layer; more preferably 10-35 wt.-% antibody or functional fragment thereof, 10-30 wt.-% binder, 30-60 wt.-% sucrose, 0.3-0.8 wt.-% L-histidine, 1.5-3 wt.-% other buffers, 1.5-3 wt.-% anti-tacking agent, 0.1-0.3 wt.-% surfactant, and/or up to 6 wt.-% water, relative to the total weight of the depot layer; even more preferably 25-35 wt.-% antibody or functional fragment thereof, 15-25 wt.-% binder, 40-55 wt.-% sucrose, 0.4-0.6 wt.-% L-histidine, 1.5-2.5 wt.-% other buffers, 1.5-2.5 wt.-% anti-tacking agent, 0.2-0.3 wt.-% surfactant, and/or up to 5 wt.-% water, relative to the total weight of the depot layer (2).

In the solid oral dosage of the present invention, the depot layer (2) can be deposited on the inert core unit (1) by drug layering, preferably using spray coating, more preferably fluidized-bed spray coating. Drug layering using solution/suspension layering allows the preparation of solid dosage forms with uniform size distribution and smooth surface morphology. Solution/suspension layering (or coating) involves depositing a substance dissolved or dispersed in a solvent on the surface of a substrate. An example of spray coating is fluidized-bed spray coating/air-suspension coating. In fluidized-bed spray coating one or more substances are dissolved or dispersed in a liquid carrier in the form of a solvent. This solution or dispersion is then sprayed onto a substrate, e.g. an inert core (sucrose or microcrystalline cellulose spheres, etc.) suspended in a fluidized bed of a fluidized-bed spray coater. Method for drug layering of a layer containing an antibody or functional fragment as an active ingredient onto an inert core are known in the art. Such a method can be found for example in WO 2019/057562. Methods for applying a functional coating e.g. by fluidized-bed spray coating are disclosed for example in WO 2004/062577, EP 1 684 729, WO 2005/046561 or WO2005/115340.

The inert core unit may be covered with the depot layer (2) using spray coating. For example, a fluidized-bed spray coater or a pan coater can be used. According to a preferred embodiment a fluidized-bed spray coater is used. The use of a fluidized-bed spray coater for spray coating an inert core unit is known in the art. It is beneficial for the at least one antibody or functional fragment thereof in the solid oral dosage form prepared by the inventive method, if parameters and conditions used during spray coating are carefully controlled. The fluidized-bed spray coater that may be used is not particularly limited. Fluidized-bed spray coater are known in the art and include for example fluidized-bed equipment developed and commercialized by GEA Group, Glatt GmbH, Freund-Vector Corporation and Inora Pharmaceutical Machinery Co. The depot layer coating liquid comprising the antibody or functional fragment thereof may be sprayed onto the inert core unit using a bottom-spray fluidized-bed spray coater. Alternatively, the depot layer coating liquid may be sprayed onto the inert core unit using a top-spray fluidized-bed spray coater.

It is to be understood that throughout the present disclosure, whenever referring to dissolution of, or recovery of antibodies or functional fragments thereof from, a solid dosage form/multiparticulate drug delivery system (as in the section just above and below), e.g. by continuously immersing a solid dosage form/multiparticulate drug delivery system in an aqueous (buffered) solution under continuous (constant) agitation, for example the following standard testing setup, or a related standard testing setup known to the person skilled in the art, can be used: The release of the at least one antibody or functional fragment thereof can be evaluated using a standard dissolution apparatus I (baskets), II (paddle), III (reciprocating cylinder) or apparatus IV (flow through cell), where the buffer (i.e. aqueous buffer solution) is equilibrated at 37° C. The buffer volume used for dissolution testing can be adapted for instance using mini-vessels in the apparatus I or II to allow reduction of volume required and to be more bio-relevant. Release of antibodies or functional fragments thereof during dissolution can be quantified offline by an ELISA method.

The solid oral dosage form of the present invention comprises a sustained release layer (3), covering the depot layer (2) and comprising an ammonio methacrylate copolymer, preferably poly(ethyl acrylate, methyl methacrylate, trimethylammonioethyl methacrylate chloride) 1:2:0.1; a plasticizer; and an anti-tacking agent. The term “sustained release” is known in the art. As used herein, the term “sustained release” may be used to describe a continuous release of the antibody or functional fragment thereof from the solid oral dosage form over several hours, e.g. at a constant or nearly constant release rate, e.g. when immersed in an aqueous (buffered) solution under continuous agitation. According to one embodiment, the term “sustained release” is used to describe a continuous release of the antibody or functional fragment thereof from the solid oral dosage form, preferably at a nearly constant release rate, over several hours in a suitable aqueous environment. According to another embodiment, the term “sustained release” is used to describe a release of the antibody or functional fragment thereof from the solid oral dosage form, such that a substantial fraction of antibody or functional fragment thereof is release from the solid oral dosage form upon exposure to an aqueous environment over a prolonged time period, e.g. over at least 6 h, preferably at least 10 h, more preferably at least 14 h, even more preferably at least 18 h, e.g. at least 24 h.

The term “cover” or “covering” as used in this context is to be understood as defined above. Preferably, the sustained release layer (3) is positioned on top of the depot layer (2) being covered, such that there is no other layer in between (i.e. between the sustained release layer and the depot layer). Preferably, the sustained release layer of the solid oral dosage form of the present invention continuously covers the depot layer, such that there are no gaps in the sustained release layer (3).

The term “ammonio methacrylate copolymer” is known in the art. For example, the term “ammonio methacrylate copolymer” may refer to poly(ethyl acrylate, methyl methacrylate, trimethylammonioethyl methacrylate chloride) (also known as poly(ethyl acrylate-co-methyl methacrylate-co-trimethylammonioethyl methacrylate chloride)). In a preferred embodiment the ammonio methacrylate copolymer is selected from poly(ethyl acrylate, methyl methacrylate, trimethylammonioethyl methacrylate chloride) 1:2:0.1 (also known as ammonio methacrylate copolymer type B, or as poly(ethyl acrylate-co-methyl methacrylate-co-trimethylammonioethyl methacrylate chloride) 1:2:0.1, or as poly[ethyl propenoate-co-methyl 2-methylprop-2-enoate-co-N,N,N-trimethyl-2-[(2-methylprop-2-enoyl)oxy]ethan-1-aminium chloride]1:2:0.1, or as Eudragit® RS, e.g. commercially available as Eudragit® RS 30D); poly(ethyl acrylate, methyl methacrylate, trimethylammonioethyl methacrylate chloride) 1:2:0.2 (also known as ammonio methacrylate copolymer type A, or as poly(ethyl acrylate-co-methyl methacrylate-co-trimethylammonioethyl methacrylate chloride) 1:2:0.2, or as poly[ethyl propenoate-co-methyl 2-methylprop-2-enoate-co-N,N,N-trimethyl-2-[(2-methylprop-2-enoyl)oxy]ethan-1-aminium chloride]1:2:0.2, or as Eudragit® RL, e.g. commercially available as Eudragit® RL 30D); or a combination thereof.

According to a preferred embodiment, the solid oral dosage form of the present invention comprises apart from ammonio methacrylate copolymer, no other sustained release polymer. According to another preferred embodiment the solid oral dosage form of the present invention comprises poly(ethyl acrylate, methyl methacrylate, trimethylammonioethyl methacrylate chloride) 1:2:0.1; poly(ethyl acrylate, methyl methacrylate, trimethylammonioethyl methacrylate chloride) 1:2:0.2; or a combination thereof, and preferably no other sustained release polymer. According another preferred embodiment of the present invention, the sustained release layer (3) comprises, apart from poly(ethyl acrylate, methyl methacrylate, trimethylammonioethyl methacrylate chloride) 1:2:0.1; poly(ethyl acrylate, methyl methacrylate, trimethylammonioethyl methacrylate chloride) 1:2:0.2 (e.g. Eudragit® RL 30D); or a combination thereof, no other sustained release polymer (preferably no other polymer).

According to a particularly preferred embodiment of the present invention, the sustained release layer (3) comprises poly(ethyl acrylate, methyl methacrylate, trimethylammonioethyl methacrylate chloride) 1:2:0.1 and preferably no other sustained release polymers. According to another particularly preferred embodiment of the present invention, the sustained release layer (3) comprises poly(ethyl acrylate, methyl methacrylate, trimethylammonioethyl methacrylate chloride) 1:2:0.1 and preferably no other polymers.

The plasticizer in the sustained release layer (3) is not particularly limited. The plasticizer in the sustained release layer (3) may be selected for example from triethyl citrate (TEC), polyethylene glycol, acetyl triethyl citrate, butyl citrate, polysorbate, polypropylene glycol, dibutyl sebacate (DBS), and combinations thereof. Preferably, the plasticizer in the sustained release layer (3) of the oral dosage form of the present invention is triethyl citrate (TEC).

The anti-tacking agent in the sustained release layer (3) is not particularly limited. The anti-tacking agent in the sustained release layer (3) may be selected for example from mesoporous silica, colloidal silica dioxide, stearic acid, magnesium stearate glycerol monostearate (GMS), and talc. Preferably, the anti-tacking agent in the sustained release layer (3) of the oral dosage form of the present invention is mesoporous silica Preferably the mesoporous silica has an average particle size of 1-10 μm, preferably of 2-5 μm, more preferably about 3-4 μm, even more preferably 3.5 μm and/or an average pore volume of 1.50-1.70 cm³/g, preferably 1.55-1.65 cm³/g, more preferably 1.58-1.62 cm³/g, even more preferably about 1.60 cm³/g.

The amount of ammonio methacrylate copolymer in the sustained release layer (3) is not particularly limited. According to one embodiment the solid oral dosage form of the present invention comprises 0.2-25 wt.-%, preferably 0.5-15 wt.-%, more preferably 1-10 wt.-%, even more preferably 3-5 wt.-%, even more preferably 3.5-4.5 wt.-%, even more preferably 3.8-4.1 wt.-%, e.g. 4 wt.-%, ammonio methacrylate copolymer (preferably poly(ethyl acrylate, methyl methacrylate, trimethylammonioethyl methacrylate chloride) 1:2:0.1). According to another embodiment of the present invention the sustained release layer (3) comprises 60-90 wt.-%, preferably 65-85 wt.-%, more preferably 70-80 wt.-%, even more preferably 72-78 wt.-%, ammonio methacrylate copolymer (preferably poly(ethyl acrylate, methyl methacrylate, trimethylammonioethyl methacrylate chloride) 1:2:0.1), relative to the total weight of the solids (i.e. not including solvents, preferably water) in the sustained release layer (3).

The amount of plasticizer in the sustained release layer (3) is not particularly limited According to one embodiment of the present invention, the sustained release layer (3) comprises 2-40 wt.-%, preferably 5-30 wt.-%, more preferably 15-25 wt.-%, even more preferably 17-23 wt.-%, even more preferably 20 wt.-%, plasticizer, relative to the total weight of the ammonio methacrylate copolymers in the sustained release layer (3). According to another embodiment of the present invention the sustained release coating comprises 2-30 wt.-%, preferably 5-25 wt.-%, more preferably 10-20 wt.-%, even more preferably 12-18 wt.-%, even more preferably 13-17 wt.-%, even more preferably 14-16 wt.-%, or 15-16 wt.-% plasticizer, relative to the total weight of the solids (i.e. not including solvents, preferably water) in the sustained release layer (3).

The amount of anti-tacking agent in the sustained release layer (3) is not particularly limited. According to one embodiment of the present invention, the sustained release layer (3) comprises 1-30, wt.-%, preferably 2-25 wt.-%, more preferably 5-15 wt.-%, even more preferably 8-12 wt.-%, even more preferably 10 wt.-%, anti-tacking agent, relative to the total weight of the ammonio methacrylate copolymers in the sustained release layer (3). According to another embodiment of the present invention, the sustained release coating comprises 2-15 wt.-%, preferably 5-12 wt.-%, more preferably 6-9 wt.-%, even more preferably 6-9 wt.-%, even preferably 6.5-8.5 wt.-%, even more preferably 7-8 wt.-%, even more preferably about 7.5 wt.-% anti-tacking agent, relative to the total weight of the solids (i.e. not including solvents, preferably water) in the sustained release layer (3).

The sustained release layer (3) may or may not comprise a coalescence enhancer. According to a preferred embodiment of the present invention, the sustained release layer (3) comprises no coalescence enhancer. According to another embodiment of the present invention, the sustained release layer (3) comprises a coalescence enhancer, e.g. propylene glycol monolaurate (e.g. Lauroglycol™ 90).

According to a preferred embodiment, the sustained release layer (3) consists of ammonio methacrylate copolymer (preferably poly(ethyl acrylate, methyl methacrylate, trimethylammonioethyl methacrylate chloride) 1:2:0.1), plasticizer (preferably TEC) and anti-tacking agent (preferably mesoporous silica), and optionally residual solvent (preferably water).

According to a specific embodiment, the weight of the sustained release layer (3), relative to combined weight of the inert core unit (1) and the depot layer (2), is 4-8 wt.-%, preferably 5-7 wt.-% more preferably about 6 wt.-%.

The sustained release layer (3) may be applied to the inert core unit (1) covered (coated) with the depot layer (2) using layering. According to a preferred embodiment, the sustained release layer (3) of the solid oral dosage form of the present invention can be deposited on the depot layer (2) by layering, preferably using spray coating, more preferably fluidized-bed spray coating. Method for layering of a sustained layer onto a depot layer containing an antibody or functional fragment as an active ingredient and covering an inert core are known in the art. Such a method can be found for example in WO 2019/057562. According to a specific embodiment, the weight gain of the solid oral dosage form after sustained release coating by drug layering and subsequent drying, but before applying any further layer, is 4-8 wt.-%, preferably 5-7 wt.-%, more preferably about 6 wt.-%.

Finally, the solid oral dosage form of the present invention comprises a delayed release layer (4) covering the sustained release layer (3) and comprising poly(methacrylic acid, methyl methacrylate) 1:2, poly(methyl acrylate, methyl methacrylate, methacrylic acid) 7:3:1, poly(methacrylic acid, methyl methacrylate) 1:1, or a combination thereof, preferably poly(methacrylic acid, methyl methacrylate) 1:2; an anti-tacking agent, preferably glycerol monostearate (GMS); a plasticizer, preferably triethyl citrate (TEC), and preferably a surfactant.

The term “delayed release” is known in the art. A delayed release layer within the meaning of the present invention is a layer/coating that prevents the release of the antibody or functional fragment thereof from the solid oral dosage form, until a specific event, preferably in the form of a pH trigger in solution, occurs. For example, a delayed release layer can be designed to focus the delivery of the antibody or functional fragment thereof entirely in the large intestine, beginning at the cecum, and continuing through the ascending, transverse, and descending colon, and ending in the sigmoid colon. Alternatively, for example, a delayed release coating can be designed to begin the delivery of the antibody or functional fragment thereof in the ileum and end the release in the sigmoid colon.

The term “cover” or “covering” as used in this context is to be understood as defined above. Preferably, the delayed release layer (4) is positioned on top of the sustained release layer (3) being covered, such that there is no other layer in between (i.e. between the delayed release layer and the delayed release layer). Preferably, the delayed release layer of the solid oral dosage form of the present invention continuously covers the sustained release layer, such that there are no gaps in the delayed release layer.

The solid oral dosage form of the present invention is for oral administration, and comprises as an outer layer (is coated/coated with) a delayed release layer that prevents the release of the composition preferably before the terminal ileum, or before the ileocolonic region, alternatively before the ascending colon or before the transverse colon, of the gastrointestinal tract. The ileocolonic region is the region of the gastrointestinal tract where the small intestine merges with the large intestine. The large intestine is the penultimate section of the gastrointestinal tract and can be further subdivided into cecum, colon and rectum. The colon is further subdivided into ascending, transverse, descending colon and the sigmoid colon. The terminal ileum is the penultimate section of the small intestine and is directly adjacent to the cecum.

The approach for applying the delayed release layer is not particularly limited as long as it does not affect the stability and activity of the at least one antibody or functional fragment thereof in the depot layer coating. Methods for applying delayed release coatings are known in the art. In one embodiment of the present invention the delayed release coating is applied by spray coating, preferably fluidized-bed spray coating, e.g as described above.

The delayed release polymer in the delayed release layer (4) is selected from poly(methacrylic acid, methyl methacrylate) 1:2, poly(methyl acrylate, methyl methacrylate, methacrylic acid) 7:3:1, poly(methacrylic acid, methyl methacrylate) 1:1, or a combination thereof. Poly(methacrylic acid, methyl methacrylate) 1:2 is also known as methacrylic acid-methyl methacrylate copolymer (1:2), or as poly(methacrylic acid-co-methyl methacrylate) 1:2, or as Eudragit® S. Poly(methyl acrylate, methyl methacrylate, methacrylic acid) 7:3:1 is also known as poly(methyl acrylate-co-methyl methacrylate-co-methacrylic acid) 7:3:1, or as Eudragit® FS. Poly(methacrylic acid, methyl methacrylate) 1:1 is also known as methacrylic acid-methyl methacrylate copolymer (1:1), or as poly(methacrylic acid-co-methyl methacrylate) 1:1, or as Eudragit® L.

According to one embodiment the delayed release layer (4) comprises no polymers that are not enteric polymers. The term “enteric polymer” is known in the art and may refer to a polymer that does not dissolve in the gastric environment. According to another embodiment of the present invention the delayed release layer (4) comprises, apart from poly(methacrylic acid, methyl methacrylate) 1:2, poly(methyl acrylate, methyl methacrylate, methacrylic acid) 7:3:1, poly(methacrylic acid, methyl methacrylate) 1:1, or a combination thereof, no other delayed release polymers. According to a preferred embodiment of the present invention, the delayed release layer (4) comprises, poly(methacrylic acid, methyl methacrylate) 1:2, and preferably no other delayed release polymer.

According to one embodiment of the present invention, the solid oral dosage form comprises 5-40 wt.-%, preferably 10-40 wt.-%, more preferably 10-35 wt.-%, even more preferably 15-35 wt.-%, even more preferably 15-30 wt.-%, even more preferably 18-30 wt.-%, even more preferably 20-30 wt.-%, even more preferably 21-26 wt.-%, e.g. about 22-25 wt.-%, poly(methacrylic acid, methyl methacrylate) 1:2, poly(methyl acrylate, methyl methacrylate, methacrylic acid) 7:3:1, poly(methacrylic acid, methyl methacrylate) 1:1, or a combination thereof (preferably poly(methacrylic acid, methyl methacrylate) 1:2). According to another embodiment of the present invention, the delayed release layer (4) comprises 50-97 wt.-%, preferably 65-95 wt.-%, more preferably 70-90 wt.-%, even more preferably 75-85 wt.-%, e.g. about 80 wt.-%, poly(methacrylic acid, methyl methacrylate) 1:2, poly(methyl acrylate, methyl methacrylate, methacrylic acid) 7:3:1, poly(methacrylic acid, methyl methacrylate) 1:1, or a combination thereof (preferably poly(methacrylic acid, methyl methacrylate) 1:2), relative to the total weight of the solids (i.e. not including residual solvent, e.g. ethanol and/or water) in the delayed release layer (4).

According to another embodiment of the present invention, the solid dosage form (in the delayed release layer) comprises 7-12 mg/cm², preferably 8-11 mg/cm², more preferably 9-10 mg/cm², even more preferably about 9.5 mg/cm², poly(methacrylic acid, methyl methacrylate) 1:2, poly(methyl acrylate, methyl methacrylate, methacrylic acid) 7:3:1, poly(methacrylic acid, methyl methacrylate) 1:1, or a combination thereof (preferably poly(methacrylic acid, methyl methacrylate) 1:2).

The plasticizer in the delayed release layer (4) is not particularly limited. The plasticizer in the delayed release layer (4) may be selected for example from triethyl citrate (TEC), polyethylene glycol, acetyl triethyl citrate, butyl citrate, polysorbate, polypropylene glycol, dibutyl sebacate (DBS), or a combination thereof. Preferably the plasticizer in the delayed release layer (4) is triethyl citrate (TEC). According to one embodiment of the present invention, the delayed release layer (4) comprises 1-30 wt.-%, preferably 2-25 wt.-%, more preferably 5-15 wt.-%, even more preferably 8-12 wt.-%, even more preferably about 10 wt.-%, plasticizer, relative to the total weight of the delayed release polymers in the delayed release layer (4). According to another embodiment of the present invention, the delayed release layer (4) comprises 1-25 wt.-%, preferably 4-15 wt.-%, more preferably 5-12 wt.-%, even more preferably 6-10 wt.-%, even more preferably 7-9 wt.-%, even more preferably about 8 wt.-%, plasticizer, relative to the total weight of the solids (i.e. not including residual solvents, e.g. ethanol and/or water) in the delayed release layer (4).

The anti-tacking agent in the delayed release layer (4) is not particularly limited. The anti-tacking agent in the delayed release layer (4) may be selected for example from mesoporous silica, colloidal silica dioxide, stearic acid, magnesium stearate, glycerol monostearate (GMS), or talc. Preferably the anti-tacking agent in the delayed release layer (4) is glycerol monostearate (GMS). According to one embodiment of the present invention, the delayed release layer (4) comprises 1-30 wt.-%, preferably 2-25 wt.-%, more preferably 5-15 wt.-%, even more preferably 8-12 wt.-%, even more preferably about 10 wt.-%, anti-tacking agent, relative to the total weight of the delayed release polymers in the delayed release layer (4). According to another embodiment of the present invention, the delayed release layer (4) comprises 1-20 wt.-%, preferably 4-15 wt.-%, more preferably 5-12 wt.-%, even more preferably 6-10 wt.-%, even more preferably 7-9 wt.-%, even more preferably about 8 wt.-%, anti-tacking agent, relative to the total weight of the solids (i.e. not including residual solvents, e.g. ethanol and/or water) in the delayed release layer (4).

The delayed release layer (4) of the solid oral dosage form of the present invention optionally may further comprise a surfactant. The surfactant in the delayed release layer (4) is not particularly limited, however preferably it is a nonionic surfactant. The solid oral dosage form in the delayed release layer (4) may comprise a surfactant selected from the group consisting of polysorbate 20, polysorbate 28, polysorbate 40, polysorbate 60, polysorbate 65, polysorbate 80, polysorbate 81, polysorbate 85, poloxamer 124, poloxamer 181, poloxamer 188, poloxamer 237, poloxamer 331, poloxamer 338 and poloxamer 407, and combinations thereof.

According to a preferred embodiment of the present invention, the surfactant in the delayed release layer (4) is polysorbate 20, 28, 40, 60, 65, 80, 81 and 85; preferably polysorbate 80. According to one embodiment of the present invention, the delayed release layer (4) comprises 0.1-15 wt.-%, preferably 0.5-10 wt.-%, more preferably 1-6 wt.-%, even more preferably 2-5 wt.-%, even more preferably 3-4.5 wt.-%, even more preferably 3.5-4 wt.-%, even more preferably about 4%, surfactant, relative to the total weight of the delayed release polymers in the delayed release layer (4). According to another embodiment of the present invention, the delayed release layer (4) comprises 0.1-15 wt.-%, preferably 0.5-10 wt.-%, more preferably 1-5 wt.-%, even more preferably 2-4 wt.-%, even more preferably 2.5-3.5 wt.-%, even more preferably 3-3.5 wt.-%, even more preferably about 3-3.2 wt.-%, surfactant, relative to the total weight of the solids (i.e. not including solvent) in the delayed release layer (4).

According to a preferred embodiment of the solid oral dosage form of the present invention, the delayed release layer (4) consists of poly(methacrylic acid, methyl methacrylate) 1:2, poly(methyl acrylate, methyl methacrylate, methacrylic acid) 7:3:1, poly(methacrylic acid, methyl methacrylate) 1:1, or a combination thereof (preferably poly(methacrylic acid, methyl methacrylate) 1:2); anti-tacking agent (preferably glycerol monostearate (GMS)); plasticizer (preferably triethyl citrate (TEC)); surfactant (preferably polysorbate 80), and optionally residual solvent (e.g. ethanol and/or water).

In one embodiment of the present invention the residual solvent content of the solid oral dosage form (e.g. residual solvent, preferably water, content after preparation of the solid oral dosage form), relative to the total weight of the solid oral dosage form, is less than 15 wt.-%, preferably less than 10 wt.-%, more preferably less than 8 wt.-%, even more preferably less than 7%, even more preferably less than 6%, even more preferably no more than 5%, e.g. about 5%, or alternatively no more than about 4 wt.-% or no more than about 3 wt.-%. In another embodiment of the present invention the residual water content of the solid oral dosage form, relative to the total weight of the solid oral dosage form, is less than 15 wt.-%, preferably less than 10 wt.-%, more preferably less than 8 wt.-%, even more preferably less than 7%, even more preferably no more than 5%, e.g. about 5%, or alternatively no more than about 4 wt.-%, or no more than about 3 wt.-%, or no more than about 2 wt.-%.

In a preferred embodiment of the present invention the solid oral dosage form comprises, or consists of, a) in the inert core unit, microcrystalline cellulose; and/or b) in the depot layer, antibody or functional fragment thereof e.g. specific to TNFα, sucrose, L-histidine preferably L-histidine monohydrochloride and/or free base, hypromellose (HPMC), preferably hypromellose 2910 2.6-3.6 mPas, mesoporous silica, and/or polysorbate 80; and/or c) in the sustained release layer, poly(ethyl acrylate, methyl methacrylate, trimethylammonioethyl methacrylate chloride) 1:2:0.1, triethyl citrate (TEC) and/or mesoporous silica; and/or d) in the delayed release layer, poly(methacrylic acid, methyl methacrylate) 1:2, TEC, polysorbate 80, and/or glycerol monostearate (GMS); and/or e) residual water in the inert core unit and/or any of the layers.

In a preferred embodiment of the solid oral dosage form of the present invention, the inert core unit comprises 5-75 wt.-%, preferably 15-65 wt.-%, more preferably 15-60 wt.-%, even more preferably 20-60 wt.-%, even more preferably 25-30 wt.-%, microcrystalline cellulose, relative to the total weight of the solid oral dosage form.

In another preferred embodiment of the solid oral dosage form of the present invention, the depot layer (2) comprises, relative to the total weight of the solid oral dosage form, 0.05-30 wt.-% antibody or functional fragment thereof, 0.03-30 wt.-% binder, 0.1-40 wt.-% sucrose, 0.005-5 wt.-% L-histidine (salt and/or free base) and optionally 0.001-5 wt.-% other buffers, 0.005-8 wt.-% anti-tacking agent, and/or 0.0005-2 wt.-% surfactant; preferably 0.1-20 wt.-% antibody or functional fragment thereof, 0.1-25 wt.-% binder, 0.5-35 wt.-% sucrose, 0.01-3 wt.-% L-histidine and optionally 0.01-3 wt.-% other buffers, 0.01-5 wt.-% anti-tacking agent, and/or 0.001-1 wt.-% surfactant; more preferably 0.5-20 wt.-% antibody or functional fragment thereof, 0.5-15 wt.-% binder, 1-25 wt.-% sucrose, 0.05-2 wt.-% L-histidine and optionally 0.05-1.5 wt.-% other buffers, 0.05-1.5 wt.-% anti-tacking agent, and/or 0.005-0.3 wt.-% surfactant; even more preferably 2-20 wt.-% antibody or functional fragment thereof, 5-15 wt.-% binder, 10-25 wt.-% sucrose, 0.1-2 wt.-% L-histidine and optionally 0.5-1.5 wt.-% other buffers, 0.5-1.5 wt.-% anti-tacking agent, and/or 0.05-0.3 wt.-% surfactant; even more preferably 8-12 wt.-% antibody or functional fragment thereof, 6-8 wt.-% binder, 15-18 wt.-% sucrose, 0.15-1 wt.-% L-histidine and optionally 0.6-0.9 wt.-% other buffers, 0.6-0.8 wt.-% anti-tacking agent (preferably mesoporous silica), and/or 0.05-0.1 wt.-% surfactant (preferably polysorbate 80).

In another preferred embodiment of the solid oral dosage form of the present invention, the sustained release layer (3) comprises, relative to the total weight of the solid oral dosage form, 1-10 wt.-% ammonio methacrylate copolymer, 0.1-5 wt.-% plasticizer, and/or 0.05-7 wt.-% anti-tacking agent; preferably 3-6 wt.-% ammonio methacrylate copolymer, 0.2-2 wt.-% plasticizer, and/or 0.1-3 wt.-% anti-tacking agent; more preferably 3.3-4.5 wt.-% ammonio methacrylate copolymer, 0.5-1.5 wt.-% plasticizer, and/or 0.2-1 wt.-% anti-tacking agent; even more preferably 3.6-4.2 wt.-% poly(ethyl acrylate, methyl methacrylate, trimethylammonioethyl methacrylate chloride) 1:2:0.1 (e.g. Eudragit® RS 30D), 0.6-1 wt.-% plasticizer (preferably triethyl citrate (TEC)) and/or 0.3-0.5 wt.-% anti-tacking agent.

In another preferred embodiment of the solid oral dosage form of the present invention, the delayed release layer (4) comprises, relative to the total weight of the solid oral dosage form, 10-40 wt.-% poly(methacrylic acid, methyl methacrylate) 1:2, 0.5-10 wt.-% plasticizer, 0.1-3.5 wt.-% surfactant, and/or 0.5-10 wt.-% anti-tacking agent (preferably glycerol monostearate (GMS)); preferably 15-35 wt.-% poly(methacrylic acid, methyl methacrylate) 1:2, 1-5 wt.-% plasticizer, 0.2-2 wt.-% surfactant, and/or 1-5 wt.-% anti-tacking agent (preferably glycerol monostearate (GMS)); more preferably 18-30 wt.-% poly(methacrylic acid, methyl methacrylate) 1:2, 1.5-3 wt.-% plasticizer, 0.5-1.3 wt.-% surfactant, and/or 1.5-3 wt.-% anti-tacking agent (preferably glycerol monostearate (GMS)); even more preferably 21-26 wt.-% poly(methacrylic acid, methyl methacrylate) 1:2, 2.0-2.6 wt.-% plasticizer, 0.7-1.1 wt.-% surfactant, and/or 2.0-2.6 wt.-% anti-tacking agent (preferably glycerol monostearate (GMS)).

In another preferred embodiment of the present invention, the solid oral dosage form comprises up to 8 wt.-%, up to 7 wt.-%, or up to 6 wt.-%, or up to 5.5 wt.-%, preferably up to 5 wt.-%, e.g. about 5, or 4.5, or 4, or 3 wt.-%, water in the inert core unit and/or any of the layers, relative to the total weight of the solid oral dosage form.

In yet another preferred embodiment of the present invention the solid oral dosage form, relative to the total weight of the solid oral dosage form, comprises, or consists of, a) in the inert core unit, 5-75 wt.-%, preferably 15-65 wt.-%, more preferably 20-60 wt.-%, even more preferably 25-30 wt.-%, microcrystalline cellulose; and/or b) in the depot layer, 0.05-30 wt.-% antibody or functional fragment thereof, 0.03-30 wt.-% binder, 0.1-40 wt.-% sucrose, 0.005-5 wt.-% L-histidine (salt and/or free base) and optionally 0.001-5 wt.-% other buffers, 0.005-10 wt.-% anti-tacking agent, and/or 0.0005-2 wt.-% surfactant; preferably 0.1-20 wt.-% antibody or functional fragment thereof, 0.1-25 wt.-% binder, 0.5-35 wt.-% sucrose, 0.01-3 wt.-% L-histidine and optionally 0.01-3 wt.-% other buffers, 0.01-5 wt.-% anti-tacking agent, and/or 0.001-1 wt.-% surfactant; more preferably 0.5-20 wt.-% antibody or functional fragment thereof, 0.5-15 wt.-% binder, 1-25 wt.-% sucrose, 0.05-2 wt.-% L-histidine and optionally 0.05-1.5 wt.-% other buffers, 0.05-1.5 wt.-% anti-tacking agent, and/or 0.005-0.3 wt.-% surfactant; even more preferably 2-20 wt.-% antibody or functional fragment thereof, 5-15 wt.-% binder, 10-25 wt.-% sucrose, 0.1-2 wt.-% L-histidine and optionally 0.5-1.5 wt.-% other buffers, 0.5-1.5 wt.-% anti-tacking agent, and/or 0.05-0.3 wt.-% surfactant; even more preferably 8-12 wt.-% antibody or functional fragment thereof, 6-8 wt.-% binder, 15-18 wt.-% sucrose, 0.15-1 wt.-% L-histidine and optionally 0.6-0.9 wt.-% other buffers, 0.6-0.8 wt.-% mesoporous silica, and/or 0.05-0.1 wt.-% polysorbate 80; and/or c) in the sustained release layer, 1-10 wt.-% ammonio methacrylate copolymer, 0.1-5 wt.-% plasticizer, and/or 0.05-7 wt.-% anti-tacking agent; preferably 3-6 wt.-% ammonio methacrylate copolymer, 0.2-2 wt.-% plasticizer and/or 0.1-3 wt.-% anti-tacking agent; more preferably 3.3-4.5 wt.-% ammonio methacrylate copolymer, 0.5-1.5 wt.-% plasticizer, and/or 0.2-1 wt.-% anti-tacking agent; preferably 3.6-4.2 wt.-% poly(ethyl acrylate, methyl methacrylate, trimethylammonioethyl methacrylate chloride) 1:2:0.1, 0.6-1 wt.-% triethyl citrate (TEC) and/or 0.3-0.5 wt.-% mesoporous silica; and/or d) in the delayed release layer, 10-40 wt.-% poly(methacrylic acid, methyl methacrylate) 1:2, 0.5-5 wt.-% plasticizer, 0.1-3.5 wt.-% surfactant, and/or 0.5-8 wt.-% glycerol monostearate (GMS); preferably 15-35 wt.-% poly(methacrylic acid, methyl methacrylate) 1:2, 1-4 wt.-% plasticizer, 0.2-2 wt.-% surfactant, and/or 1-5 wt.-% glycerol monostearate (GMS); more preferably 18-30 wt.-% poly(methacrylic acid, methyl methacrylate) 1:2, 1.5-3 wt.-% plasticizer, 0.5-1.3 wt.-% surfactant, and/or 1.5-3 wt.-% glycerol monostearate (GMS); even more preferably 21-26 wt.-% poly(methacrylic acid, methyl methacrylate) 1:2, 2.0-2.6 wt.-% plasticizer, 0.7-1.1 wt.-% surfactant (e.g. polysorbate 80), and/or 2.0-2.6 wt.-% glycerol monostearate (GMS); and/or e) up to 5.5 wt.-%, or up to 5 wt.-%, e.g. 5 wt.-%, water in the inert core unit and/or any of the layers.

The activity and stability of the antibody or functional fragment thereof, e.g. the anti-TNFα antibody or functional fragment thereof, in the solid oral dosage form of the present invention is high. The stability and activity of an antibody or fragment thereof can be estimated for example by determining the fraction of an antibody or functional fragment thereof present as dimers and other aggregates. According to one embodiment of the present invention, the fraction of total content of antibody or functional fragment thereof present in the solid oral dosage form as dimers and other aggregates does not exceed more than 15%, preferably 12%, more preferably 10%, even more preferably 8%, even more preferably 7%, even more preferably 5%, e.g. 3%. Methods to determine the fraction of a polypeptide present as dimers and other aggregates are known in the art, and include for example Size Exclusion Chromatography (SEC). According to another embodiment the fraction of total content of antibody or functional fragment thereof present in the solid oral dosage form as fragments of the full-length antibody or functional fragment thereof does not exceed 15%, preferably 12%, more preferably 10%, even more preferably 8%, even more preferably 7%, even more preferably 5%, e.g. 3%.

The solid oral dosage form of the present invention preferably is gastric resistant. According to one embodiment of the present invention, the solid oral dosage form of the present invention is for targeted release of the antibody or functional fragment thereof starting in the terminal ileum, the ileocolonic region, the ascending colon or transverse colon, preferably in the terminal ileum, the ileocolonic region or the ascending colon, more preferably in the terminal ileum or the ileocolonic region. According to another embodiment of the present invention, the solid oral dosage form of the present invention is for targeted release of the antibody or functional fragment thereof in the terminal ileum, the ileocolonic region, the ascending colon, the transverse colon, descending colon and/or the sigmoid colon. According to yet another embodiment of the present invention, the solid oral dosage form of the present invention is for targeted sustained release of the antibody or functional fragment thereof starting in the terminal ileum and continuing at least through the descending colon, preferably the sigmoid colon.

The solid oral dosage form of the present invention is suitable for targeted sustained release in the lower GI tract. Preferably, the solid oral dosage form of the present invention, upon immersion in an aqueous solution at a pH 6.0 under continuous agitation (e.g. for 2 h), does not release any relevant amount of antibody or functional fragment thereof (e.g. releases less than 1 wt.-%, or less than 0.5 wt.-%, or less than 0.1 wt.-% of the total antibody or functional fragment thereof load). According to one embodiment of the invention, the solid oral dosage form according to any of the embodiments described above allows a sustained release of the antibody or functional fragment thereof over a time period of at least 5 h, preferably at least 10 h, more preferably at least 12 h, even more preferably at least 15 h, even more preferably at least 20 h, most preferably at least 24 h, upon continuously immersing the solid oral dosage form in an aqueous solution under continuous agitation at a pH of 6.5-7.5, preferably a pH of about 6.8. According to another embodiment of the invention, upon continuously immersing the solid oral dosage form according to any of the embodiments described above in an aqueous solution under continuous agitation and at a pH of 6.5-7.5, preferably pH of 6.5-7.0, more preferably a pH of 6.8, a sustained release, preferably with a substantially constant release rate, of at least 80%, preferably at least 90%, 95%, or 98%, of the antibody or fragment thereof in the solid oral dosage form over 4-30 h, preferably 8-28 h, more preferably 16-26 h, even more preferably 24 h, is achieved.

In a further aspect the present invention is directed to the solid oral dosage form as defined in any of the embodiments above for use in the treatment of a disease in the gastrointestinal tract, preferably in the ileum (preferably the terminal ileum), the ileocolonic region, the ascending colon, transverse colon, the descending colon and/or the sigmoid colon of a patient, preferably a human patient. Such diseases include e.g. immune checkpoint inhibitor (ICPI) induced colitis, ICPI induced enterocolitis, ICPI induced diarrhoea, IBD, cancer (such as colorectal cancer or small intestine cancer), celiac disease, infections (such as Clostridium difficile infection) of the small intestine and the colon, and diarrhea. In a preferred embodiment of the present invention, the solid oral dosage form of the present invention is for use in the treatment of ICPI induced colitis, ICPI induced enterocolitis, or ICPI induced diarrhoea, preferably ICPI induced colitis. In another preferred embodiment of the present invention, the solid oral dosage form of the present invention is for use in the treatment of an IBD, e.g. Crohn's disease or ulcerative colitis.

In another aspect, the present invention is directed to an oral multiparticulate drug delivery system, comprising a plurality of solid oral dosage forms of any of the embodiments described above, preferably wherein the oral multiparticulate drug delivery system is a sachet/stick pack, a straw device (xStraw®), capsule, or tablet/mini-tablet, more preferably a capsule; preferably wherein the oral multiparticulate drug delivery system comprises a total amount of the antibody or functional fragment thereof suitable for oral administration to a human patient.

According to a further embodiment, the oral multiparticulate drug delivery system of the invention comprises a plurality of solid oral dosage forms of any of the embodiments described above, wherein each solid oral dosage form preferably has a predetermined axis and a predetermined cross-sectional profile, wherein at least 80% by number of those solid oral dosage forms, preferably 90%, more preferably 95%, have a median aspect ratio between 0.7 and 1.7, the aspect ratio being defined as solid oral dosage form length along the predetermined axis divided by the smallest cross-sectional dimension. According to another embodiment the median aspect ratio is above 0.8, preferably above 0.9, and below 1.6, preferably below 1.5, more preferably 1.4, even more preferably below 1.3, even more preferably below 1.2, most preferably about 1. For further details regarding aspect ratio, predetermined axis, predetermined cross-sectional profile and span (including definitions and embodiments), it is referred to the disclosure of EP 2 512 453. It is to be understood that the above definitions and embodiments regarding aspect ratio and span of the solid oral dosage form equally apply to the inventive solid oral dosage forms according to any one of the embodiments above. Further embodiments for the oral multiparticulate drug delivery system may be found in EP 2 512 453 and are applicable to the present invention independent of whether these embodiments are disclosed in EP 2 512 453 as referring to spheronized/non-spheronized solid dosage forms.

According to yet another embodiment of the present invention, the oral multiparticulate drug delivery system of the invention allows the recovery of at least 60%, preferably at least 80%, more preferably at least 85%, even more preferably at least 93%, even more preferably at least 95%, even more preferably at least 97%, even more preferably at least 98%, of the antibody or functional fragment thereof from the solid oral dosage forms.

According to yet another embodiment of the present invention, the oral multiparticulate drug delivery system of the invention allows the recovery of at least 60%, preferably at least 80%, more preferably at least 85%, even more preferably at least 93%, even more preferably at least 95%, even more preferably at least 97%, even more preferably at least 98%, of the antibody or functional fragment thereof from the solid oral dosage forms, within 4 h, or 6 h, or 8 h, or 10 h, or 12 h, or 14 h, or 16 h, or 18 h, or 20 h, or 22 h, or 24 h, or 26 h, or 28 h, or 30 h, of continuously immersing the solid oral dosage form in an aqueous solution under continuous agitation (sustained release), at a pH of 6.5 to 7, preferably at a pH of about 6.8.

According to yet another embodiment of the present invention, the oral multiparticulate drug delivery system of the invention is prepared from a plurality of solid oral dosage forms by compression or encapsulation, preferably encapsulation.

In a further aspect, the present invention is directed to the use of the solid oral dosage form as described in any of the embodiments above in the preparation of a medicament for the treatment of a gastrointestinal disease. The medicament may comprise the oral multiparticulate drug delivery system as described in any of the embodiments above. According to one embodiment the gastrointestinal disease is immune checkpoint inhibitor (ICPI) induced colitis, ICPI induced enterocolitis, ICPI induced diarrhoea, or IBD (e.g. Crohn's disease or ulcerative colitis).

In yet a further aspect, the present invention is directed to a method for targeted local treatment of a gastrointestinal disease, comprising administering to a patient in need thereof a pharmaceutically effective amount of the solid oral dosage form of any of the embodiments described above or the oral multiparticulate drug delivery system of any of the embodiments described above. The patient preferably is a human patient. According to one embodiment, the method is for the treatment of the GI disease in the ileum (preferably the terminal ileum), the ileocolonic region, the ascending colon, transverse colon, the descending colon and/or the sigmoid colon of the patient. According to another embodiment of the method, the gastrointestinal disease is an inflammatory bowel disease (IBD), e.g. Crohn's disease or ulcerative colitis. According to a preferred embodiment of the method, the gastrointestinal disease is immune checkpoint inhibitor (ICPI) induced colitis, ICPI induced enterocolitis, or ICPI induced diarrhoea, preferably ICPI induced colitis.

TABLE 1
Overview of the sequences of the sequence listing.

| SEQ | |
|---|---|
| ID | |
| NO: | Description of the amino acid sequence |
| 1 | CDR L1 of clone 16-22-H05 |
| 2 | CDR L2 of clone 16-22-H05 |
| 3 | CDR L3 of clone 16-22-H05 |
| 4 | CDR H1 of clone 16-22-H05 |
| 5 | CDR H2 of clone 16-22-H05 |
| 6 | CDR H3 of clone 16-22-H05 |
| 7 | V_{H} of humanized IgG of clone 16-22-H05 |
| 8 | V_{L} of humanized IgG of clone 16-22-H05 |
| 9 | CDR L1 of clone 17-22-B03 |
| 10 | CDR L2 of clone 17-22-B03 |
| 11 | CDR L3 of clone 17-22-B03 |
| 12 | CDR H1 of clone 17-22-B03 |
| 13 | CDR H2 of clone 17-22-B03 |
| 14 | CDR H3 of clone 17-22-B03 |
| 15 | V_{H} of humanized IgG of clone 17-22-B03 |
| 16 | V_{L} of humanized IgG of clone 17-22-B03 (sc08) |
| 17 | V_{L} of humanized IgG of clone 17-22-B03 (sc02) |
| 18 | Fc region of Ab-REW (including hinge region) |
| 19 | Fc region of Ab-YTE-DLE-PVAdeltaG (including hinge region) |
| 20 | Fc region of Ab-A-DLE-PVAdeltaG (including hinge region) |
| 21 | Fc region of Ab-A-AEA-PVAdeltaG (including hinge region) |
| 22 | Fc region of Ab-AA (including hinge region) |
| 23 | Light chain of humanized IgG of clone 16-22-H05 |
| 24 | Light chain of humanized IgG of clone 17-22-B03 (sc08) |
| 25 | Light chain of humanized IgG of clone 17-22-B03 (sc02) |
| 26 | Heavy chain of Ab-REW (based on clone 16-22-H05) |
| 27 | Heavy chain of Ab-REW (based on clone 17-22-B03) |
| 28 | Heavy chain of Ab-YTE-DLE-PVAΔG |
| | (based on clone 16-22-H05) |
| 29 | Heavy chain of Ab-A-DLE-PVAΔG (based on clone 16-22-H05) |
| 30 | Heavy chain of Ab-A-AEA-PVAΔG (based on clone 16-22-H05) |
| 31 | Heavy chain of Ab-YTE-DLE-PVAΔG |
| | (based on clone 17-22-B03) |
| 32 | Heavy chain of Ab-A-DLE-PVAΔG (based on clone 17-22-B03) |
| 33 | Heavy chain of Ab-A-AEA-PVAΔG (based on clone 17-22-B03) |
| 34 | Heavy chain of Ab-AA (based on clone 16-22-H05) |
| 35 | Heavy chain of Ab-AA (based on clone 17-22-B03) |

### EXAMPLES

### Materials

A summary of materials used in this study is presented in Table 2.

TABLE 2
Summary of materials

| Description | Supplier |
|---|---|
| Antibody: ‘MB-001 DS’ | |
| (light chain: SEQ ID NO: 23; | |
| heavy chain: SEQ ID NO: 26) | |
| Cellets 1000 | Pharmatrans Sanaq |
| L-Histidine | Merck |
| L-Histidine HCl monohydrochloride | Merck |
| Pharmacoat 603 | Shin-Etsu |
| Syloid 244 FP | Grace |
| Polysorbate 80 | Sigma-Aldrich |
| Eudragit RL 30D | Evonik |
| Eudragit RS 30D | Evonik |
| Aquacoat ECD 30 | Colorcon |
| Triethyl citrate | Tillotts (ERP) |
| Eudragit S100 | Evonik |
| Glycerol monostearate Type II | Tillotts (ERP) |
| Ethanol 96% | Sigma Aldrich |
| Purified water | Tillotts Ziefen |
| Bradford reagent (Coomassie | Thermo Scientic |
| Plus protein assay reagent) | |
| Phosphate buffer pH 6.8 | AD |

A description of the formulations evaluated in this study is summarized in Table 3.

TABLE 3
Summary of formulations in this study

| Example No. | 1 | 2 | 3* |
|---|---|---|---|
| Core | microcrystalline cellulose (Cellets 1000) | | |
| Drug layer | Antibody | | |
| | Syloid 244 FP | | |
| | Histidine monohydrochloride monohydrate | | |
| | L-Histidine (free base) | | |
| | Sucrose | | |
| | Polysorbate 80 | | |
| | Hypromellose (Pharmacoat 603) | | |
| Sustained | Syloid 244 FP | Syloid 244 FP | Triethyl citrate |
| release layer | Triethyl citrate | Triethyl citrate | Ethylcellulose |
| | Eudragit RL | Eudragit RS | (Aquacoat |
| | | | ECD 30) |

| | |
|---|---|
| Delayed | Eudragit S |
| release layer | Triethyl citrate |
| | Glycerol monostearate (GMS) |
| | Polysorbate 80 |

*Syloid or any other anti-tacking is not recommended by the general guidelines from the suppliers of Ethylcellulose (for Eudragit RL/RS an anti-tacking is recommended). Therefore, the trials were conducted without any element that might (at least theoretically) rather compromise the quality of the ethylcellulose coating layer. The suppliers of Aquacoat ECD 30% recommend to only add the plasticizer to the ready to use Ethylcellulose dispersion.

### Example 1

Preparation of a Multilayered Formulation with a Sustained Release Layer Comprising the Cationic Polymer Eudragit RL

### 1.1 Manufacture of Drug Layered Pellets

Antibody-layered pellets were produced by spraying a dispersion containing antibody onto inert microcrystalline cellulose cellets (Cellets 1000) in a Mini-Glatt fluidized bed coater. The fluid bed was fitted with the micro kit to enable a bottom spray process (without the Wurster insert).

A 20 mMol histidine buffer (500 ml) was prepared by dispensing 400 ml of purified water into a 500 ml volumetric cylinder and adding 0.373 g L-Histidine and 1.593 g L-Histidine HCl under magnetic stirring (500 rpm) until a clear solution. The magnetic stirrer bar was removed prior to adding additional purified water up to 500 ml with continued stirring. The pH of the final buffer was measured using a calibrated pH meter (final pH was determined as 5.59).

A quantity of histidine buffer (8.63 g) was dispensed into a 25 ml glass beaker. The required quantity of Pharmacoat 603 (1.250 g) was added under magnetic stirring (500 rpm for at least 30 minutes). The required quantity of Syloid 244 FP (0.125 g) was added to the glass beaker. The dispersion was left stirring overnight.

Antibody was pre-diluted with purified water to obtain an antibody stock solution at a concentration of 45 mg/ml. Prior to the pre-dilution with water, the thawed antibody solution (already equilibrated to 25° C.) was homogenized by manually rotating the bottle for 2 minutes. Thereafter, the required quantity of MB-001 DS (36.9 g) was dispensed into a 100 ml plastic beaker. The required quantity of purified water (3.1 g) was added to the beaker under magnetic stirring (200 rpm for at least 10 minutes).

Thereafter, the corresponding amount of the HPMC coating suspension was slowly added to the antibody stock solution in the plastic beaker under magnetic stirring to achieve the final coating formulation for drug layering. The drug layer formulation was mixed for at least 30 minutes by magnetic stirring prior to starting the coating process.

An excess quantity of cellets 1000 were sieved using a 1.25 mm sieve to exclude oversized cellets and agglomerates and 0.8 mm sieve to exclude fragments. A quantity of sieved cellets (80 g) were dispensed for drug layer coating. Cellets were coated with the drug layer (antibody) formulation.

The drug layered pellets were removed from the product container at the end of coating process and distributed on a stainless-steel tray. The drug layered pellets were tray dried in a ventilated oven set at 40° C. (0.6 bar airflow) for 22 hours.

A quantity of dried drug layered pellets (70 g) was weighed into a 100 ml brown glass bottle for additional coating trials. The bottle was closed and sealed with parafilm. The remaining quantity of pellets were transferred to a small glass bottle for humidity determination. Pellets were weighed before and after layering and drying to enable calculation of the theoretical antibody loading. The theoretical loading of the antibody was calculated based on the amount of total solids layered after each step (accounting for residual moisture) and the known antibody concentration in the spraying dispersion.

### 1.2 Sustained Release Layer

Drug layered pellets were coated with an aqueous sustained release dispersion containing Eudragit RL as a functional polymer (Eudragit RL 30 D available as a 30% dispersion).

The bottle containing Eudragit RL 30D was manually shaken for 2 minutes. The Eudragit RL 30D was dispensed by pouring through a 0.5 mm mesh into a tared 50 ml glass beaker on a balance until the required quantity (15.385 g) was achieved. The required quantity of purified water (13.231 g) was added to the beaker under magnetic stirring (400 rpm for 10 minutes). The required quantity of triethyl citrate (0.923 g) was added to the beaker. Stirring was continued for an additional 10 minutes before adding the required quantity of Syloid 244 FP (0.462 g) to the beaker. Stirring was continued for an additional 30 minutes.

An anti-tacking suspension was prepared by dispensing the required quantity of purified water (4.34 g) into a 20 ml glass beaker. The required quantity of Syloid 244 FP (0.38 g) was added to the beaker under magnetic stirring (200 rpm). Stirring was continued for at least 30 minutes.

Approximately 35 g of low antibody-loaded pellets obtained in 1.1 were coated with the Eudragit RL dispersion to a achieve a 6% polymer weight gain. Pellets were coated in the Glatt Mini/Midi coater fitted with the micro kit using the bottom spray configuration.

After the coating process, the anti-tacking dispersion of Syloid 244FP in purified water was sprayed onto the pellets to reduce stickiness (6.25%, based on the target pellet weight after Eudragit RL coating).

Curing was performed in-process for 30 minutes (at a product temperature of 43.7-44.0° C.). During the curing process, 240 g of purified water per kg (starting batch size) was sprayed to create a moist environment required for curing of the polymer.

Approximately 20 g of Eudragit RL coated pellets were dispensed into a 30 ml glass bottle for further coating trials. The remaining pellets were dispensed into an additional 30 ml glass bottle for characterization (LOD, average pellet mass and surface area).

### 1.3 Eudragit S Enteric Coating

Eudragit S coating formulations were prepared in three steps.

The 1st step involved manufacturing a GMS emulsion. The required quantity of purified water (19.5 g) and polysorbate 80 (0.419 g) were dispensed into a 25 ml glass beaker under stirring (level 2). The solution was heated up to 75° C. before adding the required quantity of GMS (1.048 g) to the beaker. Stirring was continued at 75° C. for 15 minutes. The heater was switched off to allow the emulsion to cool down to room temperature with continued stirring at 200 rpm.

The 2nd step involved manufacturing a Eudragit S organic solution. A Eudragit S solution was prepared by dispensing the required quantity of ethanol 96% (97.50 g) into a 200 ml glass beaker. The required quantity of triethyl citrate (1.048 g) was added to the beaker under magnetic stirring (400 rpm for at least 5 minutes). The required quantity of Eudragit S 100 (10.484 g) was slowly added to the beaker. Stirring was continued for at least 60 minutes to achieve a clear solution.

The 3rd step involved finalizing the coating formulation. The final coating dispersion was prepared by slowly adding the GMS emulsion to the Eudragit S organic solution whilst stirring at 400 rpm. Stirring was continued for an additional 30 minutes.

Approximately 20 g of sustained release coated pellets were coated with the Eudragit S coating formulation. Pellets were coated in the Glatt Mini/Midi coater fitted with the micro kit using the bottom spray configuration.

The Eudragit S organic coating dispersion was sprayed onto the sustained release (intermediate) pellets in a fluidized bed to achieve a target Eudragit S polymer corresponding to 9.5 mg/cm2 (sample 1). After conducting the sampling of approximately 15 g of Eudragit S coated pellets, the remaining pellets were further coated to a target of 11.5 mg/cm2 (sample 2), followed by a short in-process pre-drying step (40° C. inlet air temperature) for 30 minutes (sample 2 only). After pre-drying sample 2, both samples of final coated pellets were dried for 4 hours in a ventilated oven (0.6 bar airflow) at 40° C.

The dried pellets were characterized to determine the remaining humidity.

### Example 2

Preparation of a Multilayered Formulation with a Sustained Release Layer Comprising the Cationic Polymer Eudragit RS

### 2.1 Manufacture of Drug Layered Pellets

The manufacture of drug-layered pellets was carried out similarly as described in Example 1, section 1.1. The detailed description of the process is provided hereby.

A 20 mMol histidine buffer (1000 ml) was prepared by dispensing 800 ml of purified water into a 1000 ml volumetric cylinder and adding 0.746 g L-Histidine and 3.185 g L-Histidine HCl under magnetic stirring until a clear solution. The magnetic stirrer bar was removed prior to adding additional purified water up to 1000 ml with continued stirring. The pH of the final buffer was measured using a calibrated pH meter (final pH was determined as 5.51).

A quantity of histidine buffer (3.45 g) was dispensed into a glass beaker. The required quantity of Pharmacoat 603 (0.50 g) was added under magnetic. The required quantity of Syloid 244 FP (0.05 g) was added to the glass beaker.

Antibody was pre-diluted with purified water to obtain an antibody stock solution at a concentration of 45 mg/ml. Prior to the pre-dilution with water, the thawed antibody solution (already equilibrated to 25° C.) was homogenized by manually rotating the bottle. Thereafter, the required quantity of MB-001 DS (14.8 g) was dispensed into a plastic beaker. The required quantity of purified water (1.2 g) was added to the beaker under magnetic stirring.

Thereafter, the corresponding amount of the HPMC coating suspension was slowly added to the antibody stock solution in the plastic beaker under magnetic stirring to achieve the final coating formulation for drug layering. The drug layer formulation was mixed for at least 30 minutes by magnetic stirring prior to starting the coating process.

A quantity of cellets 1000 (40 g) were dispensed for drug layer coating. Cellets were coated with the drug layer (antibody) formulation.

The drug layered pellets were removed from the product container at the end of coating process and distributed on a stainless-steel tray. The drug layered pellets were tray dried in a ventilated oven set at 40° C. (0.6 bar airflow) for 22 hours.

A quantity of dried drug layered pellets (35 g) was weighed into a 100 ml brown glass bottle for additional coating trials. The bottle was closed and sealed with parafilm. The remaining quantity of pellets were transferred to a small glass bottle for humidity determination. Pellets were weighed before and after layering and drying to enable calculation of the theoretical antibody loading. The theoretical loading of the antibody was calculated based on the amount of total solids layered after each step (accounting for residual moisture) and the known antibody concentration in the spraying dispersion.

### 2.2 Sustained Release Layer

Drug layered pellets were coated with an aqueous sustained release dispersion containing Eudragit RS as a functional polymer (Eudragit RS 30 D available as a 30% dispersion).

The bottle containing Eudragit RS 30D was manually shaken for 2 minutes. The Eudragit RL 30D was dispensed by pouring through a 0.5 mm mesh into a tared 50 ml glass beaker on a balance until the required quantity (15.385 g) was achieved. The required quantity of purified water (13.231 g) was added to the beaker under magnetic stirring. The required quantity of triethyl citrate (0.923 g) was added to the beaker. The required quantity of Syloid 244 FP (0.462 g) was added to the beaker. Stirring was continued for an additional 30 minutes.

An anti-tacking suspension was prepared by dispensing the required quantity of purified water (4.34 g) into a 20 ml glass beaker. The required quantity of Syloid 244 FP (0.38 g) was added to the beaker under magnetic stirring (200 rpm). Stirring was continued for at least 30 minutes until use.

Approximately 35 g of low antibody-loaded pellets obtained in 2.1 were coated with the Eudragit RS dispersion to a achieve a 6% polymer weight gain. Pellets were coated in the Glatt Mini/Midi coater fitted with the micro kit using the bottom spray configuration.

After the coating process, the anti-tacking dispersion of Syloid 244FP in purified water was sprayed onto the pellets to reduce stickiness (6.25%, based on the target pellet weight after Eudragit RS coating).

Curing was performed in-process for 30 minutes (at a product temperature of 45.5-48.2° C.). During the curing process, 240 g of purified water per kg (starting batch size) was sprayed to create a moist environment required for curing of the polymer.

Approximately 20 g of Eudragit RS coated pellets were dispensed into a 30 ml glass bottle for further coating trials. The remaining pellets were dispensed into an additional 30 ml glass bottle for characterization (LOD, average pellet mass and surface area).

### 2.3 Eudragit S Enteric Coating

The delayed release layer was applied as described in Example 1, section 1.3.

### Example 3 (Comparative Example)

Preparation of a multilayered formulation with a sustained release layer comprising the non-ionic polymer Aquacoat EDC 30

### 3.1 Manufacture of Drug Layered Pellets

The manufacture of drug-layered pellets was carried out as described in Example 1, section 1.1.

### 3.2 Sustained Release Layer

A 30 g Aquacoat ECD 30 dispersion was prepared as described below.

The bottle containing Aquacoat ECD 30 was shaken for 2 minutes. The Aquacoat ECD 30 was dispensed by pouring through a 0.5 mm mesh into a tared 50 ml glass beaker on a balance until the required quantity (16.667 g) was achieved. The required quantity of purified water (12.333 g) was added to the beaker under magnetic stirring (400 rpm for 10 minutes). The required quantity of triethyl citrate (1.000 g) was added to the beaker. Stirring was continued for an additional 10 minutes. Approximately 35 g of low drug loaded pellets were coated with the Aquacoat ECD 30 dispersion. Pellets were coated in the Glatt Mini/Midi coater fitted with the micro kit.

Ethyl cellulose coated pellets were tray dried in a ventilated oven for 2 hours at 60° C. (with 0.6 bar ventilation).

Approximately 20 g of Ethyl cellulose coated pellets were dispensed into a 30 ml glass bottle for further coating trials. The remaining pellets were dispensed into an additional 30 ml glass bottle for characterization (LOD, average pellet mass and surface area).

### 3.3 Eudragit S Enteric Coating

The delayed release layer was applied as described in Example 1, section 1.3., except for step 3 which was conducted as follows.

The Eudragit S organic solution was transferred into a 250 ml glass bottle. The final coating dispersion was prepared by slowly adding the GMS emulsion to the Eudragit S solution whilst stirring at 400 rpm. The bottle was closed and sealed with parafilm. The solution was left stirring overnight.

The compositions of the formulations of Examples 1 to 3 are summarized in table 4.

TABLE 4
Compositions of Example 1, 2 & 3

| Examples: | 1 | 2 | 3 |
|---|---|---|---|
| Batch number | RAVO17-077-1 | CAGO01-155 | RAVO17-090-1 |
| Sustained release | Eudragit | Eudragit | Ethylcellulose |
| polymer weight gain | RL 5.8% | RS 6.4% | (Aquacoat |
| after sustained | | | ECD) 5.8% |
| release coating (target | | | |
| 6.0%) | | | |
| Eudragit S100 after | 9.69 mg/cm² | 9.49 mg/cm² | 8.84 mg/cm² |
| delayed release | | | |
| coating (target Eud | | | |
| S: 9.5 mg/cm²) | | | |

Inert core

| | | | |
|---|---|---|---|
| Cellets | 57.47% | 57.85% | 57.88% |
| (microcrystalline | | | |
| cellulose) | | | |

Drug layer (depot layer)

| | | | |
|---|---|---|---|
| anti-TNFα antibody | 1.14% | 0.80% | 1.14% |
| drug substance | | | |
| (solid) | | | |
| L-Histidine/ | 0.11% | 0.08% | 0.11% |
| L-Histidine | | | |
| HCL buffer salts | | | |
| (solid) | | | |
| Pharmacoat 603 | 0.83% | 0.59% | 0.84% |
| Syloid 244FP | 0.08% | 0.06% | 0.08% |
| Sucrose | 2.00% | 1.42% | 2.02% |
| Polysorbate 80 | 0.01% | 0.01% | 0.01% |

Sustained release layer

| | | | |
|---|---|---|---|
| Eudragit RL (solid) | 3.74% | n/a n/a | n/a n/a |
| Eudragit RS (solid) | n/a n/a | 4.00% | n/a n/a |
| Ethylcellulose | n/a n/a | n/a n/a | 3.77% |
| (Aquacoat EC solid) | | | |
| Triethyl citrate (TEC) | 0.75% | 0.80% | 0.75% |
| Syloid 244FP | 0.37% | 0.40% | n/a n/a |

Delayed release layer

| | | | |
|---|---|---|---|
| Eudragit S100 | 22.99% | 23.40% | 22.60% |
| Glycerol | 2.30% | 2.34% | 2.26% |
| monostearate (GMS) | | | |
| Polysorbate 80 | 0.92% | 0.94% | 0.90% |
| Triethyl citrate (TEC) | 2.30% | 2.34% | 2.26% |
| Humidity | 4.98% | 4.99% | 5.37% |
| (measured by LoD) | | | |
| TOTAL | 100% | 100% | 100% |

### Example 4. Characterization of Delayed Release Pellets of Examples 1-3

### Dissolution Assay

Dissolution with 75 rpm agitation speed at 37° C.

### Media:

a) 0.1 N HCl;
b) potassium phosphate buffer with sodium chloride, pH 6.0. Salts were mixed so that the ionic strength of the medium was equivalent to the ionic strength of biorelevant Hanks buffer (H. M. Fadda, and A. W. Basit, J. Drug Del. Sci. Tech. 2005; 15(4): 273-280).

### Procedure:

Into each vessel, one dosage unit was placed and agitated for 2 h in 0.1 N HCl, followed by 2 h dissolution in medium b). After that, the pH of the dissolution medium was set to pH 6.8. To obtain profiles, samples were analyzed by HPLC and total protein was quantified using the Bradford method as detailed below.

### Antibody Release Using Bradford Determination

Dissolution of anti-TNFα antibody layered pellets, and sustained release coated pellets was performed by rotational mixing in phosphate buffer pH 6.8. Briefly, pellets were weighed in 5 mL cryotubes to which 4 mL phosphate buffer pH 6.8 was added. The tubes were then rotated at 15 rpm in a bench-top rotating device. The amount of coated pellets used per tube was calculated in order to yield a theoretical 0.8 mg/mL antibody drug substance (DS) concentration in the buffer at 100% release, considering the theoretical antibody loading. At defined time points (15 min; 30 min; 1 h; 2 h; 4 h; 6 h and 24 h), 100 μL supernatant were sampled. The samples were then centrifuged for 5 min at 3000 rpm and the supernatant was used for further analysis. The volume of sample taken was not replaced with fresh buffer during the course of the experiments.

Total protein quantification was done by colorimetry following the Bradford method with a Coomassie Plus assay (Thermo Scientific). Quantification was done using a freshly prepared standard calibration curve in 10 mM phosphate buffer pH 6.8. The calibration curve was built with anti-TNFα antibody standards between 0.03 and 1.0 mg/mL.

### Scanning Electron Microscopy

Approximately 25 to 30 coated pellets were stuck on to Scotch tape and fixed on to an A4 size piece of cardboard. The pellets were cut in half using a retractable knife blade. One half of each successfully cut pellet was stuck onto a 25 mm sample stub, cross section facing upwards along with 3 intact pellets for surface determination. Photographs were taken of the cross section with all coating layers. Measurements were taken of each coating layer using the measurement tool of the SEM software. The procedure was repeated for at least 20 cut pellets.

### Results:

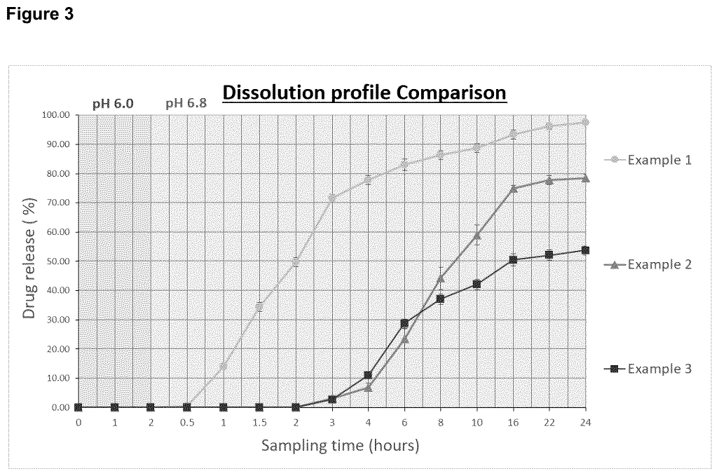

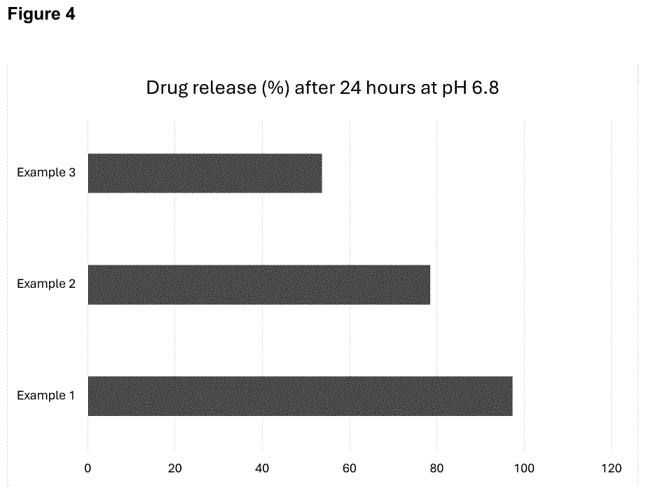

A graphical summary of dissolution results is presented in FIGS. 3 and 4.

In terms of the combinatorial effect between the polymer of the outer layer (e.g., Eudragit S) and the sustained release polymers dictating the overall drug release pattern in dissolution, the hypothesis that the increased amount of quaternary ammonium groups in Eudragit RL might result in a higher level of ionic exchange of Cl— with the COO— groups from the Eudragit S polymer resulting thereby in a faster release profile appears to be correct. This assumption is based upon the drug release patterns of Example 1 (Eudragit S on Eudragit RL) and Example 2 (Eudragit S on Eudragit RS) respectively. Both prototypes did not release during the two hours exposure to pH 6.0 suggesting a successful protection of the antibody from a too early proximal drug release. However, the lag time to start drug release at pH 6.8 was accelerated for the prototype sub-coated with Eudragit RL (Example 1, lag time about 45 min) in comparison to ca. 4 hours lag time to start drug release observed with the prototype sub-coated with Eudragit RS (Example 2).

Interestingly, the rate of drug release (slope of the curve until 70% release) was similar for both combinations, “Eudragit S on RL” (Example 1) and “Eudragit S on RS” (Example 2), but the maximal amount of antibody released after 24 hours was higher for the “Eudragit S on RL” combination (˜100% released) in comparison to 80% obtained with the “Eudragit S on RS” formulation.

In terms of the combinatorial effect explored with drug layered pellets sub-coated with Ethyl cellulose as neutral sustained release polymer and outer coated with Eudragit S (Example 3), in contrast to the Eudragit RL & Eudragit RS polymers, Ethyl cellulose does not contain any functional quaternary ammonium chloride group that could lead to a further activation of the sustained release matrix via ionic exchanges. In that context, the hypothesis that a slower dissolution profile might be achieved in the case of the combination of Ethyl cellulose (EC) and Eudragit S seems to be correct. The lag time to start drug release was similar for the Eudragit S-on-EC combination (Example 3) and the Eudragit S-on-RS formulation (lag time of ca. 4 hours). However, the amount of drug released was not increased by the Eudragit S dissolved in the media in the case of the Ethyl cellulose sub-coated prototype. This resulted in a slower dissolution rate and lower level of antibody released after 24 hours for the Eudragit S-on-EC comparative example (Example 3) in comparison to the Eudragit S-on-RS (Example 2) and Eudragit S-on-RL (Example 1) respectively.

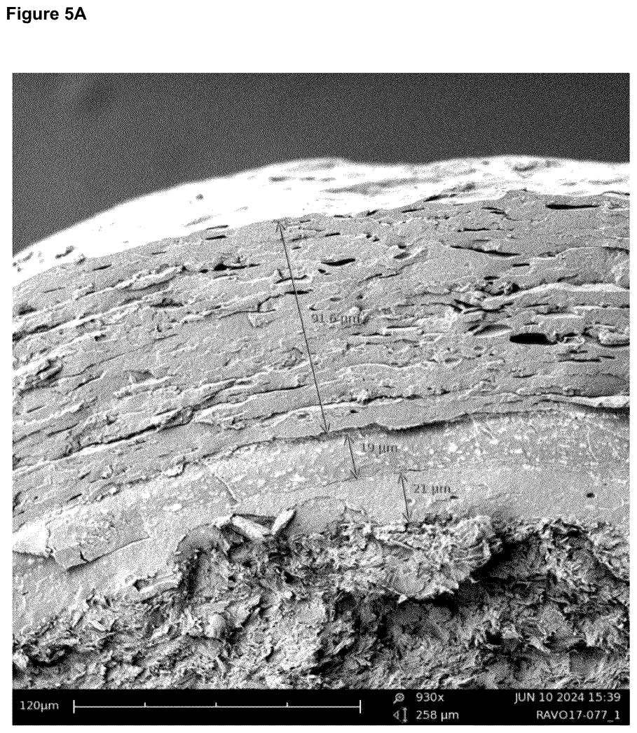

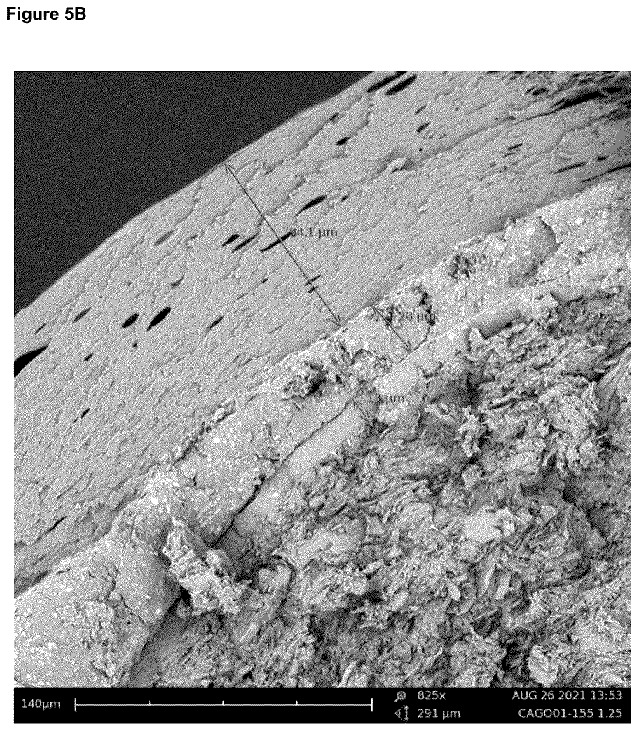

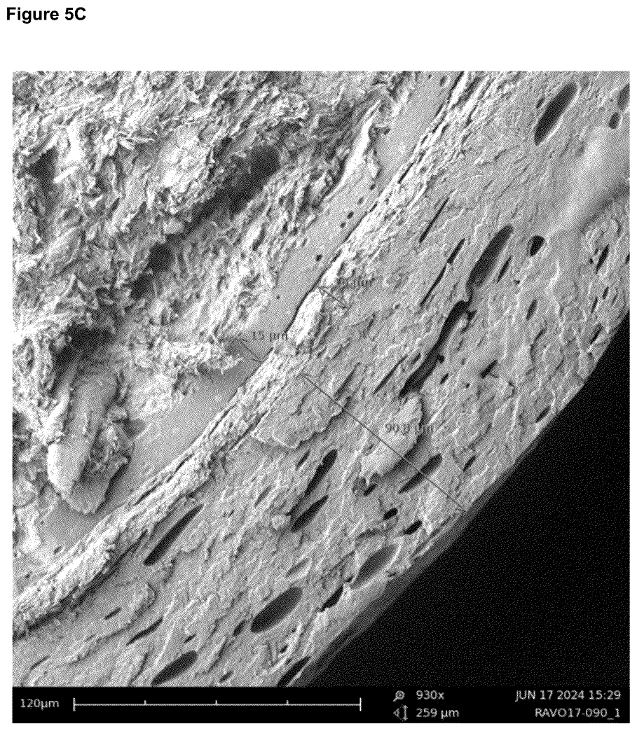

SEM cross-sections are shown in FIG. 5A-5C. In addition, thickness of each coating was determined by scanning electron microscopy as described supra. Data validated the suitability of the coating process for each prototype to achieve the objective of uniform and similar coating thickness.

### Example 5. Formulation Examples

Three delayed-release formulations in accordance with the present invention were prepared, designated Examples 5.1, 5.2, and 5.3, respectively. These formulations had a higher drug load than Examples 1 and 2. The compositions were as follows. Table 4: Composition of Examples 5.1, 5.2 and 5.3 (values in percentage are weight percentages of constituents in individual layers, relative to the total weight of the final composition of Example 5.1, 5.2 and 5.3, resp.)

Examples 5.1, 5.2 and 5.3

| Eudragit RS weight gain after sustained release | | | (target Eud |
|---|---|---|---|
| coating (target Eud RS: 6.0%) | | | RS: 6.0%) |
| Eudragit S100 after delayed release coating | | | (target Eud |
| (target Eud S: 9.5 mg/cm²) | | | S: 11.50 |
| | | | mg/cm²) |
| Composition | Example 5.1 | Example 5.2 | Example 5.3 |

Inert core

| | | | |
|---|---|---|---|
| Cellets (microcrystalline | 27.16% | 25.22% | 25.36% |
| cellulose) | | | |

Drug layer (depot layer)

| | | | |
|---|---|---|---|
| anti-TNFα antibody drug | 9.48% | 9.26% | 19.01% |
| substance (solid) | | | |
| L-Histidine/L-Histidine | 0.94% | 0.91% | 0.88% |
| HCL buffer | | | |
| salts (solid) | | | |
| Pharmacoat 603 | 6.90% | 6.74% | 6.58% |
| Syloid 244FP | 0.69% | 0.67% | 0.66% |
| Sucrose | 16.87% | 16.48% | 15.87% |
| Polysorbate 80 | 0.08% | 0.08% | 0.08% |

Sustained release layer

| | | | |
|---|---|---|---|
| Eudragit RS (solid) | 3.94% | 3.80% | 3.68% |
| Triethyl citrate (TEC) | 0.79% | 0.76% | 0.74% |
| Syloid 244FP | 0.39% | 0.38% | 0.37% |

Delayed release layer

| | | | |
|---|---|---|---|
| Eudragit S100 | 22.93% | 24.80% | 25.71% |
| Glycerol monostearate (GMS) | 2.29% | 2.48% | 2.57% |
| Polysorbate 80 | 0.92% | 0.99% | 1.03% |
| Triethyl citrate (TEC) | 2.29% | 2.48% | 2.57% |
| Humidity (measured by LoD) | 4.33% | 4.95% | 4.90% |
| TOTAL | 100% | 100% | 100% |

### Example 6. Stability Tests

Formulations in accordance with Example 5.3 were stored at either 2-8° C. or at 25° C./60% RH for 12 months. At various time points the potency of the antibody in the formulation was tested by determining the relative potency (against nominal antibody content) of the anti-TNFalpha antibody, using a cell-based assay adapted for the antibody.

### Exemplary Potency Assay:

A549 cells are seeded prior to the assay. A dilution series of the analyte is incubated with a constant dose of TNFalpha. After allowing the formation of analyte-TNFalpha complexes, the A549 cells are subsequently stimulated with the mixture and the stimulation abrogated by fixing the cells with Paraformaldehyde. The phosphorylated NF-κB S536 is labeled by consecutive staining of the cells with a phosphor-specific NF-κB S536 antibody and a HRP-labeled detection antibody. The amount of HRP is detected by the conversion of a chemiluminescent substrate by HRP and the generation of a luminescent signal which can be captured on specialized instruments.

### Results:

| time point | relative potency (%) | |
|---|---|---|
| (months after | storage at | storage at 25° C./ |
| start of incubation) | 2-8° C. | 60% RH |
| 0 (initial) | 97 | |
| 1 | 103 | 102 |
| 3 | 100 | 103 |
| 6 | 103 | 98 |
| 9 | 104 | 94 |
| 12 | 103 | 95 |

As can be seen, the antibody retains its activity over a period of at least 12 months at both 2-8° C. and 25° C./60% RH. The stability at 25° C. is very remarkable and surprising for an antibody drug.

1. A solid oral dosage form comprising:
i) an inert core unit (1);
ii) a depot layer (2) covering the inert core unit (1) and comprising an antibody or a functional fragment thereof, as an active agent; and optionally a stabilizer, a buffer and/or a polymeric binder;
iii) a sustained release layer (3), covering the depot layer (2) and comprising at least one cationic polymer; and optionally a plasticizer and/or an anti-tacking agent; and
iv) a delayed release layer (4) covering the sustained release layer (3) and comprising at least one anionic polymer and optionally a plasticizer.

2. A solid oral dosage form comprising:
i) an inert core unit (1);
ii) a depot layer (2) covering the inert core unit (1) and comprising an antibody or a functional fragment thereof as an active agent; a stabilizer; a buffer; and a polymeric binder;
iii) a sustained release layer (3), covering the depot layer (2) and comprising at least one cationic polymer; a plasticizer; and an anti-tacking agent; and
iv) a delayed release layer (4) covering the sustained release layer (3) and comprising at least one anionic polymer and a plasticizer.

3. The solid dosage form of claim 1, wherein the at least one anionic polymer is selected from the group consisting of polymers comprising carboxylic acid groups; poly(methacrylic acid, methyl methacrylate) 1:1; poly(methacrylic acid, ethyl acrylate) 1:1; poly(methacrylic acid, methyl methacrylate) 1:2; poly(methyl acrylate, methyl methacrylate, methacrylic acid) 7:3:1; carboxymethyl cellulose; and combinations thereof.

4. The solid dosage form of claim 1, wherein the at least one cationic polymer is selected from the group consisting of chitosan; cellulose; ammonio methacrylate copolymers; poly(ethyl acrylate, methyl methacrylate, trimethylammonioethyl methacrylate chloride) 1:2:0.1; poly(2-N,N-dimethylaminoethylmethacrylate); poly-L-lysine; polyethylenimine; Poly(amidoamine).

5. A solid oral dosage form comprising:
i) an inert core unit (1);
ii) a depot layer (2) covering the inert core unit (1) and comprising an antibody or a functional fragment thereof, as an active agent; a stabilizer; a buffer; and a polymeric binder;
iii) a sustained release layer (3), covering the depot layer (2) and comprising an ammonio methacrylate copolymer; a plasticizer; and an anti-tacking agent; and
iv) a delayed release layer (4) covering the sustained release layer (3) and comprising poly(methacrylic acid, methyl methacrylate) 1:2, poly(methacrylic acid, methyl methacrylate) 1:1, or a combination thereof; an anti-tacking agent; and a plasticizer.

6. The solid dosage form of claim 1, wherein the sustained release layer does not comprise an anionic polymer; and/or wherein the delayed release layer does not comprise a cationic polymer.

7. (canceled)

8. The solid oral dosage form of claim 1, wherein the stabilizer in the depot layer (2) is selected from sucrose, maltose, lactose, trehalose, glycerol, maltitol, isomalt, mannitol, sorbitol, xylitol, and combinations thereof, preferably sucrose.

9. The solid oral dosage form of claim 1, wherein the buffer in the depot layer (2) is selected from the group consisting of L-histidine buffer, citrate buffer, hydroxymethylaminomethane (TRIS) buffer, succinate buffer, phosphate buffer, acetate buffer, and combinations thereof.

10. The solid oral dosage form of claim 1, wherein in the depot layer (2) the stabilizer is sucrose; and/or the buffer is L-histidine or a salt thereof, and/or the polymeric binder in the depot layer (2) is hypromellose (HPMC).

11. The solid oral dosage form of claim 1, wherein the depot layer (2) comprises mesoporous silica as an anti-tacking agent and/or polysorbate 80 as a surfactant.

12. (canceled)

13. The solid oral dosage form of claim 1, wherein sustained release layer (3) comprises poly(ethyl acrylate, methyl methacrylate, trimethylammonioethyl methacrylate chloride) 1:2:0.1 and no other sustained release polymer.

14. The solid oral dosage form of claim 1, wherein in the sustained release layer (3) the plasticizer is triethyl citrate (TEC); and/or the anti-tacking agent is mesoporous silica.

15. The solid oral dosage form of claim 1, wherein in the delayed release layer (4) the plasticizer is triethyl citrate (TEC); and/or the anti-tacking agent is glycerol monostearate (GMS).

16. The solid oral dosage form of claim 1, wherein the delayed release layer (4) comprises, poly(methacrylic acid, methyl methacrylate) 1:2, and no other delayed release polymers.

17. The solid oral dosage form of claim 1, wherein the delayed release layer (4) comprises a surfactant, optionally wherein the surfactant in the delayed release layer (4) is polysorbate 80.

18. (canceled)

19. The solid oral dosage form of claim 1, wherein the inert core unit (1) is a pellet with a particle size distribution such that at least 85% of the pellets have a particle size of 700-1400 μm.

20. (canceled)

21. The solid oral dosage form of claim 1, wherein the depot layer (2) comprises 0.1-20 wt.-% antibody or functional fragment thereof, 0.1-25 wt.-% binder, 0.5-35 wt.-% sucrose, 0.01-2 wt.-% L-histidine and optionally 0.01-3 wt.-% other buffers, 0.01-5 wt.-% anti-tacking agent, and/or 0.001-1 wt.-% polysorbate 80, relative to the total weight of the solid oral dosage form.

22. The solid oral dosage form of claim 1, wherein the sustained release layer (3) comprises 3-6 wt.-% ammonio methacrylate copolymer, 0.2-2 wt.-% plasticizer and/or 0.1-3 wt.-% anti-tacking agent, relative to the total weight of the solid oral dosage form.

23. The solid oral dosage form of claim 1, wherein the delayed release layer (4) comprises 15-35 wt.-% poly(methacrylic acid, methyl methacrylate) 1:2, 1-4 wt.-% plasticizer, 0.2-2 wt.-% surfactant, and/or 1-5 wt.-% glycerol monostearate (GMS), relative to the total weight of the solid oral dosage form.

24-30. (canceled)

31. The solid oral dosage form of claim 1, wherein the solid oral dosage form comprises, or consists of:
a. in the inert core unit (1), 25.21-25.51 wt.-% microcrystalline cellulose;
b. in the depot layer (2), 8.91-9.11 wt.-% antibody or functional fragment thereof, 6.48-6.68 wt.-% hypromellose (HPMC), 15.72-16.02 wt.-% sucrose, 0.83-0.93 wt.-% L-histidine, 0.61-0.71 wt.-% mesoporous silica, and 0.07-0.09 wt.-% polysorbate 80;
c. in the sustained release layer (3), 3.58-3.78 wt.-% poly(ethyl acrylate, methyl methacrylate, trimethylammonioethyl methacrylate chloride) 1:2:0.1, 0.69-0.79 wt. % triethyl citrate (TEC) and 0.32-0.42 wt.-% mesoporous silica;
d. in the delayed release layer (4), 25.56-25.86 wt.-% poly(methacrylic acid, methyl methacrylate) 1:2, 2.52-2.62 wt.-% triethyl citrate (TEC), 0.98-1.08 wt.-% polysorbate 80, and 2.52-2.62 wt.-% glycerol monostearate (GMS); and
e. 4.80 wt.-% to 5.00 wt.-%, water in the inert core unit and/or any of the layers.

32. (canceled)

33. The solid oral dosage form of claim 1, wherein the antibody or functional fragment thereof is an antibody specific to tumor necrosis factor alpha (TNFα) or a functional fragment thereof.

34. The solid oral dosage form of claim 33, wherein the antibody specific to TNFα or functional fragment thereof is an anti-TNFα antibody or functional fragment thereof with a TNFα binding domain comprising (i) a V_{L} domain comprising a CDR1 region having an amino acid sequence as shown in SEQ ID NO:1, a CDR2 region having an amino acid sequence as shown in SEQ ID NO:2, and a CDR3 region having an amino acid sequence as shown in SEQ ID NO:3, and/or (ii) a V_{H} domain comprising a CDR1 region having an amino acid sequence as shown in SEQ ID NO:4, a CDR2 region having an amino acid sequence as shown in SEQ ID NO:5, and a CDR3 region having the amino acid sequence as shown in SEQ ID NO: 6.

35. (canceled)

36. The solid oral dosage form of claim 33, wherein the antibody specific to TNFα or functional fragment thereof is an anti-TNFα antibody comprising an Fc region having, or consisting of, an amino acid sequence in accordance with the amino acid sequence as shown in SEQ ID NO:18, in SEQ ID NO:19, in SEQ ID NO:20, in SEQ ID NO:21, or in SEQ ID NO:22, preferably in SEQ ID NO: 18.

37. (canceled)

38. (canceled)

39. (canceled)

40. (canceled)

41. A method for targeted local treatment of a gastrointestinal disease, comprising administering to a patient in need thereof a pharmaceutically effective amount of the solid oral dosage form of claim 1 or an oral multiparticulate drug delivery system comprising a plurality of said solid oral dosage forms.

42. The method of claim 41, wherein the gastrointestinal disease is immune checkpoint inhibitor (ICPI) induced colitis, ICPI induced enterocolitis, ICPI induced diarrhoea, or an inflammatory bowel disease (IBD).


SEQUENCE LISTING (35 sequences)

SEQ ID NO: 1 - protein, 11 aa, synthetic construct
  REGION 1..11: note = CDR L1 of clone 16-22-H05
QASQSIFSGLA

SEQ ID NO: 2 - protein, 7 aa, synthetic construct
  REGION 1..7: note = CDR L2 of clone 16-22-H05
GASKLAS

SEQ ID NO: 3 - protein, 15 aa, synthetic construct
  REGION 1..15: note = CDR L3 of clone 16-22-H05
QSYYYSSSSSDGSYA

SEQ ID NO: 4 - protein, 10 aa, synthetic construct
  REGION 1..10: note = CDR H1 of clone 16-22-H05
GIDFNNYGIG

SEQ ID NO: 5 - protein, 17 aa, synthetic construct
  REGION 1..17: note = CDR H2 of clone 16-22-H05
YIYPGFAITNFANSVKG

SEQ ID NO: 6 - protein, 13 aa, synthetic construct
  REGION 1..13: note = CDR H3 of clone 16-22-H05
DPVYATSSGYFDL

SEQ ID NO: 7 - protein, 122 aa, synthetic construct
  REGION 1..122: note = VH of humanized IgG of clone 16-22-H05
EVQLVESGGGLVQPGGSLRLSCKASGIDFNNYGIGWVRQAPGKGLEWITYIYPGFAITNFANSVKGRFTISSDNSKNTVYLQMNSLRAEDTAVYYCARDPVYATSSGYFDLWGQGTLVTVSS

SEQ ID NO: 8 - protein, 114 aa, synthetic construct
  REGION 1..114: note = VL of humanized IgG of clone 16-22-H05
DIQMTQSPSSLSASVGDRVTIKCQASQSIFSGLAWYQQKPGKAPKLLIYGASKLASGVPSRFSGSGSGTEFTLTISGLQPADFATYYCQSYYYSSSSSDGSYAFGGGTKLTVLG

SEQ ID NO: 9 - protein, 11 aa, synthetic construct
  REGION 1..11: note = CDR L1 of clone 17-22-B03
QASQSISSYLA

SEQ ID NO: 10 - protein, 7 aa, synthetic construct
  REGION 1..7: note = CDR L2 of clone 17-22-B03
KASTLAS

SEQ ID NO: 11 - protein, 13 aa, synthetic construct
  REGION 1..13: note = CDR L3 of clone 17-22-B03
QCTYYEPSYVFRA

SEQ ID NO: 12 - protein, 11 aa, synthetic construct
  REGION 1..11: note = CDR H1 of clone 17-22-B03
GIDFSAGYDMC

SEQ ID NO: 13 - protein, 17 aa, synthetic construct
  REGION 1..17: note = CDR H2 of clone 17-22-B03
CIDSRREESDYASWAKG

SEQ ID NO: 14 - protein, 14 aa, synthetic construct
  REGION 1..14: note = CDR H3 of clone 17-22-B03
GGYGGDGVDGAFDP

SEQ ID NO: 15 - protein, 124 aa, synthetic construct
  REGION 1..124: note = VH of humanized IgG of clone 17-22-B03
EVQLVESGGGLVQPGGSLRLSCKASGIDFSAGYDMCWVRQAPGKGLEWIACIDSRREESDYASWAKGRFTISKDSSKNTVYLQMNSLRAEDTAVYYCARGGYGGDGVDGAFDPWGQGTLVTVSS

SEQ ID NO: 16 - protein, 112 aa, synthetic construct
  REGION 1..112: note = VL of humanized IgG of clone 17-22-B03-(sc08)
DIQMTQSPSSLSASVGDRVTITCQASQSISSYLAWYQQKPGKAPKLLIYKASTLASGVPSRFSGSGSGTDFTLTISSLQPEDFATYYCQCTYYEPSYVFRAFGGGTKLTVLG

SEQ ID NO: 17 - protein, 112 aa, synthetic construct
  REGION 1..112: note = VL of humanized IgG of clone 17-22-B03-(sc02)
DIQMTQSPSSLSASVGDRVTIKCQASQSISSYLAWYQQKPGKAPKLLIYKASTLASGVPSRFSGSGSGTEFTLTISDLEPADFATYYCQCTYYEPSYVFRAFGGGTKLTVLG

SEQ ID NO: 18 - protein, 231 aa, synthetic construct
  REGION 1..231: note = Fc region of Ab-REW (including hinge region)
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVEHEALHWHYTQKSLSLSPG

SEQ ID NO: 19 - protein, 230 aa, synthetic construct
  REGION 1..230: note = Fc region of Ab-YTE-DLE-PVAdeltaG (including hinge region)
EPKSCDKTHTCPPCPAPPVAGPDVFLFPPKPKDTLYITREPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 230 aa, synthetic construct
  REGION 1..230: note = Fc region of Ab-A-DLE-PVAdeltaG(including hinge region)
EPKSCDKTHTCPPCPAPPVAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 21 - protein, 230 aa, synthetic construct
  REGION 1..230: note = Fc region of Ab-A-AEA-PVAdeltaG(including hinge region)
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPEAKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 22 - protein, 231 aa, synthetic construct
  REGION 1..231: note = Fc region of Ab-AA (including hinge region)
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVAWESNGQPENNYKTTPPVLDSDGSFFLYSKLTVDKSRWQQGNVFSCSVMHEALHAHYTQKSLSLSPG

SEQ ID NO: 23 - protein, 221 aa, synthetic construct
  REGION 1..221: note = Light chain of Ab-wt, the parent antibody (clone 16-22-H05)
DIQMTQSPSSLSASVGDRVTIKCQASQSIFSGLAWYQQKPGKAPKLLIYGASKLASGVPSRFSGSGSGTEFTLTISGLQPADFATYYCQSYYYSSSSSDGSYAFGGGTKLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 219 aa, synthetic construct
  REGION 1..219: note = Light chain of humanized IgG of clone 17-22-B03-(sc08)
DIQMTQSPSSLSASVGDRVTITCQASQSISSYLAWYQQKPGKAPKLLIYKASTLASGVPSRFSGSGSGTDFTLTISSLQPEDFATYYCQCTYYEPSYVFRAFGGGTKLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 219 aa, synthetic construct
  REGION 1..219: note = Light chain of humanized IgG of clone 17-22-B03-(sc02)
DIQMTQSPSSLSASVGDRVTIKCQASQSISSYLAWYQQKPGKAPKLLIYKASTLASGVPSRFSGSGSGTEFTLTISDLEPADFATYYCQCTYYEPSYVFRAFGGGTKLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 451 aa, synthetic construct
  REGION 1..451: note = Heavy chain of Ab-REW (based on clone 16-22-H05)
EVQLVESGGGLVQPGGSLRLSCKASGIDFNNYGIGWVRQAPGKGLEWITYIYPGFAITNFANSVKGRFTISSDNSKNTVYLQMNSLRAEDTAVYYCARDPVYATSSGYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVEHEALHWHYTQKSLSLSPG

SEQ ID NO: 27 - protein, 453 aa, synthetic construct
  REGION 1..453: note = Heavy chain of Ab-REW (based on clone 17-22-B03)
EVQLVESGGGLVQPGGSLRLSCKASGIDFSAGYDMCWVRQAPGKGLEWIACIDSRREESDYASWAKGRFTISKDSSKNTVYLQMNSLRAEDTAVYYCARGGYGGDGVDGAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHRDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVEHEALHWHYTQKSLSLSPG

SEQ ID NO: 28 - protein, 450 aa, synthetic construct
  REGION 1..450: note = Heavy chain of Ab-YTE-DLE-PVAdeltaG (based on clone 16-22-H05)
EVQLVESGGGLVQPGGSLRLSCKASGIDFNNYGIGWVRQAPGKGLEWITYIYPGFAITNFANSVKGRFTISSDNSKNTVYLQMNSLRAEDTAVYYCARDPVYATSSGYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPDVFLFPPKPKDTLYITREPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 450 aa, synthetic construct
  REGION 1..450: note = Heavy chain of Ab-A-DLE-PVAdeltaG (based on clone 16-22-H05)
EVQLVESGGGLVQPGGSLRLSCKASGIDFNNYGIGWVRQAPGKGLEWITYIYPGFAITNFANSVKGRFTISSDNSKNTVYLQMNSLRAEDTAVYYCARDPVYATSSGYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 30 - protein, 450 aa, synthetic construct
  REGION 1..450: note = Heavy chain of Ab-A-AEA-PVAdeltaG (based on clone 16-22-H05)
EVQLVESGGGLVQPGGSLRLSCKASGIDFNNYGIGWVRQAPGKGLEWITYIYPGFAITNFANSVKGRFTISSDNSKNTVYLQMNSLRAEDTAVYYCARDPVYATSSGYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPEAKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 31 - protein, 452 aa, synthetic construct
  REGION 1..452: note = Heavy chain of Ab-YTE-DLE-PVAdeltaG (based on clone 17-22-B03)
EVQLVESGGGLVQPGGSLRLSCKASGIDFSAGYDMCWVRQAPGKGLEWIACIDSRREESDYASWAKGRFTISKDSSKNTVYLQMNSLRAEDTAVYYCARGGYGGDGVDGAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPDVFLFPPKPKDTLYITREPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 452 aa, synthetic construct
  REGION 1..452: note = Heavy chain of Ab-A-DLE-PVAdeltaG (based on clone 17-22-B03)
EVQLVESGGGLVQPGGSLRLSCKASGIDFSAGYDMCWVRQAPGKGLEWIACIDSRREESDYASWAKGRFTISKDSSKNTVYLQMNSLRAEDTAVYYCARGGYGGDGVDGAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 33 - protein, 452 aa, synthetic construct
  REGION 1..452: note = Heavy chain of Ab-A-AEA-PVAdeltaG (based on clone 17-22-B03)
EVQLVESGGGLVQPGGSLRLSCKASGIDFSAGYDMCWVRQAPGKGLEWIACIDSRREESDYASWAKGRFTISKDSSKNTVYLQMNSLRAEDTAVYYCARGGYGGDGVDGAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPEAKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 34 - protein, 451 aa, synthetic construct
  REGION 1..451: note = Heavy chain of Ab-AA (based on clone 16-22-H05)
EVQLVESGGGLVQPGGSLRLSCKASGIDFNNYGIGWVRQAPGKGLEWITYIYPGFAITNFANSVKGRFTISSDNSKNTVYLQMNSLRAEDTAVYYCARDPVYATSSGYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVAWESNGQPENNYKTTPPVLDSDGSFFLYSKLTVDKSRWQQGNVFSCSVMHEALHAHYTQKSLSLSPG

SEQ ID NO: 35 - protein, 453 aa, synthetic construct
  REGION 1..453: note = Heavy chain of Ab-AA (based on clone 17-22-B03)
EVQLVESGGGLVQPGGSLRLSCKASGIDFSAGYDMCWVRQAPGKGLEWIACIDSRREESDYASWAKGRFTISKDSSKNTVYLQMNSLRAEDTAVYYCARGGYGGDGVDGAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVAWESNGQPENNYKTTPPVLDSDGSFFLYSKLTVDKSRWQQGNVFSCSVMHEALHAHYTQKSLSLSPG